SEQUENCE LISTING (53 sequences)

SEQ ID NO: 1 - DNA, 25 bp, Artificial Sequence
  Synthetic oligonucleotide
tgcctggaatattggtgaacagaaa

SEQ ID NO: 2 - DNA, 25 bp, Artificial Sequence
  Synthetic oligonucleotide
ggtactggtcaggcaataacagtta

SEQ ID NO: 3 - DNA, 23 bp, Artificial Sequence
  Synthetic oligonucleotide
tcactcaacatggcaaggaagac

SEQ ID NO: 4 - DNA, 19 bp, Artificial Sequence
  Synthetic oligonucleotide
cgcattggcatggaagtca

SEQ ID NO: 5 - DNA, 29 bp, Artificial Sequence
  Synthetic oligonucleotide
tgttaaaaacagtacaattctgtgatgcc

SEQ ID NO: 6 - DNA, 23 bp, Artificial Sequence
  Synthetic oligonucleotide
tgtatacagcttgctctcatgcc

SEQ ID NO: 7 - DNA, 22 bp, Artificial Sequence
  Synthetic oligonucleotide
agggccaattctgctgtcaaat

SEQ ID NO: 8 - DNA, 25 bp, Artificial Sequence
  Synthetic oligonucleotide
gggagccttgaatacaccaaaagat

SEQ ID NO: 9 - DNA, 19 bp, Artificial Sequence
  Synthetic oligonucleotide
agatttggacctgcgagcg

SEQ ID NO: 10 - DNA, 18 bp, Artificial Sequence
  Synthetic oligonucleotide
tgtcgccccaagcaactt

SEQ ID NO: 11 - DNA, 23 bp, Artificial Sequence
  Synthetic oligonucleotide
agtgcgggagaaaattgatcgta

SEQ ID NO: 12 - DNA, 20 bp, Artificial Sequence
  Synthetic oligonucleotide
atctatgtggcaacggcagt

SEQ ID NO: 13 - DNA, 25 bp, Artificial Sequence
  Synthetic oligonucleotide
cggtagtagccaatttggtcatctg

SEQ ID NO: 14 - DNA, 22 bp, Artificial Sequence
  Synthetic oligonucleotide
tccaatttgatggcacctgtgt

SEQ ID NO: 15 - DNA, 30 bp, Artificial Sequence
  Synthetic oligonucleotide
aaatcataccagttaccattgagatcttga

SEQ ID NO: 16 - DNA, 30 bp, Artificial Sequence
  Synthetic oligonucleotide
cgtgcaggtataattctactacatttatct

SEQ ID NO: 17 - DNA, 22 bp, Artificial Sequence
  Synthetic oligonucleotide
tcatcagtgcaagcagtttgtg

SEQ ID NO: 18 - DNA, 23 bp, Artificial Sequence
  Synthetic oligonucleotide
ccttgaggaagttgtagcacgat

SEQ ID NO: 19 - DNA, 20 bp, Artificial Sequence
  Synthetic oligonucleotide
gagcggctgtctccacaagt

SEQ ID NO: 20 - DNA, 20 bp, Artificial Sequence
  Synthetic oligonucleotide
gcctgtgaacgcgagttaga

SEQ ID NO: 21 - DNA, 23 bp, Artificial Sequence
  Synthetic oligonucleotide
caacacgagcagcctctgatgca

SEQ ID NO: 22 - DNA, 25 bp, Artificial Sequence
  Synthetic oligonucleotide
acacgatgcaccaccaaaggattct

SEQ ID NO: 23 - DNA, 30 bp, Artificial Sequence
  Synthetic oligonucleotide
acaaggcgttccaattaacaccaatagcag

SEQ ID NO: 24 - DNA, 24 bp, Artificial Sequence
  Synthetic oligonucleotide
caccttcgggaacgtggttgacct

SEQ ID NO: 25 - DNA, 29 bp, Artificial Sequence
  Synthetic oligonucleotide
tcagtacaccaacaataccagcatttcgc

SEQ ID NO: 26 - DNA, 29 bp, Artificial Sequence
  Synthetic oligonucleotide
aatgccttctcacatagtgcatcaacagc

SEQ ID NO: 27 - DNA, 25 bp, Artificial Sequence
  Synthetic oligonucleotide
tagtaccggcagcacaagacatctg

SEQ ID NO: 28 - DNA, 24 bp, Artificial Sequence
  Synthetic oligonucleotide
ttggcacccgcaatcctgctaaca

SEQ ID NO: 29 - DNA, 23 bp, Artificial Sequence
  Synthetic oligonucleotide
ttctgacctgaaggctctgcgcg

SEQ ID NO: 30 - DNA, 24 bp, Artificial Sequence
  Synthetic oligonucleotide
atccattcagcgaccccgttagcg

SEQ ID NO: 31 - DNA, 97 bp, Artificial Sequence
  Synthetic oligonucleotide
tgcctggaatattggtgaacagaaatcaatactgagtcctctttatgcatttgcatcagaggctgctcgtgttgtacgatcaattttctcccgcact

SEQ ID NO: 32 - DNA, 99 bp, Artificial Sequence
  Synthetic oligonucleotide
ggtactggtcaggcaataacagttacaccggaagccaatatggatcaagaatcctttggtggtgcatcgtgttgtctgtactgccgttgccacatagat

SEQ ID NO: 33 - DNA, 97 bp, Artificial Sequence
  Synthetic oligonucleotide
tcactcaacatggcaaggaagaccttaaattccctcgaggacaaggcgttccaattaacaccaatagcagtccagatgaccaaattggctactaccg

SEQ ID NO: 34 - DNA, 65 bp, Artificial Sequence
  Synthetic oligonucleotide
cgcattggcatggaagtcacaccttcgggaacgtggttgacctacacaggtgccatcaaattgga

SEQ ID NO: 35 - DNA, 100 bp, Artificial Sequence
  Synthetic oligonucleotide
tgttaaaaacagtacaattctgtgatgccatgcgaaatgctggtattgttggtgtactgacattagataatcaagatctcaatggtaactggtatgattt

SEQ ID NO: 36 - DNA, 97 bp, Artificial Sequence
  Synthetic oligonucleotide
tgtatacagcttgctctcatgccgctgttgatgcactatgtgagaaggcattaaaatatttgcctatagataaatgtagtagaattatacctgcacg

SEQ ID NO: 37 - DNA, 104 bp, Artificial Sequence
  Synthetic oligonucleotide
agggccaattctgctgtcaaattacagaataatgagcttagtcctgttgcactacgacagatgtcttgtgctgccggtactacacaaactgcttgcactgatga

SEQ ID NO: 38 - DNA, 84 bp, Artificial Sequence
  Synthetic oligonucleotide
gggagccttgaatacaccaaaagatcacattggcacccgcaatcctgctaacaatgctgcaatcgtgctacaacttcctcaagg

SEQ ID NO: 39-40 - skipped (2 entries)

SEQ ID NO: 41 - DNA, 1466 bp, Severe acute respiratory syndrome coronavirus 2
gcggaaggccgtcaaggccacgtgtcttgtccgcggtaccctaagctaatacgactcactatagggagattacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcaatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccatgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgcgcttcagcgttcttcggaatgtcgcgcattggcatggaagtcacaccttcgggaacgtggttgacctacacaggtgccatcaaattggatgacaaagatccaaatttcaaagatctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaaatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacggcatcatatgggttgcaactgagggagccttgaatacaccaaaagatcacattggcacccgcaatcctgctaacaatgctgcaatcgtgctacaacttcctcaaggaacaacattgccaaaaggcttctacagatctaagcgtggaagtggctatatagagcagatatattatatgacgtaccagaggatcacctactaaaagacttttcgagaatctacacctactagcaagggtatagatacatgcggagggaagctcatcagtggggccacgagctgagtgcgtcctgtcactccactcccatgtcccttgggaaggtctgagactagggacgcgtaactgcgagctcggagcacaagactggcctcatgggccttccgctc

SEQ ID NO: 42 - DNA, 357 bp, Homo sapiens
gagcgcgacgtaatacgactcactatagggcgaattggcggaaggccgtcaaggccgcatctaagctaatacgactcactatagggagaagatctaagcgtggaagtggctatatagagcagatatattatatgacgtaccagaggatcacctactaaaagacttttcgagaatctacacctactagcaagggtatggcggtgtttgcagatttggacctgcgagcgggttctgacctgaaggctctgcgcggacttgtggagacagccgctcaccgtgagttgcacgcgtaactgcctgggcctcatgggccttccgctcactgcccgctttccagtcgggaaacctgtcgtgcca

SEQ ID NO: 43 - RNA, 1466 nt, Severe acute respiratory syndrome coronavirus 2
gcggaaggccgucaaggccacgugucuuguccgcgguacccuaagcuaauacgacucacuauagggagauuacaaaaggaaaagcuaaaaaaggugccuggaauauuggugaacagaaaucaauacugaguccucuuuaugcauuugcaucagaggcugcucguguuguacgaucaauuuucucccgcacucuugaaacugcucaaaauucugugcaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccauguuaagauguuguguacacacacugguacuggucaggcaauaacaguuacaccggaagccaauauggaucaagaauccuuugguggugcaucguguugucuguacugccguugccacauagaucauccaaauccuaaaggauuuugugcgcuucagcguucuucggaaugucgcgcauuggcauggaagucacaccuucgggaacgugguugaccuacacaggugccaucaaauuggaugacaaagauccaaauuucaaagaucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaaauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacggcaucauauggguugcaacugagggagccuugaauacaccaaaagaucacauuggcacccgcaauccugcuaacaaugcugcaaucgugcuacaacuuccucaaggaacaacauugccaaaaggcuucuacagaucuaagcguggaaguggcuauauagagcagauauauuauaugacguaccagaggaucaccuacuaaaagacuuuucgagaaucuacaccuacuagcaaggguauagauacaugcggagggaagcucaucaguggggccacgagcugagugcguccugucacuccacucccaugucccuugggaaggucugagacuagggacgcguaacugcgagcucggagcacaagacuggccucaugggccuuccgcuc

SEQ ID NO: 44 - RNA, 357 nt, Homo sapiens
gagcgcgacguaauacgacucacuauagggcgaauuggcggaaggccgucaaggccgcaucuaagcuaauacgacucacuauagggagaagaucuaagcguggaaguggcuauauagagcagauauauuauaugacguaccagaggaucaccuacuaaaagacuuuucgagaaucuacaccuacuagcaaggguauggcgguguuugcagauuuggaccugcgagcggguucugaccugaaggcucugcgcggacuuguggagacagccgcucaccgugaguugcacgcguaacugccugggccucaugggccuuccgcucacugcccgcuuuccagucgggaaaccugucgugcca

SEQ ID NO: 45 - DNA, 1466 bp, Severe acute respiratory syndrome coronavirus 2
gagcggaaggcccatgaggccagtcttgtgctccgagctcgcagttacgcgtccctagtctcagaccttcccaagggacatgggagtggagtgacaggacgcactcagctcgtggccccactgatgagcttccctccgcatgtatctatacccttgctagtaggtgtagattctcgaaaagtcttttagtaggtgatcctctggtacgtcatataatatatctgctctatatagccacttccacgcttagatctgtagaagccttttggcaatgttgttccttgaggaagttgtagcacgattgcagcattgttagcaggattgcgggtgccaatgtgatcttttggtgtattcaaggctccctcagttgcaacccatatgatgccgttgtgttgtagtaagctaacgcattgtcatcagtgcaagcagtttgtgtagtaccggcagcacaagacatctgtcgtagtgcaacaggactaagctcattattctgtaatttgacagcagaattggcccttaaagctgttacaataagaggccattttatcaaaacactctacacgagcacgtgcaggtataattctactacatttatctataggcaaatattttaatgccttctcacatagtgcatcaacagcggcatgagagcaagctgtatacactatgcgagcagaagggtagtagagatctttgaaatttggatctttgtcatccaatttgatggcacctgtgtaggtcaaccacgttcccgaaggtgtgacttccatgccaatgcgcgacattccgaagaacgctgaagcgcacaaaatcctttaggatttggatgatctatgtggcaacggcagtacagacaacacgatgcaccaccaaaggattcttgatccatattggcttccggtgtaactgttattgcctgaccagtaccagtgtgtgtacacaacatcttaacatggcgtggtttgtatgaaatcaccgaaatcataccagttaccattgagatcttgattatctaatgtcagtacaccaacaataccagcatttcgcatggcatcacagaattgtactgtttttaacaaagcttggcgtacacgttcacctaaaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattgcacagaattttgagcagtttcaagagtgcgggagaaaattgatcgtacaacacgagcagcctctgatgcaaatgcataaagaggactcagtattgatttctgttcaccaatattccaggcaccttttttagcttttccttttgtaatctccctatagtgagtcgtattagcttagggtaccgcggacaagacacgtggccttgacggccttccgc

SEQ ID NO: 46 - DNA, 357 bp, Homo sapiens
tggcacgacaggtttcccgactggaaagcgggcagtgagcggaaggcccatgaggcccaggcagttacgcgtgcaactcacggtgagcggctgtctccacaagtccgcgcagagccttcaggtcagaacccgctcgcaggtccaaatctgcaaacaccgccatacccttgctagtaggtgtagattctcgaaaagtcttttagtaggtgatcctctggtacgtcatataatatatctgctctatatagccacttccacgcttagatcttctccctatagtgagtcgtattagcttagatgcggccttgacggccttccgccaattcgccctatagtgagtcgtattacgtcgcgctc

SEQ ID NO: 47 - RNA, 1466 nt, Severe acute respiratory syndrome coronavirus 2
gagcggaaggcccaugaggccagucuugugcuccgagcucgcaguuacgcgucccuagucucagaccuucccaagggacaugggaguggagugacaggacgcacucagcucguggccccacugaugagcuucccuccgcauguaucuauacccuugcuaguagguguagauucucgaaaagucuuuuaguaggugauccucugguacgucauauaauauaucugcucuauauagccacuuccacgcuuagaucuguagaagccuuuuggcaauguuguuccuugaggaaguuguagcacgauugcagcauuguuagcaggauugcgggugccaaugugaucuuuugguguauucaaggcucccucaguugcaacccauaugaugccguuguguuguaguaagcuaacgcauugucaucagugcaagcaguuuguguaguaccggcagcacaagacaucugucguagugcaacaggacuaagcucauuauucuguaauuugacagcagaauuggcccuuaaagcuguuacaauaagaggccauuuuaucaaaacacucuacacgagcacgugcagguauaauucuacuacauuuaucuauaggcaaauauuuuaaugccuucucacauagugcaucaacagcggcaugagagcaagcuguauacacuaugcgagcagaaggguaguagagaucuuugaaauuuggaucuuugucauccaauuugauggcaccuguguaggucaaccacguucccgaaggugugacuuccaugccaaugcgcgacauuccgaagaacgcugaagcgcacaaaauccuuuaggauuuggaugaucuauguggcaacggcaguacagacaacacgaugcaccaccaaaggauucuugauccauauuggcuuccgguguaacuguuauugccugaccaguaccaguguguguacacaacaucuuaacauggcgugguuuguaugaaaucaccgaaaucauaccaguuaccauugagaucuugauuaucuaaugucaguacaccaacaauaccagcauuucgcauggcaucacagaauuguacuguuuuuaacaaagcuuggcguacacguucaccuaaaccacgaauucgucugguagcucuucgguaguagccaauuuggucaucuggacugcuauugguguuaauuggaacgccuuguccucgagggaauuuaaggucuuccuugccauguugagugagagcggugaaccaagacgcaguauugcacagaauuuugagcaguuucaagagugcgggagaaaauugaucguacaacacgagcagccucugaugcaaaugcauaaagaggacucaguauugauuucuguucaccaauauuccaggcaccuuuuuuagcuuuuccuuuuguaaucucccuauagugagucguauuagcuuaggguaccgcggacaagacacguggccuugacggccuuccgc

SEQ ID NO: 48 - RNA, 357 nt, Homo sapiens
uggcacgacagguuucccgacuggaaagcgggcagugagcggaaggcccaugaggcccaggcaguuacgcgugcaacucacggugagcggcugucuccacaaguccgcgcagagccuucaggucagaacccgcucgcagguccaaaucugcaaacaccgccauacccuugcuaguagguguagauucucgaaaagucuuuuaguaggugauccucugguacgucauauaauauaucugcucuauauagccacuuccacgcuuagaucuucucccuauagugagucguauuagcuuagaugcggccuugacggccuuccgccaauucgcccuauagugagucguauuacgucgcgcuc

SEQ ID NO: 49-50 - skipped (2 entries)

SEQ ID NO: 51 - DNA, 2001 bp, Severe acute respiratory syndrome coronavirus 2
tgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctgg

SEQ ID NO: 52 - DNA, 2001 bp, Severe acute respiratory syndrome coronavirus 2
tgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtg

SEQ ID NO: 53 - DNA, 1284 bp, Severe acute respiratory syndrome coronavirus 2
tgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcaga

(19) United States
(12) Patent Application Publication
(10) Pub. No.: US 2025/0333806 A1
(43) Pub. Date: Oct. 30, 2025

(54) COMPOSITIONS, KITS, AND METHODS FOR VARIANT-RESISTANT DETECTION OF TARGET VIRAL SEQUENCES

(71) Applicant: LIFE TECHNOLOGIES CORPORATION, Carlsbad, CA (US)

(72) Inventors: Chunling WANG, Redwood City, CA (US); Yulei SHANG, San Francisco, CA (US); Namrata PABBATI, Fremont, CA (US); Kelly LI, Redwood City, CA (US); Ioanna PAGANI, Oro Valley, AZ (US); Pius BRZOSKA, Woodside, CA (US); Michael TANNER, Highland, CA (US); Mark SHANNON, San Francisco, CA (US); Noah ELDER, Lafayette, CA (US); Kalpith RAMAMOORTHI, San Jose, CA (US); Jason LA, San Bruno, CA (US); Daniel BLANCHARD, South San Francisco, CA (US); Jisheng LI, Pleasanton, CA (US)

(21) Appl. No.: 18/549,134

(22) PCT Filed: Jan. 18, 2022
(86) PCT No.: PCT/US2022/012805

§ 371 Date: Sep. 5, 2023

Related U.S. Application Data
(60) Provisional application No. 63/200,709, filed on Mar. 23, 2021.
(60) Provisional application No. 63/201,059, filed on Apr. 9, 2021.

(51) Int. Cl. C12Q 1/70; C12Q 1/6825; C12Q 1/6844

(52) U.S. Cl. CPC C12Q 1/701; C12Q 1/6825; C12Q 1/6844

(57) ABSTRACT

Disclosed are compositions, assays, methods, diagnostic methods, kits and diagnostic kits for the specific and differential detection of SARS-CoV-2, including SARS-CoV-2 variants, or other coronaviruses from samples including veterinary samples, clinical samples, food samples, forensic sample, an environmental sample (e.g., soil, dirt, garbage, sewage, air, or water), including food processing and manufacturing surfaces, or a biological sample.

### CROSS-REFERENCE TO RELATED APPLICATIONS

This application claims priority to and the benefit of: U.S. Provisional Patent No. 63/200,709, filed Mar. 23, 2021; and U.S. Provisional Patent No. 63/201,059, filed Apr. 9, 2021. Each of the foregoing references is incorporated herein in its entirety by this reference.

### INCORPORATION BY REFERENCE OF SEQUENCE LISTING

This application includes a Sequence Listing submitted electronically in ASCII format. The ASCII copy of the Sequence Listing, created on Jan. 13, 2022, is named LT01620PCT-11398.269a-SL.txt and is 26,681 bytes in size. The ASCII copy of the Sequence Listing is expressly incorporated herein by this reference.

### FIELD

The present teachings relate to compositions, methods, systems and kits for specific detection, diagnosis and differentiation of viruses involved in infectious diseases. Differential detection of specific viral agents allows accurate diagnosis so that appropriate treatment and infection control measures can be provided in a timely manner.

### BACKGROUND

Assays to detect target nucleic acid sequences of interest are widely used in molecular biology and medicine. Many such assays can be sensitive to the presence of mutations or variations in the target nucleic acid sequence and performance of such assays may be reduced or completely eliminated in the presence of such mutant or variant target nucleic acid sequences. For example, genetic assays frequently utilize sequence-specific binding or hybridization between two or more nucleic acid molecules, often with a subsequent step of nucleotide polymerization prior to detection. Such binding or hybridization can be reduced or absent when one or more mutations or variations are present in the target nucleic acid sequence, and the mutant or variant version of the target nucleic acid sequence will remain undetected in the assay.

In some embodiments, the disclosure relates to compositions, methods, and kits to detect target nucleic acid sequence(s) of interest, irrespective of the presence of one or more mutations or variants in the target nucleic acid sequence(s). Optionally, such compositions, methods, and kits involve the use of “redundant” assays, (e.g., multiple different assays directed to different regions of the same target gene, or alternatively multiple different assays directed to a set of multiple target sequences) that, in at least some embodiments, are all detectable using the same detection mode (e.g., one or more dyes all detectable in the same detection channel, optionally at the same or similar wavelength). The use of redundant assays in this manner ensures that a particular target sequence of interest will be detected as present, even when one or more of the redundant assays is ineffective due to the presence of a mutation or other variation that undermines performance of that specific redundant assay. In such a situation, the presence of alternative assays directed to the same target (or set of targets) compensates for the deficiency in one or more of the assays and the target is still detected. In some embodiments, the redundant assays are directed to different target regions within the same target gene or organism. In some embodiments, the disclosed compositions and methods can be used in a system that includes multiple detection channels, with multiple groups of redundant assays, each group of redundant assays targeting a particular target nucleic acid sequence of interest (e.g., a target gene or other sequence) and each group being detectable in one of the detection channels. In some embodiments, the redundant assays are directed to different members of a target group of genes or of a target group of organisms (e.g., gram negative or gram-positive bacteria, a group of coronaviruses, a group of influenza viruses, and the like).

In some embodiments, the methods, compositions, and kits of the disclosure are useful in detection of multiple different variants of a single target organism. In exemplary embodiments, the disclosure relates to compositions, kits, and associated methods involving redundant assays to detect the presence of coronaviruses in a sample. Coronaviruses are a family of viruses having a positive-sense single stranded RNA genome of about 30 kilobases in length. Human coronaviruses were first identified in the mid 1960's as being one of the many etiologic agents of the common cold. People around the world commonly get infected with human coronavirus strains 229E (an alpha coronavirus), NL63 (an alpha coronavirus), OC43 (a beta coronavirus), and HKU1 (a beta coronavirus). These infections present with mild clinical symptoms and are associated with an extremely low mortality rate.

Some coronaviruses infect non-human animals where they can evolve and undergo zoonosis, expanding their tropism to humans. Such crossover events have proven devastating in years past. For example, the Middle East Respiratory Syndrome (MERS) was caused by MERS-CoV, a beta coronavirus that crossed over from dromedary camels to humans. MERS-CoV was associated with a high mortality rate of approximately 35%, but its low transmissibility rate helped to limit its spread and potential for devastation. As another example, Severe Acute Respiratory Syndrome (SARS), which was caused by SARS-CoV, another beta coronavirus, was believed to have been transmitted from bats to civet cats who then transmitted the virus to humans. Although not as deadly as MERS-CoV, SARS-CoV was nevertheless associated with a moderately high mortality rate of approximately 9.6%. Likely due, at least in part, to the lifecycle of SARS-CoV within humans, the spread of this virus was limited mostly to Southeast Asian countries. Human infected with SARS-CoV often became symptomatic prior to shedding infectious virions, making quarantining a particularly useful tool for limiting exposure and spread of the infection.

More recently, a new variant beta coronavirus, SARS-CoV-2 (also known as 2019-nCoV), has emerged, potentially from a crossover event between bats or pangolins and humans in Wuhan, China. While the epidemiological data are incomplete, reports so far indicate that nearly 317 million people worldwide are believed to have been infected by SARS-CoV-2. However, unlike MERS-CoV and SARS-CoV before it, SARS-CoV-2 appears to be significantly less lethal on average with a mortality rate of about 2%. Due to its increased transmissibility, the seemingly small percentage of deaths associated with SARS-CoV-2 belies its worldwide impact, having caused an estimated 5.51 million deaths, at the time of this writing, in the worldwide pandemic. The raw number of humans impacted by SARS-CoV-2 dwarfs the total number of deaths caused by MERS-CoV and SARS-CoV combined—reportedly around 1,600.

Further, because SARS-CoV-2 is an RNA virus, it can mutate with relatively high frequency, with some estimating that SARS-CoV-2 undergoes about 1-2 mutations per month. Some variants, however, have acquired mutations more rapidly than expected. Indeed, as the pandemic has progressed, multiple new mutations and variants have been identified. The term “variant” is used to describe a subtype of a microorganism that is genetically distinct from a major “reference” form. SARS-CoV-2 variants are designated according to the Pango lineage nomenclature system, and more recently have also been identified using a World Health Organization (WHO) label. For example, for much of 2021 the dominant variant of SARS-CoV-2 in the United States and most of the world was the B.1.617.2 variant (under the Pango lineage nomenclature), more commonly referred to as “the Delta variant” (under the corresponding WHO label). At the time of this writing, the dominant variant is the B.1.1.529 variant (under the Pango lineage nomenclature), more commonly referred to as “the Omicron variant”.

Given the present and continuing emergence of new and/or variant coronaviruses, there is an urgent need to develop compositions, kits, and methods that are robust in detecting the presence of SARS-CoV-2 nucleic acid in a sample even in circumstances where the sample contains one or more existing or future SARS-CoV-2 mutant variants. Accurate, robust assays are needed so that appropriate treatment and infection control measures can be properly instituted in a timely manner, unhampered by excessive risk of false negatives and/or a lack of confidence in conventional assays. In particular, given that SARS-CoV-2 is expected to continue to mutate and develop new variants as the pandemic progresses, there is an urgent need to develop assays capable of effectively detecting the presence of SARS-CoV-2 despite known and future mutations.

Accordingly, there are a number of disadvantages with current compositions, kits, and methods for detecting SARS-CoV-2, particularly as new mutations and variants continue to emerge, that are addressed by the compositions, kits, and methods disclosed herein.

### BRIEF DESCRIPTION OF THE DRAWINGS

FIGS. 1A and 1B illustrate the sequence identity between SARS-CoV-2 and three closely related coronaviruses, namely, Bat-SL-CoVZC45, Bat-SL-CoVZXC21 and SARS-CoVGZO2. (Taken from Lu et al. “Genomic characterization and epidemiology of 2019 novel coronavirus: implications for virus origins and receptor binding.” Roujian Lu et al., The Lancet, Elsevier, Available online 30 Jan. 2020).

FIG. 2A is a schematic diagram of the RNA genome of SARS-CoV-2, illustrating potential target genes to which assays described herein may be targeted.

FIG. 2B is a schematic illustration of the SARS-CoV-2 virion structure.

FIG. 3 illustrates exemplary assays, including associated primers and probes that may be utilized for the robust, variant-resistant identification of SARS-CoV-2 within a sample.

FIGS. 4A and 4B illustrate amplification plots resulting from a process using exemplary variant-resistant assay described herein. FIGS. 4A and 4B show effective amplification of multiple different SARS-CoV-2 target regions (e.g., a multiplex reaction) using a panel or combination of assays which provide redundancy by targeting more than one target region within a particular target gene.

FIGS. 5 and 6 illustrate plasmid maps according to some embodiments.

### DETAILED DESCRIPTION

### Introduction

All publications and patent applications cited herein are incorporated by reference in their entirety for all purposes to the same extent as if each individual publication or patent application were specifically and individually indicated to be so incorporated by reference. Further, although the present invention has been described in some detail by way of illustration and example for purposes of clarity and understanding, it will be apparent that certain changes and modifications may be practiced within the scope of the spirit and substance of this disclosure and of the appended claims.

The term “variant-resistant”, as used herein, refers to the property of the compositions, kits, and methods of the present disclosure of being capable of accurately detecting the presence of target genes or target organisms within a sample over a broad range of potential mutations and variants. In other words, where a conventional assay may fail to detect the presence of a particular target organism in a sample (e.g., because of the presence of mutations in the target organism), and thereby result in a false negative, the compositions, kits, and methods of the present disclosure include multiple layers of redundancy that increase the likelihood that at least some portion of the target organism's nucleic acid within the sample will be detected. These multiple layers of redundancy are described in more detail below. One exemplary and non-limiting method that may be utilized to illustrate the “variant-resistant” property of the disclosed embodiments includes comparing the detection rate of an assay disclosed herein against a conventional assay (created prior to this disclosure) using a particular SARS-CoV-2 variant or a panel of different SARS-CoV-2 variants (including synthetic variants that have been engineered to include mutations or other genetic variations). In some embodiments, the “variant-resistant” nature of the disclosed assays can thus be shown by measuring higher accuracy (and in particular a lower false negative rate) as compared to such conventional assays.

In addition, although many of the examples described herein are particular to assays and methods for the detection of SARS-CoV-2 within a sample, the components, processes, and features described herein are also applicable to other applications and other target organisms or genes. For example, the embodiments described herein may be readily applied, or appropriately modified, for use in detecting other genetic sequences of interest, including other (non-SARS-CoV-2) coronavirus sequences, other viruses associated with respiratory infection, other viruses of interest, and/or other non-viral organisms. As used herein, “organism” refers to any entity containing nucleic acid and that is capable of supporting replication of such nucleic acid, including but not limited to any unicellular or multicellular lifeform, prokaryotic or eukaryotic, as well as phages and virions, even though phages and virions are incapable of self-replication without an infected host.

Given the present and continuing emergence of new genetic mutations and variants of interest and the importance of understanding the biological impact of such mutations in various contexts (such as, for example, tracking and diagnosis of the presence of infectious organisms, cancer-associated mutations, genealogy, and the like), there is an urgent need to develop compositions, kits, and methods for accurate detection and characterization of genetically variable targets. In the case of SARS-CoV-2, for example, such compositions, kits and methods would support institution of appropriate treatment and infection control measures in a timely manner, without excessive risk of false negative results and the concomitant lack of confidence in control measures. Furthermore, it is desirable that genetic assays to detect the presence of nucleic acid targets of interest remain accurate and relevant despite the emergence of mutant or variant forms of such nucleic acid targets, thereby avoiding the need for redesign and/or fresh validation (and, where applicable, separate regulatory approval) of such assays. Continuing with the non-limiting example of SARS-CoV-2, each misidentified or misdiagnosed instance of SARS-CoV-2 infection further convolutes the epidemiological data and prevents the implementation of appropriate, informed solutions that may help reign in the pandemic. For example, missed diagnoses may be related to the failure of present detection assays to properly detect the presence of SARS-CoV-2 nucleic acid within a sample because the SARS-CoV-2 has particular mutations that reduce the accuracy of the detection assay.

In some embodiments, the present disclosure relates to compositions, kits, and methods for detection of coronaviruses, in particular the coronavirus SARS-CoV-2. In some embodiments, the compositions, kits, and methods disclosed herein are designed to provide robust and accurate detection of SARS-CoV-2 nucleic acid within a sample, even if the SARS-CoV-2 nucleic acid includes mutations and/or is associated with a variant that otherwise results in a high false negative rate using conventional detection assays. When an example “embodiment” or a particular “assay” is described herein, it will be understood that the features of the embodiment may be applicable to a composition (e.g., the particular physical components of an assay such as primers and/or probes), a kit (e.g., primers and/or probes and additional buffers, reagents, etc.), or a method (e.g., a process for detecting target nucleic acids) as appropriate. For simplicity, many embodiments are presented by describing “assays”, but it will be understood that the associated methods of using the assays are also intended to form part of this disclosure.

The SARS-CoV-2 virus, also known as 2019-nCoV, is associated with the human respiratory disease COVID-19. The virus isolated from early cases of COVID-19 was provisionally named 2019-nCoV. The Coronavirus Study Group of the International Committee on Taxonomy of Viruses has subsequently given the official designation of SARS-CoV-2. For the purposes of this disclosure SARS-CoV-2 and 2019-nCoV are considered to refer to the same virus.

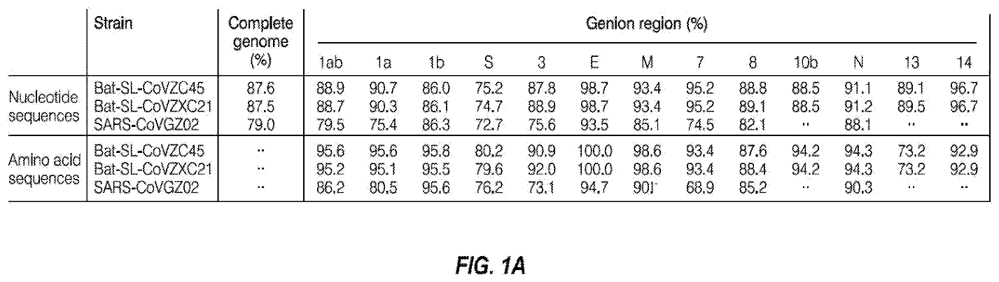

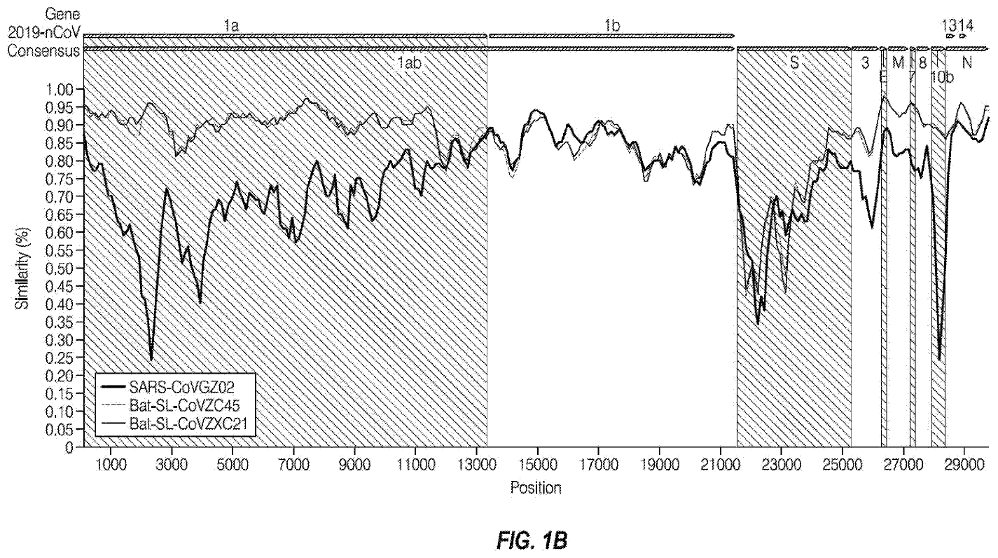

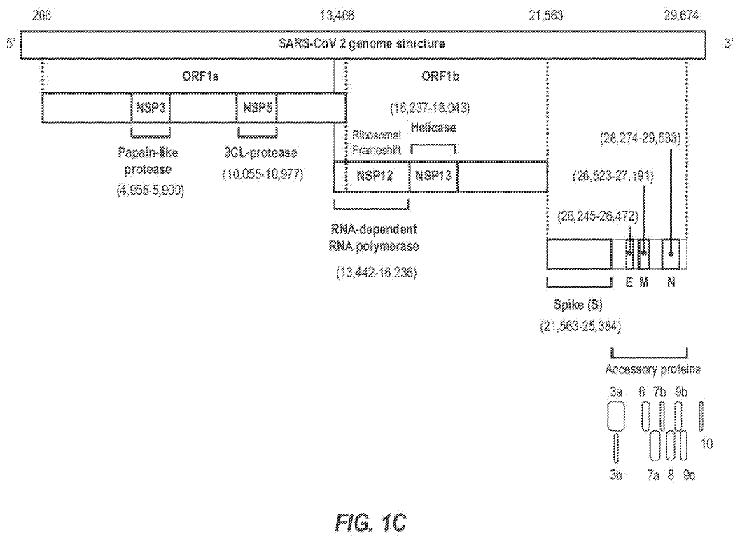

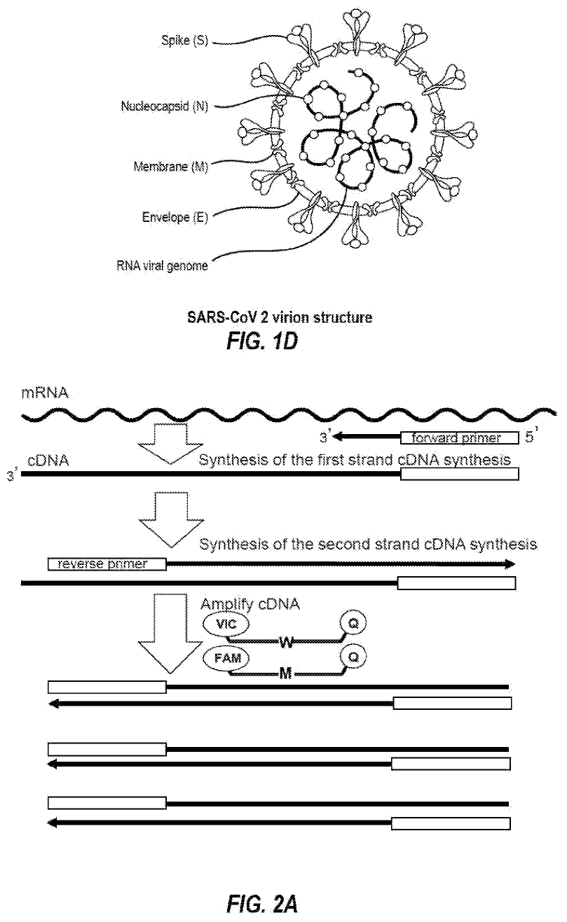

Initial genetic characterization SARS-CoV-2 was reported by Lu et al. (“Genomic characterization and epidemiology of 2019 novel coronavirus: implications for virus origins and receptor binding.” Roujian Lu et al., The Lancet, Elsevier, Available online 30 Jan. 2020). Lu identified three coronavirus that show close homology to SARS-CoV-2: Bat-SL-CoVZC45, Bat-SL-CoVZXC21 and SARS-CoVGZO2. The sequence identity between these strains is depicted in FIGS. 1A and 1B. This analysis identified three genetic regions with significant variability between SARS-CoV-2 and the other viruses, specifically in the viral genes encoding the viral proteins for an ORF protein (e.g., ORF1a, ORF1b, ORF1ab, ORF8), the S protein and the N protein.

The genetic sequence of this “reference” form of SARS-CoV-2 is based on the sequence associated with NCBI accession no. NC_045512.2. (see also GenBank: MN908947.3) which describes a genome of 29,903 base pairs. As an example of certain regions of the “reference” form, the region of bp 1000 to bp 3000, associated with Orf1ab, is shown in Table 1.

TABLE 1
(bp 1 corresponds to bp 1000 of MN908947) SEQ ID NO: 51.

| | |
|---|---|
| 1 | tgaaaagagc tatgaattgc agacaccttt tgaaattaaa ttggcaaaga aatttgacac |
| 61 | cttcaatggg gaatgtccaa attttgtatt tcccttaaat tccataatca agactattca |
| 121 | accaagggtt gaaaagaaaa agcttgatgg ctttatgggt agaattcgat ctgtctatcc |
| 181 | agttgcgtca ccaaatgaat gcaaccaaat gtgcctttca actctcatga agtgtgatca |
| 241 | ttgtggtgaa acttcatggc agacgggcga ttttgttaaa gccacttgcg aattttgtgg |
| 301 | cactgagaat ttgactaaag aaggtgccac tacttgtggt tacttacccc aaaatgctgt |
| 361 | tgttaaaatt tattgtccag catgtcacaa ttcagaagta ggacctgagc atagtcttgc |
| 421 | cgaataccat aatgaatctg gcttgaaaac cattcttcgt aagggtggtc gcactattgc |
| 481 | ctttggaggc tgtgtgttct cttatgttgg ttgccataac aagtgtgcct attgggttcc |
| 541 | acgtgctagc gctaacatag gttgtaacca tacaggtgtt gttggagaag gttccgaagg |
| 601 | tcttaatgac aaccttcttg aaatactcca aaaagagaaa gtcaacatca atattgttgg |
| 661 | tgactttaaa cttaatgaag agatcgccat tattttggca tctttttctg cttccacaag |
| 721 | tgcttttgtg gaaactgtga aaggtttgga ttataaagca ttcaaacaaa ttgttgaatc |
| 781 | ctgtggtaat tttaaagtta caaaaggaaa agctaaaaaa ggtgcctgga atattggtga |
| 841 | acagaaatca atactgagtc ctctttatgc atttgcatca gaggctgctc gtgttgtacg |
| 901 | atcaattttc tcccgcactc ttgaaactgc tcaaaattct gtgcgtgttt tacagaaggc |
| 961 | cgctataaca atactagatg gaatttcaca gtattcactg agactcattg atgctatgat |
| 1021 | gttcacatct gatttggcta ctaacaatct agttgtaatg gcctacatta caggtggtgt |
| 1081 | tgttcagttg acttcgcagt ggctaactaa catctttggc actgtttatg aaaaactcaa |
| 1141 | acccgtcctt gattggcttg aagagaagtt taaggaaggt gtagagtttc ttagagacgg |
| 1201 | ttgggaaatt gttaaattta tctcaacctg tgcttgtgaa attgtcggtg gacaaattgt |
| 1261 | cacctgtgca aaggaaatta aggagagtgt tcagacattc tttaagcttg taaataaatt |
| 1321 | tttggctttg tgtgctgact ctatcattat tggtggagct aaacttaaag ccttgaattt |
| 1381 | aggtgaaaca tttgtcacgc actcaaaggg attgtacaga aagtgtgtta aatccagaga |
| 1441 | agaaactggc ctactcatgc ctctaaaagc cccaaaagaa attatcttct tagagggaga |
| 1501 | aacacttccc acagaagtgt taacagagga agttgtcttg aaaactggtg atttacaacc |
| 1561 | attagaacaa cctactagtg aagctgttga agctccattg gttggtacac cagtttgtat |
| 1621 | taacgggctt atgttgctcg aaatcaaaga cacagaaaag tactgtgccc ttgcacctaa |
| 1681 | tatgatggta acaaacaata ccttcacact caaaggcggt gcaccaacaa aggttacttt |
| 1741 | tggtgatgac actgtgatag aagtgcaagg ttacaagagt gtgaatatca cttttgaact |
| 1801 | tgatgaaagg attgataaag tacttaatga gaagtgctct gcctatacag ttgaactcgg |
| 1861 | tacagaagta aatgagttcg cctgtgttgt ggcagatgct gtcataaaaa ctttgcaacc |
| 1921 | agtatctgaa ttacttacac cactgggcat tgatttagat gagtggagta tggctacata |
| 1981 | ctacttattt gatgagtctg g |

For the S gene region, region bp 21564 thru 23564 is shown in Table 2.

TABLE 2
(bp 1 corresponds to bp 21564 of MN908947) SEQ ID NO: 52.

```
      1 tgaaaagagc tatgaattgc agacaccttt tgaaattaaa ttggcaaaga aatttgacac
    61 cttcaatggg gaatgtccaa attttgtatt tcccttaaat tccataatca agactattca
  121 aagttttcag atcctcagtt ttacattcaa ctcaggactt gttcttacct ttcttttcca
  181 atgttacttg gttccatgct atacatgtct ctgggaccaa tggtactaag aggtttgata
  241 accctgtcct accatttaat gatggtgttt attttgcttc cactgagaag tctaacataa
  301 taagaggctg gatttttggt actactttag attcgaagac ccagtcccta cttattgtta
  361 ataacgctac taatgttgtt attaaagtct gtgaatttca attttgtaat gatccatttt
  421 tgggtgttta ttaccacaaa aacaacaaaa gttggatgga aagtgagttc agagtttatt
  481 ctagtgcgaa taattgcact tttgaatatg tctctcagcc ttttcttatg gaccttgaag
  541 gaaaacaggg taatttcaaa aatcttaggg aatttgtgtt taagaatatt gatggttatt
  601 ttaaaatata ttctaagcac acgcctatta atttagtgcg tgatctccct cagggttttt
  661 cggctttaga accattggta gatttgccaa taggtattaa catcactagg tttcaaactt
  721 tacttgcttt acatagaagt tatttgactc ctggtgattc ttcttcaggt tggacagctg
  781 gtgctgcagc ttattatgtg ggttatcttc aacctaggac ttttctatta aaatataatg
  841 aaaatggaac cattacagat gctgtagact gtgcacttga ccctctctca gaaacaaagt
  901 gtacgttgaa atccttcact gtagaaaaag gaatctatca aacttctaac tttagagtcc
  961 aaccaacaga atctattgtt agatttccta atattacaaa cttgtgccct tttggtgaag
1021 tttttaacgc caccagattt gcatctgttt atgcttggaa caggaagaga atcagcaact
1081 gtgttgctga ttattctgtc ctatataatt ccgcatcatt ttccactttt aagtgttatg
1141 gagtgtctcc tactaaatta aatgatctct gctttactaa tgtctatgca gattcatttg
1201 taattagagg tgatgaagtc agacaaatcg ctccagggca aactggaaag attgctgatt
1261 ataattataa attaccagat gattttacag gctgcgttat agcttggaat tctaacaatc
1321 ttgattctaa ggttggtggt aattataatt acctgtatag attgtttagg aagtctaatc
1381 tcaaaccttt tgagagagat atttcaactg aaatctatca ggccggtagc acaccttgta
1441 atggtgttga aggttttaat tgttactttc ctttacaatc atatggtttc caacccacta
1501 atggtgttgg ttaccaacca tacagagtag tagtactttc ttttgaactt ctacatgcac
1561 cagcaactgt ttgtggacct aaaaagtcta ctaatttggt taaaaacaaa tgtgtcaatt
1621 tcaacttcaa tggtttaaca ggcacaggtg ttcttactga gtctaacaaa aagtttctgc
1681 ctttccaaca atttggcaga gacattgctg acactactga tgctgtccgt gatccacaga
1741 cacttgagat tcttgacatt acaccatgtt cttttggtgg tgtcagtgtt ataacaccag
1801 gaacaaatac ttctaaccag gttgctgttc tttatcagga tgttaactgc acagaagtcc
1861 ctgttgctat tcatgcagat caacttactc ctacttggcg tgtttattct acaggttcta
1921 atgtttttca aacacgtgca ggctgtttaa taggggctga acatgtcaac aactcatatg
1981 agtgtgacat acccattggt g
```

For the N gene region, bp 28275 thru 29558 is shown in Table 3.

TABLE 3
(bp 1 corresponds to bp 28275 of MN908947) SEQ ID NO: 53.

```
      1 tgtctgataa tggaccccaa aatcagcgaa atgcaccccg cattacgttt ggtggaccct
    61 cagattcaac tggcagtaac cagaatggag aacgcagtgg ggcgcgatca aaacaacgtc
  121 ggccccaagg tttacccaat aatactgcgt cttggttcac cgctctcact caacatggca
  181 aggaagacct taaattccct cgaggacaag gcgttccaat taacaccaat agcagtccag
  241 atgaccaaat tggctactac cgaagagcta ccagacgaat tcgtggtggt gacggtaaaa
  301 tgaaagatct cagtccaaga tggtatttct actacctagg aactgggcca gaagctggac
  361 ttccctatgg tgctaacaaa gacggcatca tatgggttgc aactgaggga gccttgaata
  421 caccaaaaga tcacattggc acccgcaatc ctgctaacaa tgctgcaatc gtgctacaac
  481 ttcctcaagg aacaacattg ccaaaaggct tctacgcaga agggagcaga ggcggcagtc
  541 aagcctcttc tcgttcctca tcacgtagtc gcaacagttc aagaaattca actccaggca
  601 gcagtagggg aacttctcct gctagaatgg ctggcaatgg cggtgatgct gctcttgctt
  661 tgctgctgct tgacagattg aaccagcttg agagcaaaat gtctggtaaa ggccaacaac
  721 aacaaggcca aactgtcact aagaaatctg ctgctgaggc ttctaagaag cctcggcaaa
  781 aacgtactgc cactaaagca tacaatgtaa cacaagcttt cggcagacgt ggtccagaac
  841 aaacccaagg aaattttggg gaccaggaac taatcagaca aggaactgat tacaaacatt
  901 ggccgcaaat tgcacaattt gcccccagcg cttcagcgtt cttcggaatg tcgcgcattg
  961 gcatggaagt cacaccttcg ggaacgtggt tgacctacac aggtgccatc aaattggatg
1021 acaaagatcc aaatttcaaa gatcaagtca ttttgctgaa taagcatatt gacgcataca
1081 aaacattccc accaacagag cctaaaaagg acaaaaagaa gaaggctgat gaaactcaag
1141 ccttaccgca gagacagaag aaacagcaaa ctgtgactct tcttcctgct gcagatttgg
1201 atgatttctc caaacaattg caacaatcca tgagcagtgc tgactcaact caggcctaaa
1261 ctcatgcaga ccacacaagg caga
```

As used herein, in the context of SARS-CoV-2 as a target organism, a “mutant” or “variant” has one or more mutations (e.g., SNP or deletion mutations) in one or more of the above regions and/or other sites of the genomic sequence as compared to the “reference” SARS-CoV-2.

Several SARS-CoV-2 qPCR based tests currently on the market are designed to target one or more regions shown in Tables 1-3. Examples include the kit developed by the CDC containing probes targeting the N protein; the kit developed by the Chinese CDC targeting the N and Orf proteins, as well as the WHO kit targeting the N protein, the E protein, and the closely related RdRp SARS/Wuhan coronavirus. However, as discussed above, new mutations and variants of SARS-CoV-2 have emerged and continue to emerge, and the currently available assays are not optimized for such newly emerging variants. The currently available assays may even fail to detect the presence of certain SARS-CoV-2 variants and thus lead to false negative test results. In contrast, the embodiments described herein can be beneficially utilized to better detect SARS-CoV-2 even as new mutations and variants emerge.

Table 4 illustrates some of the mutations that have occurred in the SARS-CoV-2 genome, as well as some of their associated variants, where known. The numbering system used to designate these mutations is based on the “reference” sequence as defined above. For example, the mutation “S.N501Y.AAT.TAT” refers to a mutant form of the spike (S) protein wherein amino acid residue no. 501 is changed from asparagine (A) to tyrosine (Y). The latter portion of the label “AAT.TAT” compares the reference codon to the mutant codon, and in this example illustrates that the mutation is associated with a change from an adenine (A) to a thymine (T) (i.e., the AAT of the reference codon is changed to a TAT in the mutant codon). Note that RNA comprises uracil (U), but notation included herein may sometimes simply refer to the corresponding DNA base pair thymine (T). The initial part of the label specific to the gene or protein involved and/or the latter portion of the label specific to the nucleotide mutation may occasionally be dropped from the label for convenience. The latter portion of the label may also be shortened to simply show the single reference nucleotide and mutant nucleotide, rather than the entire reference and mutant codon. Those with skill in the art will readily recognize the mutation nomenclature used herein.

TABLE 4
SARS-CoV-2 Mutations

| | | | Earliest Documented |
|---|---|---|---|
| Mutation | WHO Label | Associated Variants | Samples |
| S.D215G.GAT.GGT | Beta | B.1.351 | South Africa |
| S.D614G.GAT.GGT | Various | B.1.1.207, P.1, | Nigeria, United |
| | | B.1.1.33, B.1.1.7, | Kingdom, South |
| | | B.1.177, B.1.258, | Africa, Brazil / |
| | | B.1.351, B.1.525, | Amazon |
| | | Mink variant | |
| S.delH69V70 | Alpha | B.1.1.7, B.1.258, | United Kingdom |
| | | B.1.525 | |
| S.delY144 | Alpha | B.1.1.7 | United Kingdom |
| S.E484K.GAA.AAA | Beta, | P.1, B.1.1.33, | South Africa, Brazil |
| | Gamma, | B.1.351, B.1.525 | |
| | Eta | | |
| S.E484Q.GAA.CAA | Delta | B.1.617, B.1.617.1, | India |
| | (absence | B.1.617.3 | |
| | of this | | |
| | mutation) | | |
| S.F888L.TTT.CTT | Eta | B.1.525 | 20A/S: 484K United |
| | | | Kingdom |
| S.K417N.AAG.AAT | Beta | B.1.351 | South Africa |
| S.K417T.AAG.ACG | Gamma | P.1 | Brazil / Amazon |
| S.L18F.CTT.TTT | Gamma | P.1, B.1.351 | South Africa, |
| | | | Brazil/Amazon |
| S.L452R.CTG.CGG | Delta, | B.1.617, B.1.617.1, | India, California |
| | Epsilon | B.1.617.2, B.1.617.3, | |
| | | B.1.429 | |
| S.N439K.AAC.AAA | | B.1.258 | |
| S.N501Y.AAT.TAT | Alpha, | P.1, B.1.1.7, | United Kingdom, South |
| | Beta, | B.1.351 | African, Brazil / |
| | Gamma | | Amazon |
| S.P681H.CCT.CAT | Alpha | B.1.1.207, B.1.1.7 | Nigeria, United |
| | | | Kingdom |
| S.P681R.CCT.CGT | Delta | B.1.617.1, B.1.617.2, | India |
| | | B.1.617.3 | |
| S.S13I.AGT.ATT | Epsilon | B.1.427, B.1.429 | California |
| S.S477N.AGC.AAC | Iota | B.1.526 | New York |
| S.T20N.ACC.AAC | Gamma | P.1 | Brazil / Amazon |
| S.V1176F.GTT.TTT | | P.2 | Brazil |
| Orf1ab.A13057T | Mu | B.1.621 | Colombia |
| Orf1.A1708D.GCT.GAT | Alpha | B.1.1.7 | United Kingdom |
| S.A222V.GCT.GTT | | B.1.177 | |
| Orf1ab.A2710T | Omicron | B.1.1.529 | Various |
| CoV1.A28272T | Mu | B.1.621 | Colombia |
| S.A570D.GCT.GAT | Alpha | B.1.1.7 | United Kingdom |
| S.A701V.GCA.GTA | Beta | B.1.351 | South Africa |
| N.D3L.GAT.CTA | | | |
| S.D80A.GAT.GCT | Beta | B.1.351 | South Africa |
| S.delL242.244L | Beta | B.1.351 | South Africa |
| S.E583D.GAG.GAT | | B.177 | |
| S.E583D.GAG.GAC | | B.177 | |
| S.EFR156-158G | Delta | B.1.617.2 | India |
| S.F157L.TTC.TTA | | A.23 | |
| S.F157S.TTC_TCC | Iota | B.1.526 | United States |
| S.G339D.GGT_GAG | Omicron | B.1.1.529 | Various countries |
| S.G446V.GGT.GTT | | | |
| S.G769V.GGA.GTA | | | |
| S.H66D.CAT.GAT | | | |
| ORF1.K1795Q.AAA.CAA | | | |
| S.L452Q.CTG.CAG | Lambda | C.37 | Mink |
| S.N450K.AAT.AAG | | B.1.214 | |
| S.N460K.AAT.AAA | | | |
| S.N460K.AAT.AAG | | | |
| S.N460T.AAT.ACT | | | |
| P5743S.CCA_TCA | Mu | B.1.621 | Colombia |
| orf8.Q27ST.CAA.TAA | Alpha | B.1.1.7 | United Kingdom |
| S.Q493R.CAA_CGA | Omicron | B.1.1.529 | Various countries |
| S.Q498R.CAA_CGA | | | |
| S.Q52R.CAG.CGG | Eta | 1.525 | |
| S.Q613H.CAG.CAT | | A.23 | |
| S.Q677H.CAG.CAT | Eta | 1.525 | |
| S.Q677H.CAG.CAC | Eta | 1.525 | |
| S.R246I.AGA.ATA | Beta | B.1.351 | South Africa |
| S.R346K.AGA_AAA | Mu | B.1.621 | Colombia |
| S.S982A.TCA.GCA | Alpha | B.1.1.7 | United Kingdom |
| S.Y145H.TAC.CAC | Delta+ | AY.4.2 | India |
| T1055A.ACA_GCA | Mu | B.1.621 | Colombia |
| Orf1ab.T13195C | Omicron | B.1.1.529 | Various |
| S.T19R.ACA.AGA | Delta | B.1.617.2 | India |
| S.T478K.ACA.AAA | Delta | B.1.617.2 | |
| S.T547K.ACA.AAA | Omicron | B.1.1.529 | Various |
| S.T716I.ACA.ATA | Alpha | B.1.1.7 | United Kingdom |
| S.T95I.ACT_ATT | Iota, Mu | B.1.526, B.1.621.1 | United States, |
| | | | Colombia |
| S.V367F.GTC.TTC | | A.23 | |
| S.W152L.TGG.TTG | | R.1 | |
| S.Y453F.TAT.TTT | | B.1.1.298 | Mink |
| S.D1118H.G_C | | B.1.1.7 | United Kingdom |

As explained above, these mutant variants, as well as others that may emerge in the future, may not be detected with the same efficacy using conventional diagnostic assays.

### Redundancy of Target Genes

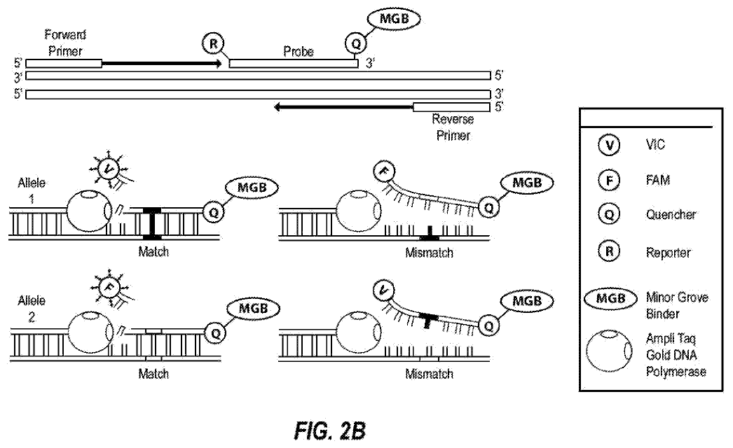

Because SARS-CoV-2 is an RNA virus, it can mutate with relatively high frequency, meaning additional mutations and variants will continue to emerge over time. FIG. 2A is a diagram of the SARS-CoV-2 RNA genome showing particular regions that may be targeted. As shown, potential target genes include the Orf1a, Orf1b, S, E, M, and N genes, among several other accessory proteins. The SARS-CoV-2 genome encodes two large genes Orf1a and Orf1b, which encode 16 non-structural proteins (NSP1-NSP16). These NSPs are processed to form a replication-transcription complex (RTC) that is involved in genome transcription and replication. With additional reference to FIG. 2B, which illustrates the SARS-CoV-2 virion structure, the structural genes encode the structural proteins, spike (S), envelope (E), membrane (M), and nucleocapsid (N). The accessory proteins are unique to SARS-CoV-2 in terms of number, genomic organization, sequence, and function.

Applicants have found that accurate detection of SARS-CoV-2 can be promoted, even in the case of existing or future variants, by targeting multiple regions of the SARS-CoV-2 genome, thereby compensating for possible virus mutations and variants. For example, some embodiments are designed to target one or more regions within a first target gene and one or more target regions within a second target gene. Some embodiments may additionally target one or more target regions within a third target gene, fourth target gene, or more (e.g., sixth, seventh, eighth, ninth, tenth, eleventh, etc.) target genes. In one embodiment, an assay is formulated to target one or more regions within one of the Orf1a, Orf1b, or N genes, and to additionally target one or more regions within a separate one of the Orf1a, Orf1b, or N genes. For example: an assay may be formulated to target one or more regions within the Orf1a gene and separately target one or more regions within the Orf1b gene; an assay may be formulated to target one or more regions within the Orf1a gene and separately target one or more regions within the N gene; an assay may be formulated to target one or more regions within the Orf1b gene and separately target one or more regions within the N gene. In one embodiment, an assay is formulated to target one or more regions in each of the Orf1a, Orf1b, and N genes. Additionally, or alternatively, an assay may be formulated with one or more target regions associated with the S, E, or M gene, or with an accessory protein gene.

In some embodiments, positive identification of SARS-CoV-2 (or of specific target organism(s) or target gene(s) of interest) may be determined by detection of at least one target gene using redundant assays. In some embodiments, multiple target genes may be detected using redundant assays. In some exemplary embodiments focusing on detection of SARS-CoV-2, positive identification of SARS-CoV-2 may be determined by detection of at least two target genes from the SARS-CoV-2 genome. In some embodiments, positive identification of SARS-CoV-2 may be determined by detection of at least three target genes. In some embodiments, positive identification of SARS-CoV-2 is determined by detection of at least one of an Orf1a gene target, Orf1b gene target, and/or N gene target. In some embodiments, positive identification of SARS-CoV-2 is determined by detection of at least two of an Orf1a gene target, Orf1b gene target, and/or N gene target. In some embodiments, positive identification of SARS-CoV-2 is determined by detection of each of an Orf1a gene target, Orf1b gene target, and N gene target. In some embodiments, positive identification of SARS-CoV-2 is additionally or alternatively determined by detection of one or more of the S, E, or M gene, or an accessory protein gene. In some implementations where an assay is configured to detect the presence of multiple target genes and only a subset is detected, the methods as described herein can further include confirmation by Sanger sequencing for determination of a positive diagnosis of SARS-CoV-2 and/or for specifying the mutations/variant involved.

Redundancy of Target Regions within Target Genes

In addition to the redundancies leveraged through the use of genetic assays designed to interrogate multiple target genes, at least some embodiments described herein provide additional layers of redundancy by targeting more than one region within a particular target gene. For example, an assay may include a first forward primer and a first reverse primer for generating a first amplification product of a first target region if said first target region is present in the sample, and a second forward primer and second reverse primer for generating a second amplification product of a second target region if said second target region is present in the sample, where the first target region and the second target region are both in the same target gene. In some embodiments, an assay may be targeted to more than two target regions within a target gene, and may therefore include components targeting multiple (third, fourth, etc.) target regions within a particular target gene.

Utilizing multiple target regions, even within the same target gene, decreases the risk of false negatives resulting from mutations within the target gene. For example, even if mutations associated with a first target region are significant enough to meaningfully reduce detection of the first target region, it is likely that the second target region of the gene can still be effectively detected. Thus, overall, the assay remains capable of detecting a target gene even in circumstances where mutations have otherwise resulted in significant portions of the target gene failing to amplify. In some embodiments, some or all of the multiple target regions within one or more target genes do not overlap with each other, thereby spreading the assay coverage across the target gene and reducing the likelihood that multiple (e.g., two or more) target regions will fail in the face of future mutations.

In a further example, an embodiment may include components that enable detection of multiple target genes each of which include multiple target regions for detection. For example, in addition to a first and/or a second target region of the first target gene, an assay may be further formulated to target third and/or fourth target regions, where the third and fourth target regions are present within a second target gene. Moreover, an assay may further include components that enable detection of fifth and/or sixth target regions, where the fifth and sixth target regions are present within a third target gene. As explained above, an assay may provide for detection of more than two target regions within any or all of the target genes. Thus, for example, an assay may further include components that enable detection of a seventh target region (e.g., present within the first target gene) and/or an eighth target region (e.g., present within the second target gene).

Some assays may include additional components for detection of additional target regions (e.g., within the first, second, and/or third target gene) and/or for detection of additional target genes (e.g., fourth, fifth, sixth, etc.) target genes. Thus, for example, an assay may further include components that enable detection of a ninth target region and/or tenth target region, where one or both are in a fifth target gene.

In some embodiments one or more target regions are associated with a positive control. As an example, the ninth and/or tenth target regions may be associated with a positive control sequence such as a human RNase P or bacteriophage MS2 sequence. In some embodiments, therefore, at least one of the first, second, third, fourth, fifth, sixth, seventh, and/or eighth target regions are within a first target organism, and the ninth and/or tenth target regions are present within a second target organism. The first target organism may be a virus (e.g., SARS-CoV-2), while the second target organism may be the organism from which a control sequence was sourced (e.g., human or bacteria). In other embodiments, an assay may provide for detection of target regions associated with more than two different organisms (e.g., three, four, five organisms, etc.). An assay may be directed to detection of a panel of pathogenic organisms, for example.

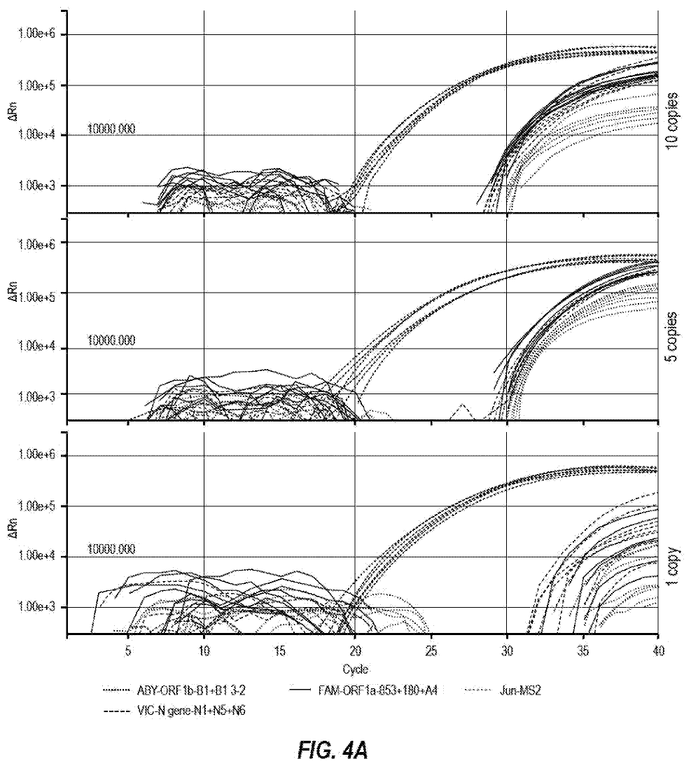

Using the assays disclosed herein, a limit of detection (LoD) of any single assay or combination (e.g., a panel) of assays may be established. For example, in some embodiments, a LoD of 20 or less copies of virion copies per reaction (e.g., ≤20 copies/rxn, 15 copies/rxn, 10 copies/rxn, 5 copies/rxn, 2 copies/rxn, 1 copy/rxn) can be detected. In some embodiments, the LoD of an assay is generally considered the lowest concentration of target that can be reliably detected over a number of repeated measurements. In some other embodiments, the LoD may also be used as a measure of assay sensitivity. In some embodiments, LoD values are reported in units other than copies of viral genomic RNA per microliter or virion or viral copies per microliter (copies/mL), such as copies/μL, TCID50, copies per reaction or copies per reaction volume, genomic copy equivalents (GCE) per reaction, or molarity of assay target. In some embodiments, LoD (e.g., viral copies/rxn) can be determined as described in Examples 2 and 3 (see FIGS. 4A and 4B). In some embodiments, LoD can be determined as described in Arnaout, et al.; doi: https://doi.org/10.1101/2020.06.02.131144, the disclosure of which is incorporated by reference in its entirety. In some other embodiments, LoD can be determined based on the current standard protocols and/or guidance provided by the FDA (e.g., for EUA approval). In some embodiments, the assays as disclosed herein, provide a LoD of 10 copies or less/reaction. In some embodiments, the assays as disclosed herein, provide a LoD of 5 copies or less/reaction. In some embodiments, the assays as disclosed herein, provide a LoD of 1 copy/reaction.

### Example Assays & Associated Components

Embodiments disclosed herein include primers and optionally probes useful for the detection of SARS-CoV-2 in a sample (e.g., a biological or environmental sample). Such primers and probes can be used in a nucleic acid assay (singleplex or multiplex) for detection and identification one or more nucleic acid targets in a sample. The singleplex and multiplex assays described herein demonstrate a high level of sensitivity, specificity, and accuracy, and are particularly robust in detecting the presence of SARS-CoV-2 despite mutations and variants of SARS-CoV-2.

In some embodiments, assays are configured to detect an amplification product of a particular target region by detecting a signal from a label (i.e., a detectable label) or other signal-generating process, where the signal indicates formation of the amplification product. In some embodiments, the label is attached to, or otherwise associated with, the corresponding forward primer and/or reverse primer used to generate the amplification product. Additionally, or alternatively, the label is attached to, or otherwise associated with, a probe configured to associate with a probe binding sequence within the target region. In some embodiments, the label is an optically detectable label. Alternatively, the label may be detectable via non-optical means including electronically, electrically, or using NMR, sound, radioactivity, and the like.

An assay may be configured to detect a target nucleic acid sequence of interest using a single detection channel or multiple detection channels. Each target region from a target nucleic acid sequence of interest may have its own detection channel. Alternatively, at least one detection channel may be associated with multiple target regions. For example, a first label associated with a primer and/or probe of a first target region may be configured to provide a first signal in a first detection channel, and a second label associated with a primer and/or probe of a second target region may be configured to provide a second signal also in the first detection channel. In some embodiments, the first and second labels may be different. In some embodiments, the first and second labels are the same. In cases where the first and second labels are different, each label may provide a detectable signal having different emission spectra, both detectable within the same channel. In some other cases where the first and second labels are different, each label may provide a detectable signal having different emission spectra, each detectable within a different channel. In some cases where the first and second labels are the same, a single detection channel is shared by the first and second labels. A detection channel may share more than two target regions, optionally from the same target nucleic acid sequence of interest (e.g., a single target gene or genome). In some embodiments, a single detection channel may be used for detection of more than two target regions and/or for more than two labels. The two target regions may be from the same or different genes, or from different tissues in the same target organism, or from two different target organisms.

In some embodiments, a detection channel may be associated with a particular target gene such that all of the target regions of that target gene, when amplified, provide signals within the same detection channel. For example, each detection channel may be associated with a separate target gene. Continuing the example above, the first target region and the second target region may both be within a first target gene and may both include the same label, and thus the first target gene is associated with the first detection channel. Other target genes can be associated with separate detection channels. For example, third and fourth target regions may be within a second target gene, and a third label associated with a primer and/or probe of a third target region and a fourth label associated with a primer and/or probe of a fourth target region may be configured to respectively provide third and fourth signals in a second detection channel. The third and fourth labels, and thus third and fourth signals, will in most cases be the same, though they may be different in some instances.

Additional labels may be included, depending on the number of target regions and desired number of detection channels. For example, primers and/or probes for amplifying other target regions (e.g., fifth, sixth, seventh, eighth, etc.) may include respective labels, and those labels may be set as different from one another or as shared across two or more target regions based on desired division of detection channels. Labels utilized in the described embodiments include VIC, FAM, JUN, ABY, Alexa Fluor (e.g., AF647 and AF676) dye labels, and combinations thereof.

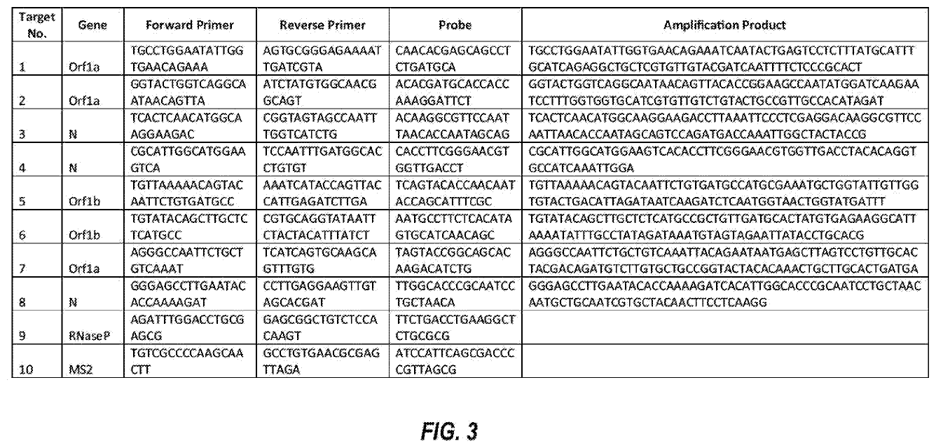

FIG. 3 illustrates a set of exemplary assays (each corresponding to a different “Target No.”) that may be used in any combination with one another to provide variant-resistant detection of SARS-CoV-2. FIG. 3 illustrates exemplary forward primers (corresponding to SEQ ID NO:1-SEQ ID NO:10), reverse primers (corresponding to SEQ ID NO:11-SEQ ID NO:20), and probes (corresponding to SEQ ID NO:21-SEQ ID NO:30) that may be utilized to detect the presence of nucleic acid target regions of SARS-CoV-2. The associated amplification products or “amplicons” (corresponding to SEQ ID NO:31-SEQ ID NO:38; amplification products for the example RNase P and MS2 controls not shown), generated if the target region is present within a sample, are also shown.

As shown in FIG. 3, the example set of assays are redundant by including multiple different target genes (Orf1a, Orf1b, and N). The example set of assays is further redundant by including multiple target regions within the target genes. In this particular example: the first, second, and seventh target regions are all associated with the Orf1a gene; the third, fourth, and eighth target regions are all associated with the N gene; and the fifth and sixth target regions are both associated with the Orf1b gene.

The illustrated set of assays does not include a target region within the S gene of SARS-CoV-2. Although other embodiments may include components that target regions within the S gene, preferred embodiments include components that target at least one, more preferably at least two, at least three, at least four, at least five, at least six, at least seven, or at least eight regions not in the S gene. The S gene is associated with several mutations that have led to “S gene dropout” of detection even when present in a sample. Thus, while still a valid location to target using the disclosed embodiments, it is more preferable to utilize other genomic targets to supplement S gene detection (e.g., M gene, E gene, and/or other gene targets described herein).

### Sample Collection

The disclosed compositions, kits, and methods are configured to detect target nucleic acid from a sample. The sample may be a veterinary sample, a clinical sample, a food sample, a forensic sample, an environmental sample (e.g., soil, dirt, garbage, sewage, air, or water), including food processing and manufacturing surfaces, or a biological sample. In some embodiments, the sample is a human sample. In some embodiments, the sample is a non-human sample. For instance, the sample may be from a non-human species such as a dog, cat, mink, etcetera. In most instances, SARS-CoV-2 or other coronaviruses and respiratory tract pathogens are detected by analysis of swabs or fluid obtained from swabs, such as throat swabs, nasal swabs (“NS”), nasopharyngeal swabs (“NP”), cheek swabs, saliva swabs, or other swabs, though it should be appreciated that SARS-CoV-2 or other coronaviruses, respiratory tract pathogens, and/or other target organisms may also be detected by analysis of urine samples, saliva samples, or other clinical samples. Such samples may be collected with a collection device such as a tube, a dish, a bag, a plate, or any other appropriate container.

The sample can be collected by a healthcare professional in a healthcare setting, but in some instances, the sample may also be collected by the patient themselves or by an individual assisting the patient in self-collection. For example, a nasopharyngeal swab has heretofore served as the gold standard for obtaining a patient sample to be used in clinical diagnostics. Such swabs are often used by a healthcare professional in a healthcare setting. Other samples, such as a saliva sample, can similarly be obtained in a healthcare setting with the assistance or oversight of a healthcare professional. However, in some instances, self-collection of a sample can be more efficient and can be done outside of a healthcare setting.

In some embodiments, the sample is a raw saliva sample collected—whether by self-collection or assisted/supervised collection—in a sterile tube or specifically-designed saliva collection device. The saliva collection tube/device may be a component of a self-collection kit having instructions for use, such as sample collection instructions, sample preparation or storage instructions, and/or shipping instructions. The raw saliva sample can be collected directly into a sealable container without any preservation solution or other fluid or substance in the container prior to receipt of the saliva sample within the container or as a result of closing/sealing the container.

Traditionally, a nucleic acid fraction of the sample is extracted or purified from the sample—whether obtained via swab, from raw saliva, or other bodily fluid—prior to any detection of viral nucleic acids therein. Surprisingly, the disclosed embodiments for detecting nucleic acid from a sample can optionally be adapted to detect viral nucleic acid directly from a raw saliva sample without a specific nucleic acid purification and/or extraction step prior to its use in downstream detection assays (e.g., RT-qPCR). In some embodiments, the saliva sample is pre-treated prior to use. This can include, for example, heating the saliva sample, such as by placing the raw saliva sample on a heat block/water bath set to a temperature of 95° C. for 30 minutes, followed by combining the heat-treated saliva with a buffer or lysis solution. The buffer or lysis solution can include, for example, any nucleic-acid-amenable buffer such as TBE and may further include a detergent and/or emulsifier such as the polysorbate-type nonionic surfactant, Tween-20.

A nucleic acid fraction of a sample obtained by a swab can optionally be extracted and used for downstream analysis, such as RT-qPCR. In some embodiments, the sample is a raw saliva sample. As provided above, the raw saliva sample can be self-collected (e.g., within a saliva collection device or sterile tube) or collected from the patient by any other individual in proximity to the patient. In some embodiments, the raw saliva sample is collected directly into a sealable container without any preservation solution or other fluid or substance in the container prior to receipt of the saliva sample or as a result of closing/sealing the container. The disclosed embodiments for detecting viral nucleic acid from a sample can be adapted to detect viral nucleic acid directly from the saliva sample, or in alternative embodiments, the sample can undergo a specific RNA purification and/or extraction step prior to its use in a detection assay (e.g., RT-qPCR). Thus, it should be appreciated that in some embodiments, a patient sample (e.g., saliva) can directly serve as sample input for subsequent downstream analyses, such as PCR, and this can be accomplished, in some embodiments, with no nucleic acid purification and/or extraction step prior to its use. In some embodiments, the sample used in subsequent downstream analyses is a heat-treated saliva sample as described herein.

In some implementations, viral nucleic acid may be detected directly from a raw saliva sample. For example, a raw saliva sample can be collected from the patient and heat-treated, such as by placing the raw saliva sample on a heat block/water bath set to a temperature of about 95° C. for 30 minutes. The heating step can provide many benefits, including, for example, denaturing nucleases such as RNase within the saliva that may interfere with accurate assessments of viral presence. Heating the raw saliva sample can also break down the mucus, making the sample more amenable to manipulation with laboratory equipment such as pipettes. The high heat can also cause thermal disruption of any prokaryotic and eukaryotic cells present in the sample and can also disrupt enveloped viruses and/or viral capsids present in the sample and thereby increase accessibility to any viral nucleic acid.

The heat-treated sample may also be mixed (e.g., via vortexing the sample for at least 10 seconds) before and/or after equilibrating the heat-treated sample to room temperature. A lysis solution can then be prepared and combined (e.g., in 1:1 proportions) with the heat-treated sample to create a probative template solution for detecting the presence of viral nucleic acid within the sample via nucleic acid amplification reactions (e.g., PCR, RT-PCR, qPCR, RT-qPCR, or the like). The lysis solution can include a nucleic-acid-amenable buffer such as TBE (and/or suitable alternative known in the art) combined with a detergent and/or emulsifier such as Tween-20, the polysorbate-type nonionic surfactant (and/or suitable alternative known in the art). The detergent and/or emulsifier can promote better mixing of the reagents and may also act to increase accessibility to any viral nucleic acid within the sample (e.g., by removing lipid envelopes from virions).

It should be appreciated that in some embodiments, the disclosed compositions can include the sample mixed with a buffer and detergent/emulsifier. The sample can be added to a buffer/detergent mixture or vice versa. In some embodiments, the sample is combined with a buffer and then detergent is added to the saliva/buffer mixture. In other embodiments, the sample is directly combined with a buffer/detergent mixture. As a non-limiting example, a set of patient samples can be prepared as compositions for downstream analysis and detection of viral sequence by adding a volume of heat-treated sample for each patient into one (or a plurality) of wells in a multi-well plate. A volume of a buffer/detergent mixture (e.g., TBE+Tween-20) can then be added to each well containing a patient sample. Alternatively, a multi-well plate can be loaded with a volume of a buffer/detergent mixture into which a volume of heat-treated saliva is added. Once combined, this probative template solution can be used immediately or stored for later analysis. Such probative template solutions can also be combined with PCR reagents (e.g., buffers, dNTPs, master mixes, etc.) prior to or after storage.

In some embodiments, a sample is obtained from multiple organisms (e.g., a plurality of individuals or patients) and the multiples samples are pooled together to make a single pooled sample for testing. In some embodiments, a sample may be obtained from at least two different organisms or individuals for pooling together to form a single sample for testing. In some embodiments, a sample may be obtained from between 2 to 10 different organisms or individuals for pooling together to form a single sample for testing. In some embodiments, a sample may be obtained from 2, 3, 4, 5, 6, 7, 8, 9, or 10 different organisms or individuals for pooling together to form a single sample for testing. In some embodiments, a sample may be obtained from up to and including 5 different organisms or individuals for pooling together to form a single sample for testing. For example, a sample used for testing, according to the methods and compositions described herein, may comprise a multiplicity of samples obtained from different organisms or individuals (e.g., 2, 3, 4, 5 different individuals) which are combined together to form single samples used for subsequent detection of a pathogen such as SARS-CoV-2.

### Treatment Buffer Formulations & Direct Detection Using a Crude Biological Sample

Some embodiments include a treatment buffer for enabling direct detection of nucleic acid from a crude biological sample. In one embodiment, the treatment buffer includes a surfactant, a protease component, a chelating agent, and a buffering salt. The treatment buffer may also optionally include a saccharide, preferably a disaccharide such as sucrose, trehalose, or combination thereof. When a saccharide is included, it is typically most effective when included at a concentration of about 200 mM to about 600 mM.

The surfactant may include a nonionic detergent, a cationic detergent, a zwitterionic detergent, anionic detergent, or any combination thereof (though anionic detergents are typically less preferred due to their tendency to interfere with downstream PCR). Non-limiting examples of suitable nonionic detergents include nonyl phenoxypolyethoxylethanol (NP-40), secondary alcohol ethoxylates such as TERGITOL 15-S-9 or TERGITOL 15-S-40 (TERGITOL 15-S-9 being more preferred), Triton X-100 (i.e., 2-[4-(2,4,4-trimethylpentan-2-yl)phenoxy]ethanol), and TWEEN 20 (generically named polysorbate 20). Non-limiting examples of suitable cationic detergents include benzalkonium chloride (BZK) and didodecyldimethylammonium bromide (DDAB). Non-limiting examples of suitable zwitterionic detergents include lauryldimethylamine oxide (i.e., LDAO, DDAO), N-(Alkyl C₁₀-C₁₆)—N,N-dimethylglycine betaine (sold under trade name EMPIGEN BB), n-Tetradecyl-N,N-dimethyl-3-ammonio-1-propanesulfonate (sold under trade name ZWITTERGENT 3-14), CHAPS (i.e., 3-[(3-cholamidopropyl)dimethylammonio]-1-propanesulfonate), or CHAPSO (i.e., 3-[(3-cholamidopropyl)dimethylammonio]-2-hydroxy-1-propanesulfonate). More preferred zwitterionic detergents include LDAO, EMPIGEN BB, and ZWITTERGENT 3-14. Combinations of any of the foregoing may also be utilized. As shown in detail in the Examples below, surfactants which have proven to be particularly effective in subsequent detection of target nucleic acid (e.g., from SARS-CoV-2), include LDAO and BZK.

The surfactant may be included at a concentration of about 0.01% to about 0.10% w/v, or more preferably about 0.02% to about 0.08% w/v. Another way to determine an appropriate surfactant concentration is to include the surfactant at a concentration within 0.5× and 15× of the surfactant's critical micelle concentration (CMC). One of skill in the art will understand how to conduct a CMC test and/or consult appropriate literature to find such values for a selected surfactant.

The protease component may include a serine protease such as proteinase K. In some embodiments, the protease component includes a mixture of two or more proteases. The protease mixture may comprise a mixture of proteases isolated from a bacterial culture. One example of such a mixture is pronase, which is a mixture of proteases isolated from extracellular fluid of the actinobacteria Streptomyces griseus. As shown in detail in the Examples below, pronase has proven to be particularly effective in increasing the accessibility to viral nucleic acids in crude samples. Other proteases, including other proteases from other types of microbial cultures, may additionally or alternatively be utilized in the protease component. The protease component may be included at a concentration of about 20 U/ml to about 100 U/ml, or more preferably about 35 U/ml to about 85 U/ml, or even more preferably about 50 U/ml to about 70 U/ml.

The surfactant, protease component, or both function to inactivate virions (and/or other infectious agents or microorganisms) within the sample. The inactivation effects of the surfactant and protease component were surprisingly found to be enhanced when utilized in combination than when used independently, other conditions being equal (see Example 5 below). The treatment solution also functions to disrupt viral envelopes, cell membranes, or proteins within the crude biological sample. The treatment solution beneficially provides increased access to the target nucleic acid when mixed with the crude biological sample as compared to a mixture of the crude biological sample omitting one or more components of the treatment solution (e.g., as compared to a mixture of the sample with water and/or buffer only).

The buffering salt may include any salt or salt mixture that provides sufficient buffering functionality. Suitable salts include sodium salts (e.g., sodium citrate) and/or chloride salts (e.g., Tris-HCl). The salt concentration is preferably less than about 50 mM, such as within a range with a lower endpoint of about 2 mM and an upper endpoint of about 40 mM, 30 mM, 20 mM, or 15 mM. The chelating agent may include ethylenediaminetetraacetic acid (EDTA) or a conjugate base or salt thereof, for example. The chelating agent may be included at a concentration of about 0.3 mM to about 1.2 mM, or more preferably about 0.5 mM to about 1.0 mM.

The treatment solution may additionally include an antifoam agent, which is particularly beneficial for crude samples such as saliva that tend to foam on occasion. The antifoaming agent is preferably included in an amount of about 0.001% to about 0.008% w/v, or more preferably about 0.0015% to about 0.004% w/v. The antifoaming agent may be formulated with silicon and nonionic emulsifiers, such as the antifoam agent SE-15.

As mentioned above, the treatment solution is formulated for mixing directly with a crude biological sample. The composition can be formulated for mixing with the crude biological sample at a ratio of about 0.5:1 to about 4:1, or at a ratio of about 1:1 to about 2:1, with component amounts of the treatment composition being scaled accordingly for other mixture ratios. In other words, the concentrations of the components of the treatment composition described herein assume a mixing ratio within the foregoing ranges, but where other mixing ratios are utilized, the concentrations may be scaled accordingly.

The mixing ratio may also depend on the collection method of the sample. For example, where the treatment solution is mixed directly with a liquid sample (e.g., saliva, blood, urine, etc.), it will typically be mixed at a ratio closer to about 1:1 (e.g., 0.5:1 to 2:1), whereas when the treatment solution is mixed with a swab (or similar collection device) to resuspend material collected on the swab, the ratio will typically be higher, such as about 2:1 (e.g., 1.5:1 to 4:1).

The treatment solution is preferably formulated such that the pH is at about 7 or greater, such as about 7.2 to about 8. The treatment solution is formulated to promote stability of the solution-sample mixture. For example, mixtures can remain stable at room temperature for at least about 96 hours. The term “stable”, as used in this context, means that the solution-sample mixture may be subsequently processed with no or negligible (e.g., less than 10%) loss of sensitivity to nucleic acid detection as compared to otherwise similar solution-sample mixtures that are processed without such a waiting period.

The treatment solutions described herein beneficially provide one or more of: (i) stabilization of the crude biological sample when mixed; (ii) inactivation of at least one virus and/or microorganism within the crude biological sample; (iii) lysis of animal cells and/or the at least one virus and/or microorganism within the crude biological sample; (iv) reduction in viscosity of the crude biological sample; (v) improving accessibility of viral and/or other microorganism nucleic acids within the crude biological sample; and (vi) preserving integrity of nucleic acids within the crude biological sample without extraction or purification of the RNA. Treatment solution formulations can more beneficially provide two or more, or three or more, or even all of the foregoing functions.

Treatment solutions such as those described above can be used to process a crude biological sample containing or suspected of containing a target nucleic acid. In one embodiment, a method generally includes the steps of: (a) contacting the biological sample with a treatment solution comprising a protease component to form a mixture; (b) inactivating the protease component in the mixture of (a); and (c) performing an analysis of the target nucleic acid.

The mixing of step (a) may include any of the treatment solutions described in the above section. As mentioned above, the treatment solution may be mixed with the crude biological sample at a ratio of about 0.5:1 to about 4:1, or at a ratio of about 1:1 to about 2:1, with component amounts of the treatment composition being scaled accordingly for other mixture ratios. The biological sample thus typically makes up about 10% to 60% of the volume of the mixture of step (a). Following formation of the mixture of step (a), but prior to step (b), the mixture may be stored and/or shipped for a period of time. This period may have a duration of up to about 96 hours (assuming room temperature conditions or similar), and the mixture beneficially remains stable throughout this period.

The biological sample may include one or more of a saliva sample, buccal sample, nasal sample, nasal pharyngeal sample, blood sample, urine sample, or semen sample. The biological sample may be mixed directly with the treatment solution, or may be a resuspension of sample previously obtained using a swab or other sample collection device. The volume of such a resuspension may depend on the type of sample and particular application protocols, but is preferably a small resuspension volume of about 0.1 ml to about 1 ml.

Inactivation of the protease component in step (b) may involve temperature treatment, the addition of a protease inhibitor component, or both. The temperature treatment may include sequentially treating the mixture at a first temperature and then a second, different temperature. The first and second temperatures preferably differ by at least about 15° C. The first temperature may include a temperature between 20° to 70° C. and the second temperature may include a higher temperature. The second temperature may be varied according to the first. For example, the second temperature may typically lie between about 85° to 100° C., but may be set at a lower temperature when the first temperature is high enough to compensate. The first temperature may be room temperature, or about 25° C., for example.

The duration of incubation at the first temperature may depend on the first temperature, with the duration being longer for relatively lower temperatures and shorter for relatively higher temperatures. In other words, the higher the first temperature is, the less time needed before moving to the second temperature. The duration of incubation at the first and second temperatures may be about 2 minutes each. Incubation at the second temperature is preferably no longer than about 15 minutes.

The temperature treatment may further include incubating the mixture at a third temperature for a third time interval of time prior to performing step (c). The third temperature may be between 2° to 8° C. The third temperature may be for at least one minute, though the mixture may be stored for up to 24 hours at the third temperature prior to performing step (c).

The temperature treatment may further include incubating the mixture at a fourth temperature prior to step (c). The first and fourth temperatures may be substantially the same. For example, the fourth temperature may be about 25° C. or room temperature. The mixture is beneficially stable at the fourth temperature for at least 96 hours.

In embodiments where a protease inhibitor is added to the mixture in step (b), the protease inhibitor may include a mixture with a plurality of protease inhibitors, also referred to herein as a “protease inhibitor cocktail”. A preferred protease inhibitor cocktail is sold under the name HALT, and includes six different inhibitors: AEBSF (1 mM), aprotinin (800 nM), bestatin (50 μM), E64 (15 μM), leupeptin (20 μM), and pepstatin A (10 μM). One or more of such inhibitors may be utilized independently or in any combination, with preferred embodiments using multiple inhibitors.

The analysis of step (c) may include amplifying one or more target nucleic acids within the biological sample. The described methods beneficially enable more efficient amplification of the target nucleic acid, resulting in a lower Ct value, as compared to otherwise similar samples in water and/or TE buffer. In some embodiments, multiple different nucleic acids are amplified, such as in a multiplex reaction. For example, a first target nucleic acid may be from a target virus or microbe, while a second target nucleic acid is from the organism from which the biological sample is obtained (e.g., from a patient). The second target nucleic acid may be an RNase P nucleic acid, for example. In other embodiments, a first target nucleic acid may be from a target virus or microbe, while a second target nucleic acid is an external positive control nucleic acid, such as bacteriophage MS2 control nucleic acid.

Steps (a) through (c) may be performed in a single reaction vessel or multiple reaction vessels. For example, steps (a) and (b) may be performed in a first reaction vessel or tube and step (c) is performed in a second reaction vessel or tube. In some embodiments, an aliquot of the mixture from (b) is transferred to the second reaction vessel and further diluted prior to performing step (c). The aliquot of the mixture from (b) may be mixed with one or more PCR reagents in the second reaction vessel.

### Nucleic Acid Amplification & Detection

The amplified products (“amplicons”) resulting from use of the component of the assays described herein can be detected and/or analyzed using any suitable method and on any suitable platform. In some embodiments, SARS-CoV-2 or other target organisms are detected by analysis of swabs, or fluid obtained from swabs, such as throat swabs, nasal swabs, nasopharyngeal swabs, cheek swabs, saliva swabs, or other swabs. SARS-CoV-2 or other target organisms may also be detected by analysis of saliva samples, buccal samples, nasal samples, nasal pharyngeal samples, blood samples, urine samples, semen samples, or other biological samples.

In some embodiments, the nucleic acid assays as described herein can be used to detect, identify, characterize, quantify, or otherwise measure one or more nucleic acid targets in a sample. In some embodiments, the nucleic acid targets may be single stranded, double stranded, or any other nucleic acid molecule of any size. Optionally, the nucleic acid assays described herein can include polymerase chain reaction (PCR) assays (see, e.g., U.S. Pat. No. 4,683,202), loop-mediated isothermal amplification (“LAMP”) (see, e.g., U.S. Pat. No. 6,410,278), and other methods, including methods discussed below for detecting nucleic acid targets in a sample. In some embodiments, the PCR assays can be real time PCR or quantitative (qPCR) assays. In some other embodiments, the PCR assays can be end point PCR assays. Nucleic acid markers may be detected by any suitable means, including means that include nucleic acid amplification (e.g., thermal cycling amplification methods including PCR, and other nucleic acid amplification methods; isothermal amplification methods, including LAMP, etc.) and any other method that can be used to detect the presence of nucleic acid markers indicative of a disease-causing organism in a sample.

In some embodiments, the primers described herein are used in nucleic acid assays at a concentration from about 100 nM to 1 mM (e.g., 300 nM, 400 nM, 500 nM, etc.), including all concentration amounts and ranges in between. In some embodiments, the probes described herein are used in a nucleic acid assay at a concentration from about 50 nM to 500 nM (e.g., 75 nM, 125 nM, 250 nM, etc.), including all concentration amounts and ranges in between.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels include but are not limited to 6FAM, ABY, VIC, JUN, FAM. Suitable quenchers include but are not limited to QSY (e.g., QSY7 and QSY21), BHQ (Black Hole Quencher) and DFQ (Dark Fluorescent Quencher). Optionally, in some embodiments, control sequence primers and/or probes (e.g., JUN-labeled probes), such as for amplification and/or detection of bacteriophage MS2 or human RNase P control sequences, are included in the multiplex assays using primer/probe sequences disclosed herein (and may be included as singleplex assays as well).

In some embodiments the described assays can be run as singleplex assays or as a multiplex assay. In some embodiments, the panel includes some combination of one or more assays present in the TaqMan™ Array Respiratory Tract Microbiota Comprehensive Card (Thermo Fisher Scientific, Waltham, MA; Catalog No. A41238), along with one or more assays described herein (e.g., as shown in FIG. 3) in at least one well of the array. In some embodiments, the panel includes assays for other circulating coronavirus strains, including but not limited to 229E, KHU1, NL63, and OC43. In some embodiments, the disclosed methods include using the panel to profile respiratory microorganisms present in a sample taken from an organism (e.g., human) and determining the profile of respiratory microbiota present in the organism's sample. Optionally, the disclosed methods can include diagnosing an infection present in an organism (e.g., human) from which a sample is taken.

In some embodiments, a panel of different qPCR assays can be used to test for multiple strains or types of pathogens in addition to SARS-CoV-2, and variants thereof, including, but not limited to, other viral pathogens such as Influenza Type A and/or Type B, and RSV Type A and/or Type B, bacterial pathogens, and/or fungal pathogens. In some embodiments, the panel of qPCR assays can be used simultaneously to test a single patient sample or a single pooled sample comprising multiple patient samples, with each assay run in parallel in array format (“array formatted”). Optionally, different qPCR assays specific for each of the following target assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use.

In some embodiments, the array of qPCR assays includes at least one qPCR assay for detecting SARS-CoV-2, plus at least one qPCR assay for detecting one or more of respiratory microorganisms listed in Table 5, below. Each qPCR assay can include a forward primer and a reverse primer for each target. Optionally, the assay can further include one or more probes.

TABLE 5
Respiratory Microorganisms

| | | | | |
|---|---|---|---|---|
| AdV 1 of 2 | FluB | CoV HKU1 | ParechoV | M. catarrhalis |
| AdV 2 of 2 | (pan) | CoV NL63 | SARS-CoV-2-1 | M. pneumoniae |
| HBoV | hPIV1 | CoV OC43 | Bordetella spp. | S. aureus |
| HHV3 | hPIV2 | Mumps | B. holmesii | S. pneumoniae |
| HHV4 | hPIV3 | MERS-CoV | B. pertussis | P. jirovecii |
| HHV5 | hPIV4 | SARS-CoV | SARS-CoV-2-2 | SARS-CoV-2-3 |
| HHV6 | RSVA | EnteroV | C. pneumoniae | Internal |
| FluA (pan) | RSVB | (pan) | H. influenzae | Control |
| FluA (H1) | hMPV | EnteroV D68 | K. pneumoniae | (IC) - RNase P |
| FluA (H3) | Measles | RhinoV 1of2 | L. pneumophila | |
| | CoV 229E | RhinoV 2of2 | | |

In some embodiments, the multiplex assay detects two or more (e.g., 2, 3, 4, 5, 6, etc.) of the targets of Table 5. In some embodiments, the multiplex assay detects one or more targets within the SARS-CoV-2 genome (e.g., including reference and/or mutant or variant SARS-CoV-2 targets) as wells an internal positive control, such as RNase P.

In some embodiments, the primers and/or probes described herein can be used to amplify one or more specific target sequences present in a SARS-CoV-2 target and to enable robust, variant-resistant identification of SARS-CoV-2. In some embodiments, the primers and/or probes described herein can accurately detect known and future SARS-CoV-2 variants, including the B.1.1.7 variant (“UK variant”) and/or the B.1.351 variant (“S. African variant”), for example.

The primer and probe sequences described herein need not have 100% homology to their targets to be effective, though in some embodiments, homology is substantially 100%. In some embodiments, one or more of the disclosed primer and/or probe sequences have a homology to their respective target of at least about 50%, at least about 60%, at least about 70%, at least about 80%, at least about 85%, at least about 90%, at least about 95%, at least about 97%, at least about 98%, at least about 99%, at least about 99.9%, or up to 100%. Some combinations of primers and/or probes may include primers and/or probes each with different homologies to their respective targets, and the homologies may be, for example, within a range with endpoints defined by any two of the foregoing values.

Polymerase chain reaction (PCR) and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as, e.g., loop-mediated isothermal amplification (“LAMP”), and other isothermal methods, such as those listed in Table 6, may require less or less extensive thermal cycling than does PCR, or require no thermal cycling. Such isothermal amplification methods are also contemplated for use with the compositions, kits, and methods described herein.

TABLE 6
Summary of optional isothermal amplification methods.

| | |
|---|---|
| NASBA | Nucleic acid sequence-based amplification (NASBA) is a method |
| | used to amplify RNA. |
| LAMP | Loop-mediated isothermal amplification (LAMP) is a single tube |
| | technique for the amplification of DNA. It typically uses 4-6 primers, which |
| | form loop structures to facilitate subsequent rounds of amplification. |
| HDA | Helicase-dependent amplification (HDA) uses the double-stranded |
| | DNA unwinding activity of a helicase to separate strands for in vitro DNA |
| | amplification at constant temperature. |
| RCA | Rolling circle amplification (RCA) starts from a circular DNA |
| | template and a short DNA or RNA primer to form a long single stranded |
| | molecule. |
| MDA | Multiple displacement amplification (MDA) is a technique that |
| | initiates when multiple random primers anneal to the DNA template and the |
| | polymerase amplifies DNA at constant temperature. |
| WGA | When MDA is used to amplify DNA from a whole genome of a cell it |
| | is called whole genome amplification (WGA). (Other methods of WGA |
| | include MALBAC, LIANTI, DOP-PCR.) |
| RPA | Recombinase polymerase amplification (RPA) is a low temperature |
| | DNA and RNA amplification technique. |

Methods of performing PCR are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

SARS-CoV-2 has a single-stranded positive-sense RNA genome. In some embodiments, therefore, the amplification reaction (e.g., LAMP or PCR) can be combined with a reverse transcription (RT) reaction, such as in RT-LAMP or RT-PCR to convert the RNA genome to a cDNA template. The cDNA template is then used to create amplicons of the target sequences in the subsequent amplification reactions. In some embodiments, RT-PCR is performed using samples comprising virus particles or suspected of comprising virus particles. In some embodiments, the viral particles are live particles. In some embodiments, the viral particles are dead or inactivated particles. In some embodiments, the RT-PCR may be a one-step procedure using one or more primers and one or more probes as described herein.

In some embodiments, the RT-PCR may be carried out in a single reaction tube, reaction vessel (e.g., “single-tube” or “1-tube” or “single-vessel” reaction). In some embodiments, the RT-PCR may be carried out in a multi-site reaction vessel, such as a multi-well plate or array. In some embodiments, RT and PCR are performed in the same reaction vessel or reaction site, such as in 1-step or 1-tube RT-qPCR. Suitable exemplary RTs can include, for instance, a Moloney Murine Leukemia Virus (M-MLV) Reverse transcriptase, SuperScript Reverse Transcriptases (Thermo Fisher Scientific), SuperScript IV Reverse Transcriptases (Thermo Fisher Scientific), or Maxima Reverse Transcriptases (Thermo Fisher Scientific), or modified forms of any such RTs.

In some embodiments, different assay products (e.g., amplicons from different variants) can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety.

In some embodiments, the amplifying step can include performing qPCR, as that term is defined herein. qPCR is a sensitive and specific method for detecting and optionally quantifying amounts of starting nucleic acid template (e.g., coronaviral nucleic acid) in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Suitable labels are described herein.

The reaction vessel or volume can optionally include a tube, channel, well, cavity, site or feature on a surface, or alternatively a droplet (e.g., a microdroplet or nanodroplet) that may be deposited onto a surface or into a surface well or cavity, or suspended within (or partially bounded by) a fluid stream. In some embodiments, the reaction volume includes one or more droplets arrayed on a surface or present in an emulsion. The reaction volumes can optionally be formed by fusion of multiple pre-reaction volumes containing different components of an amplification reaction. For example, pre-reaction volumes containing one or more primers can be fused with pre-reaction volumes containing human nucleic acid samples and/or polymerase enzymes, nucleotides, and buffer. In some embodiments involving performing qPCR reactions in array format, a surface contains multiple grooves, channels, wells, cavities, sites, or features defining a reaction volume containing one or more amplification reagents (e.g., primers, probes, buffer, polymerase, nucleotides, and the like). In some array-formatted singleplex embodiments, the reaction volume within the selected tubes, grooves, channels, wells, cavities, sites, or features contains only a single forward primer sequence and a single reverse primer sequence. Optionally, one or more probe sequences are also included in the singleplex reaction volume.

In some array-formatted multiplex embodiments, the reaction volume within the selected tubes, grooves, channels, wells, cavities, sites, or features contains multiple (e.g., 2, 3, 4, 5, 6, etc.) forward and reverse primer sequences and/or multiple probe sequences. For instance, exemplary methods for polymerizing and/or amplifying and detecting nucleic acids suitable for use as described herein are commercially available as TaqMan assays (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety).

TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. The oligonucleotide probe typically includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

For example, a singleplex or multiplex qPCR can include a single TaqMan assay associated with a locus-specific sequence or multiple TaqMan assays respectively associated with a plurality of loci in a multiplex format. As a non-limiting example, a 4-plex reaction can include FAM (emission peak ˜517 nm), VIC (emission peak ˜551 nm), ABY (emission peak ˜580 nm), and JUN (emission peak ˜617 nm) dyes. In some embodiments, each dye is associated with a different target sequence. In some embodiments, each dye is associated with two or more target sequences. In some embodiments, one or more dyes are quenched by a QSY quencher (e.g., QSY21). In some embodiments, each multiplex reaction allows up to 12 targets (e.g., 2, 4, 6, 8, 10, or 12 targets) to be amplified and tracked real-time within a single reaction vessel. In some embodiments, up to 2, 4, 6, 8, 9, 10, or 12 targets are amplified and tracked real-time within a single reaction vessel, using any combination of detectable labels disclosed herein or otherwise known to those of skill in the art. The aforementioned reporter dyes are optimized to work together with minimal spectral overlap for improved performance. Any combination of dyes described herein can additionally be combined with other dyes (e.g., Mustang Purple (emission peak ˜654 nm) or one or more Alexa Fluors (e.g., AF647 and AF676)), for use in monitoring fluorescence of a control or for use in a non-emission-spectrum-overlapping 5-plex assay. In addition, the QSY quencher is fully compatible with probes that have minor-groove binder quenchers.

In some embodiments, an assay may be multiplex in the sense that it is configured for detecting the presence of multiple target nucleic acid regions within the sample. An assay may additionally be multiplex in the sense that it is configured to use multiple detection channels for the detection of the multiple target regions. The number of target regions analyzed by the assay may be different from the number of detection channels utilized by the assay. In other words, the “plexy” of an assay with respect to target nucleic acid regions may be different than the “plexy” of the assay with respect to detection channels. For example, the set of assays shown in FIG. 3 can be combined to form a combined assay that is 9-plex with respect to target regions analyzed (e.g., including each “Target No.” 1-8 and one of the control targets selected from “Target No.” 9 or 10), but is 4-plex with respect to the number of detection channels utilized (e.g., using FAM for the three Orf1a target regions, ABY for the two Orf1b target regions, VIC for the three N gene target regions, and JUN for the positive control). Other combinations are also possible. For example, an assay may be 1-, 2-, 3-, 4-, 5-, 6-, 7-, 8-, 9-, 10-, 11-, or 12-plex (or higher) with respect to the number of target regions analyzed, and be 1-, 2-, 3-, 4-, 5-, 6-, 7-, 8-, 9-, 10-, 11-, or 12-plex (or higher) with respect to the number of detection channels utilized. As discussed above, multiple target regions may be associated with the same target gene and/or same target organism, but in at least some embodiments one or more target regions may be separately associated with different target genes or different target organisms.

Where multiple detection channels are utilized, it is desirable to minimize cross-talk between fluorescence reporters and select reporters that avoid excessive spectral overlap. One example of an assay that is 5-plex with respect to detection channels incorporates the dyes FAM, ABY, VIC, and JUN, along with Mustang Purple (emission peak ˜654 nm) or an appropriate Alexa Fluor, for example. The dyes may be associated with a corresponding primer and/or with a probe of the assay, as described herein. Other embodiments may utilize other combinations of dyes to define different sets of detection channels (including in assays with more than 5 detection channels) according to particular preferences or application needs. Additional examples of multiplex assays (including related dye compounds, compositions, methods, and kits) are described in U.S. Provisional Patent Application No. 62/705,935, filed Jul. 23, 2020 and titled “Compositions, Systems and Methods for Biological Analysis Involving Energy Transfer Dye Conjugates and Analytes Comprising the Same”, which is incorporated herein by this reference in its entirety.

Detector probes may be associated with alternative quenchers, including without limitation, dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy5.

It should be appreciated that when using more than one detectable label, particularly in a multiplex format, each detectable label preferably differs in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′,7′-dimethoxy¬fluorescein (JOE)); 6-carboxy-1,4-dichloro-2′,7′-dichloro¬fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalama1), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl, rhodamine), Tetramethylrhodamine (TRITC), WT), Texas Red, Texas Red-X, among others as would be known to those of skill in the art.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30(9): e37), Hayashi et al. (Nucleic Acids Res. 1989 May 11; 17(9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

In some embodiments, the amplicon is labeled by incorporation of or hybridization to labeled primer. In some embodiments, the amplicon is labeled by hybridization to a labeled probe. In some embodiments, the amplicon is labeled by binding of a DNA-binding dye. In some embodiments, the dye may be a single-strand DNA binding dye. In other embodiments, the dye may be a double-stranded DNA binding dye. In other embodiments, the amplicon is labeled via polymerization or incorporation of labeled nucleotides in a template-dependent (or template-independent) polymerization reaction. This can be part of the amplifying step or alternatively the labeled nucleotide can be added after amplifying is completed. The labeled amplicon (or labeled derivative thereof) can be detected using any suitable method such as, for example, electrophoresis, hybridization-based detection (e.g., microarray, molecular beacons, and the like), chromatography, NMR, and the like.

In one exemplary embodiment, the labeled amplicon is detected using capillary electrophoresis. In another embodiment, the labeled amplicon is detected using qPCR. In some embodiments, a plurality of different amplicons are formed, and optionally labeled, within a single reaction volume via a single amplification reaction. For example, a multiplex reaction (e.g., 2-plex, 3-plex, 4-plex, 5-plex, 6-plex) carried out in a single tube or reaction vessel (e.g., “single-tube” or “1-tube” or “single-vessel” reaction) can produce a plurality of amplicons that are labeled. In some embodiments, the plurality of amplicons can be differentially labeled. In some embodiments, each of the plurality of amplicons produced during amplification is labeled with a different label.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), QuantStudio 7 RealTime PCR System (QS7), and/or QuantStudio 12K Flex System (QS12K), or a 7500 Real-Time PCR system, such as the 7500 Fast Dx system, from Thermo Fisher Scientific.

In some embodiments, the systems, compositions, methods, and devices used for nucleic acid amplification comprise a “point-of-service” (POS) system. In some embodiments, samples may be collected and/or analyzed at a “point-of-care” (POC) location. In some embodiments, analysis at a POC location typically does not require specialized equipment and has rapid and easy-to-read visual results. In some embodiments, analysis can be performed in the field, in a home setting, and/or by a lay person not having specialized skills. In certain embodiments, for example, the analysis of a small-volume clinical sample may be completed using a POS system in a short period of time (e.g., within hours or minutes).

Optionally, a POS system is utilized at a location that is capable of providing a service (e.g., testing, monitoring, treatment, diagnosis, guidance, sample collection, verification of identity (ID verification), and other services) at or near the site or location of the subject. A service may be a medical service or it may be a non-medical service. In some situations, a POS system provides a service at a predetermined location, such as a subject's home, school, or work, or at a grocery store, a drug store, a community center, a clinic, a doctor's office, a hospital, an outdoor triage tent, a makeshift hospital, a border check point, etc. A POS system can include one or more point of service devices, such as a portable virus/pathogen detector. In some embodiments, a POS system is a point of care system. In some embodiments, the POS system is suitable for use by non-specialized workers or personnel, such as nurses, police officers, civilian volunteers, or the patient.

In certain embodiments, a POC system is utilized at a location at which medical-related care (e.g., treatment, testing, monitoring, diagnosis, counseling, etc.) is provided. A POC may be, e.g., at a subject's home, work, or school, or at a grocery store, a community center, a drug store, a doctor's office, a clinic, a hospital, an outdoor triage tent, a makeshift hospital, a border check point, etc. A POC system is a system which may aid in, or may be used in, providing such medical-related care, and may be located at or near the site or location of the subject or the subject's health care provider (e.g., subject's home, work, or school, or at a grocery store, a community center, a drug store, a doctor's office, a clinic, a hospital, etc.).

In embodiments, a POS system is configured to accept a clinical sample obtained from a subject at the associated POS location. In embodiments, a POS system is further configured to analyze the clinical sample at the POS location. In embodiments, the clinical sample is a small volume clinical sample. In embodiments, the clinical sample is analyzed in a short period of time. In embodiments, the short period of time is determined with respect to the time at which sample analysis began. In embodiments, the short period of time is determined with respect to the time at which the sample was inserted into a device for the analysis of the sample. In embodiments, the short period of time is determined with respect to the time at which the sample was obtained from the subject.

In some embodiments, a POS system or a POC system can include the amplification-based methods, compositions and kits disclosed herein, including any of the described assays and/or assay panels. Such assays are contemplated for use with both thermal cycling amplification workflows and protocols, such as in PCR, as well as isothermal amplification workflows and protocols, such as in LAMP.

In some embodiments, a POS or a POC system comprises self-collection of a biological sample, such as a nasal swab or a saliva sample. In some embodiments, the self-collection may comprise the use of a self-collection kit and/or device, such as a swab or a tube (e.g., a saliva collection tube or similar saliva collection device). In some embodiments, the self-collection kit comprises instructions for use, including collection instructions, sample preparation or storage instructions, and/or shipping instructions. For example, the self-collection kit and/or device may be used by an individual, such as lay person, not having specialized skills or medical expertise. In some embodiments, self-collection may be performed by the patient themselves or by any other individual in proximity to the patient, such as but not limited to a parent, a care giver, a teacher, a friend, or other family member.

Notably, in some embodiments, the nucleic acid amplification protocol can be configured for rapid processing (e.g., in less than about 45 minutes) and high-throughput, allowing for a minimally invasive method to quickly screen large numbers of individuals in a scalable way. This can be particularly useful to perform asymptomatic testing (e.g., high frequency/widespread testing at schools, workplaces, conventions, sporting events, large social gatherings, etc.) or for epidemiological purposes. The disclosed embodiments can also beneficially provide a lower cost sample collection system and method that enables self-collection (reducing health care professional staffing needs) using a low-cost collection device. This eliminates the requirements for swabs, buffers, virus transmission media (or other specialized transport medium), and the like. The disclosed embodiments also allow for a reduction in Personal Protective Equipment (PPE) requirements and costs. Because the reagents and methods are streamlined (e.g., no precursor nucleic acid purification and/or extraction step), there is a reduced use of nucleic acid preparation plastics which brings a coincident reduction in reagent costs and inventory costs. There is also a beneficial reduced dependence on supply-constrained items, and the compatibility of these methods and kit components with existing equipment improves the flexibility and simplicity of their implementation to the masses. Overall, such embodiments allow for a less expensive assay that can be accomplished more quickly from sample collection through result generation.

Some embodiments relate to kits containing one or more of the primers and probes disclosed in FIG. 3. Optionally, the kit can further include a master mix. In some embodiments, the master mix is TaqMan Fast Virus 1-Step Master Mix (Thermo Fisher Scientific, Waltham, MA, Catalog No. 44444432). In some embodiments, the master mix is TaqPath 1-Step RT-qPCR Master Mix, CG (Thermo Fisher Scientific, Waltham, MA, Catalog No. A15299). In other embodiments the master mix is TaqPath™ 1 Step Multiplex Master Mix (No ROX™) (Thermo Fisher Scientific, Waltham, MA, Catalog No. A48111, A28521). In some embodiments, the kit includes primers, probes, and master mix sufficient to constitute a reaction mixture supporting amplification of one or more target regions from SARS-CoV-2 and/or other target organisms.

In some array-based embodiments, two or more different qPCR assays (each containing a forward primer, a reverse primer and optionally a probe) are used in a single well, cavity, site or feature of the array and products of each assay can be independently detected. For example, different assay products may be discriminated optically (e.g., using different labels present in components each assay) or using some other suitable method, including as described in U.S. Patent Publication No. 20190002963, incorporated by reference herein. In some embodiments, at least one primer of each assay contains an optically detectable label that can be discriminated from the optical label of at least one other assay.

In some embodiments, optimal amplification and detectability for viral genomes is achieved by adding a master mix to the reaction volume prior to amplification. The master mix optionally includes a polymerase, nucleotides, buffers, and salts. In some embodiments (particularly multiplex assays), the reaction volume includes TaqMan Fast Virus 1-Step Master Mix (Thermo Fisher Scientific, Waltham, MA, Catalog No. 44444432). In some embodiments, the reaction volume includes TaqPath 1-Step RT-qPCR Master Mix, CG (Thermo Fisher Scientific, Waltham, MA, Catalog No. A15299). In other embodiments the master mix is TaqPath™ 1 Step Multiplex Master Mix (No ROX™) (Thermo Fisher Scientific, Waltham, MA, Catalog No. A48111, A28521).

### Control Templates/Assays

Optionally, in some embodiments, a control template and/or assay, such as bacteriophage MS2 or RNase P control, is included in the kit. If the positive control sequence is an endogenously-derived control, such as RNase P, the presence of patient-derived nucleic acid (e.g., genomic DNA coding for RNase P, RNase P RNA, and/or reverse transcribed RNase P transcript), can be used as the template for an RNase P qPCR assay. If the positive control sequence is an exogenously-derived control, such as a component of the MS2 bacteriophage, a known or predetermined concentration of template nucleic acid can be added to the reaction volume to serve as the requisite template for an MS2 positive control qPCR assay.

In some embodiments, the methods, systems and assays of the disclosure related to use of one or more target control sequences that can be used to confirm or validate any of the steps in an assay workflow, including for example extraction, lysis or nucleic acid amplification. In some embodiments, a control template and/or assay may contain one or more nucleic acid constructs. In some embodiments, the one or more nucleic acid constructs have one or more target control sequences. In some embodiments, the one or more target control sequences include one or more sequences that contain or consist of the sequence of any amplified target sequence that is being amplified or otherwise interrogated in an assay. Optionally, the one or more target control sequences can be amplified by use of a first primer selected from selected from SEQ ID NO:1 to SEQ ID NO:10 and a second primer selected from selected from SEQ ID NO:11 to SEQ ID NO:20, or the complements thereof. In some embodiments, the one or more target control sequences have one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:10 and a second primer selected from selected from SEQ ID NO:11 to SEQ ID NO:20, or the complements thereof. In some embodiments, the first primer is a forward primer and the second primer is a reverse primer. In some other embodiments, the first primer is a reverse primer and the second primer is a forward primer. In some embodiments, each of the pairs of first and the second primers amplify sequences that are different. In some embodiments, the one or more nucleic acid constructs have additional control sequences such as bacteriophage MS2 or human RNase P control sequences.

In some embodiments, a control template and/or assay contains one nucleic acid construct that has one or more target control sequences and one or more additional control sequences (e.g., MS2 or RNase P control sequences) in a single molecule. In some other embodiments, a control template and/or assay has two or more nucleic acid constructs. In such embodiments, one nucleic acid construct has one or more target control sequences whereas the other nucleic acid construct has one or more additional control sequences. In some other embodiments, a control template and/or assay has three or more nucleic acid constructs. In such embodiments, at least two of the nucleic acid constructs have the target control sequences and the additional control sequences are provided into a separate nucleic acid construct that does not have the target control sequences.

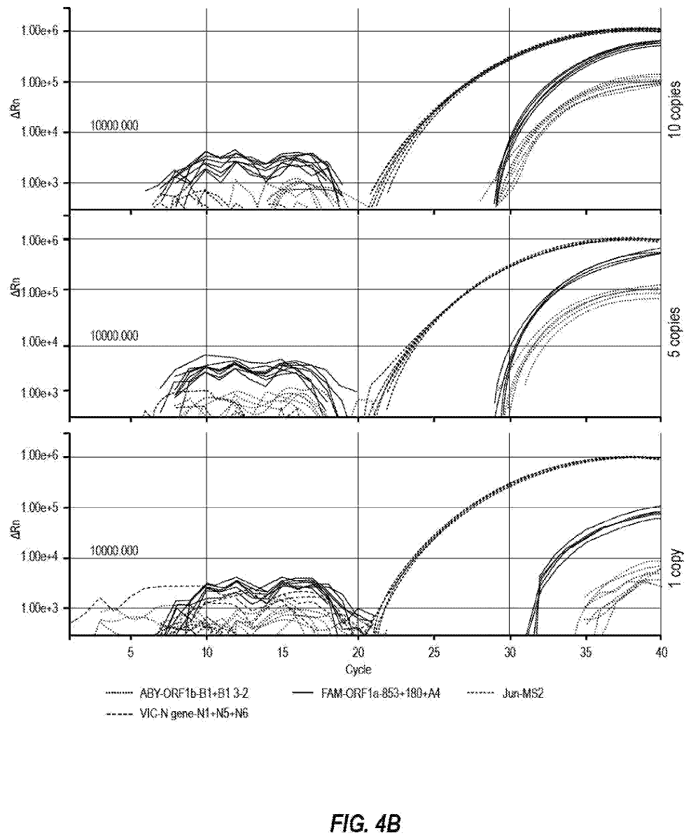

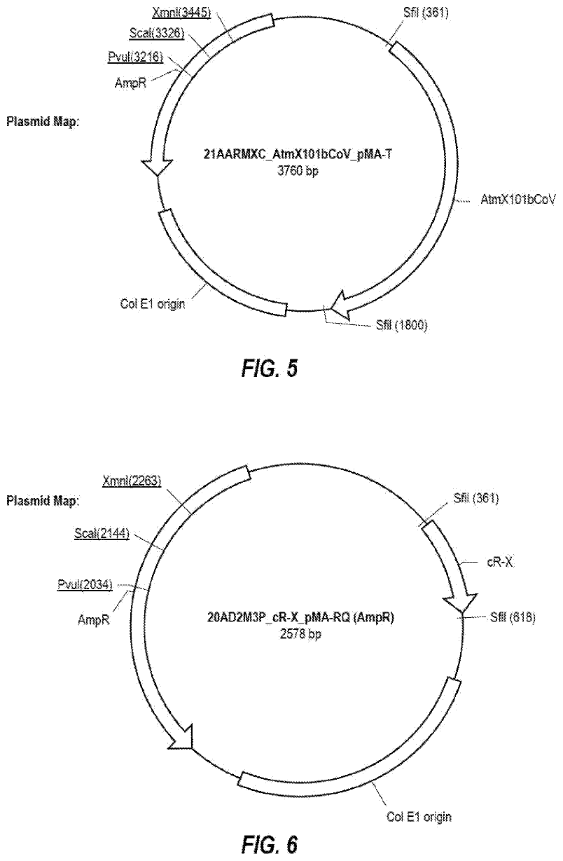

In some embodiments, one or more nucleic acid constructs from a control template and/or assay is DNA, which can be single-stranded or double-stranded. In some embodiments, one or more nucleic acid constructs from a control template and/or assay is RNA, which can be single-stranded or double-stranded. In some embodiments where two or more nucleic acid constructs are provided, the constructs can be a mixture of DNA and RNA. Thus, in some embodiments where two or more nucleic acid constructs are provided, at least one construct is DNA and at least another construct is RNA. In other embodiments, all nucleic acid constructs are either DNA or RNA. In some embodiments where at least one of nucleic acid construct is DNA, the DNA construct can be cDNA. In some embodiments, the DNA construct can be circular (for example, a plasmid or a loop). In some embodiments, the DNA construct can be a linear DNA sequence or a circularized DNA sequence. In some embodiments, the DNA construct can be prepared by oligonucleotide synthesis, amplification, or other available recombinant DNA methodology including molecular cloning. In some embodiments where at least one of nucleic acid constructs is RNA, the RNA construct is prepared by oligonucleotide synthesis or in vitro transcription (IVT). In some embodiments where an RNA construct is prepared via IVT, a template for the IVT can be a plasmid DNA that has one or more target control sequences (e.g., see a plasmid map illustrated in FIG. 5, in which a synthetic gene AtmX101bCoV contains the target control sequences) in and/or one or more additional control sequences (e.g., see a plasmid map illustrated in FIG. 6, in which a synthetic gene cR-X contains the additional control sequences) as described herein.

In some embodiments, the one or more nucleic acid constructs containing a control template is a plasmid containing at least one target control sequence derived from a SARS-CoV-2 gene and/or at least one sequence derived from control sequence (e.g., bacteriophage MS2 or human RNase P control sequences). In some embodiments, the at least one target control sequence derived from a SARS-CoV-2 gene is derived from one or more of Orf1a, Orf1b, S, E, M, and N genes. In some embodiments, the at least one target control sequence derived from a SARS-CoV-2 gene is derived from one or more of Orf1a, Orf1b and N genes. In some embodiments, the target control sequences from the SARS-CoV-2 genes have sequences that can be amplified by use of primer pairs using primer sequences selected from SEQ ID NO:1 to SEQ ID NO:10 and SEQ ID NO:11 to SEQ ID NO:20, or the complements thereof. In some embodiments, the target control sequences have one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be generated by use of a first primer selected from selected from SEQ ID NO:1 to SEQ ID NO:10 and a second primer selected from selected from SEQ ID NO:11 to SEQ ID NO:20, or the complements thereof. For the purpose of exemplary illustration, without limiting the scope in any manner, provided herein are maps of two plasmids. The plasmid illustrated in FIG. 5 contains a synthetic gene AtmX101bCoV which can have the target control sequences. The plasmid illustrated in FIG. 6 contains a synthetic gene cR-X which can have the additional control sequences.

In general, there is no limitation on the length (or size) of a plasmid used as a nucleic acid construct in a control template and/or assay. In some embodiments, the size of the plasmid can be anywhere between about 1 kb to several thousands of kb. In some embodiments, the size of the plasmid can be anywhere between about 1 kb to 1,000 kb. In some embodiments, the size of the plasmid can be anywhere between about 1 kb to 500 kb. In some embodiments, the size of the plasmid can be anywhere between about 1 kb to 400 kb. In some embodiments, the size of the plasmid can be anywhere between about 1 kb to 300 kb. In some embodiments, the size of the plasmid can be anywhere between about 1 kb to 200 kb. In some embodiments, the size of the plasmid can be anywhere between about 1 kb to 100 kb. In some embodiments, the size of the plasmid can be anywhere between about 1 kb to 50 kb. In some embodiments, the size of the plasmid can be anywhere between about 1 kb to 25 kb. In some embodiments, the size of the plasmid can be anywhere between about 1 kb to 10 kb. In some embodiments, the size of the plasmid can be anywhere between about 1 kb to 5 kb.

In some embodiments, one plasmid can have one or more target control sequences and/or one or more additional control sequences. In some embodiments, one plasmid has one target control sequence and in some other embodiments, one plasmid has more than one target control sequences. In some embodiments, one plasmid has a bacteriophage MS2 control sequence. In some embodiments, one plasmid has a human RNase P control sequence. In some embodiments, one plasmid has bacteriophage MS2 and human RNase P control sequences. In some embodiments, one plasmid has one or more target control sequence and one or more additional control sequence. In some embodiments, the nucleic acid construct can contain one or more plasmids. Thus, in some embodiments, a control template and/or assay can have one plasmid that has both target control sequence(s) and additional control sequence(s). In some other embodiments, a control template and/or assay can have one plasmid that has target control sequence(s) and a separate plasmid that has additional control sequence(s). In some embodiments, a control template and/or assay can have two or more plasmids that have target control sequences and a separate plasmid that has one or more additional control sequences.

Copy number of a single sequence (i.e., a target control sequence or an additional control sequence) that is included in each plasmid sequence can be one to any natural number. The number of different sequences that are included in a plasmid sequence can be one to any natural number. In some embodiments, the number of different sequences in one plasmid can be 1 to 300, 1 to 200, 1 to 100, 1 to 50, 1 to 25, 1 to 10, 1 to 9, 1 to 8, 1 to 7, 1 to 6, 1 to 5, 1 to 4, 1 to 3, 1 to 2, 1, or any intervening number. Thus, in some embodiments, one plasmid sequence can have one target control sequence or one additional control sequence. In some other embodiments, one plasmid sequence can have two or more different target control sequences. In some other embodiments, one plasmid sequence can have two or more different additional control sequences. In some other embodiments, one plasmid sequence can have one or more different target control sequence and one or more different additional control sequence. In some embodiments, there are a first plasmid that has a first set of target control sequences, a second plasmid that has a second set of target control sequences that is different from the first set of target control sequences, and a third plasmid that has one or more additional control sequences.

In some embodiments, one or more nucleic acid construct from a control template and/or assay is a cDNA sequence containing at least one target control sequence derived from a SARS-CoV-2 gene and/or at least one sequence derived from control sequence (e.g., bacteriophage MS2 or human RNase P control sequences). In some embodiments, the at least one target control sequence derived from a SARS-CoV-2 gene is derived from one or more of Orf1a, Orf1b, S, E, M, and N genes. In some embodiments, the at least one target control sequence derived from a SARS-CoV-2 gene is derived from one or more of Orf1a, Orf1b and N genes. In some embodiments, the target control sequences from the SARS-CoV-2 genes have sequences that can be amplified by use of primer pairs using primer sequences selected from SEQ ID NO:1 to SEQ ID NO:10 and SEQ ID NO:11 to SEQ ID NO:20, or the complements thereof. In some embodiments, the target control sequences have one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:10 and a second primer selected from selected from SEQ ID NO:11 to SEQ ID NO:20, or the complements thereof.

In general, there is no limitation on the length (or size) of a cDNA sequence used as a nucleic acid construct in a control template and/or assay. In some embodiments, the size of the cDNA sequence can be anywhere between about 1 kb to several kbs. In some embodiments, the size of the cDNA sequence can be anywhere between about 1 kb to 100 kb. In some embodiments, the size of the cDNA sequence can be anywhere between about 1 kb to 50 kb. In some embodiments, the size of the cDNA sequence can be anywhere between about 1 kb to 25 kb. In some embodiments, the size of the cDNA sequence can be anywhere between about 1 kb to 10 kb. In some embodiments, the size of the cDNA sequence can be anywhere between about 1 kb to 5 kb. In some embodiments, the size of the cDNA sequence can be anywhere between about 1 kb to 4 kb. In some embodiments, the size of the cDNA sequence can be anywhere between about 1 kb to 3 kb. In some embodiments, the size of the cDNA sequence can be anywhere between about 1 kb to 2 kb. In some embodiments, the size of the cDNA sequence can be anywhere between about 100 bp to 1 kb. In some embodiments, the size of the cDNA sequence can be anywhere between about 100 bp kb 500 bp. In some embodiments, the size of the cDNA sequence can be anywhere between about 200 bp to 1 kb.

In some embodiments, one cDNA sequence can have one or more target control sequences and/or one or more additional control sequences. In some embodiments, one cDNA sequence has one target control sequence and in some other embodiments, one cDNA sequence has more than one target control sequences. In some embodiments, one cDNA sequence has a bacteriophage MS2 control sequence. In some embodiments, one cDNA sequence has a human RNase P control sequence. In some embodiments, one cDNA sequence has bacteriophage MS2 and human RNase P control sequences. In some embodiments, one cDNA sequence has one or more target control sequence and one or more additional control sequence. In some embodiments, the nucleic acid construct can contain one or more cDNA sequences. Thus, in some embodiments, a control template and/or assay can have one cDNA sequence that has both target control sequence(s) and additional control sequence(s). In some other embodiments, a control template and/or assay can have one cDNA sequence that has target control sequence(s) and a separate cDNA sequence that has additional control sequence(s). In some embodiments, a control template and/or assay can have two or more cDNA sequences that have target control sequences and a separate cDNA sequence that has one or more additional control sequences.

Copy number of a single sequence (i.e., a target control sequence or an additional control sequence) that is included in each cDNA sequence can be one to any natural number. The number of different sequences that are included in a cDNA sequence can be one to any natural number. In some embodiments, the number of different sequences in one cDNA can be 1 to 300, 1 to 200, 1 to 100, 1 to 50, 1 to 25, 1 to 10, 1 to 9, 1 to 8, 1 to 7, 1 to 6, 1 to 5, 1 to 4, 1 to 3, 1 to 2, 1, or any intervening number. Thus, in some embodiments, one cDNA sequence can have one target control sequence or one additional control sequence. In some other embodiments, one cDNA sequence can have two or more different target control sequences. In some other embodiments, one cDNA sequence can have two or more different additional control sequences. In some other embodiments, one cDNA sequence can have one or more different target control sequence and one or more different additional control sequence. In some embodiments, there are a first cDNA sequence that has a first set of target control sequences, a second cDNA sequence that has a second set of target control sequences that is different from the first set of target control sequences, and a third cDNA sequence that has one or more additional control sequences.

In some embodiments, one or more nucleic acid construct from a control template and/or assay is an RNA sequence containing at least one target control sequence derived from a SARS-CoV-2 gene and/or at least one sequence derived from control sequence (e.g., bacteriophage MS2 or human RNase P control sequences). RNA sequences can be preferred in some embodiments, especially where target control sequences are derived from RNA viral genome. By using RNA sequences as a control template/assay, which is the same type of target control sequences from a sample (e.g., SARS-CoV-2 sequences from a sample that is collected from a patient), it can create a reaction condition that is substantially similar to the condition that the actual sample nucleic acid is processed.

In some embodiments, the at least one target control sequence derived from a SARS-CoV-2 gene is derived from one or more of Orf1a, Orf1b, S, E, M, and N genes. In some embodiments, the at least one target control sequence derived from a SARS-CoV-2 gene is derived from one or more of Orf1a, Orf1b and N genes. In some embodiments, the target control sequences from the SARS-CoV-2 genes have sequences that can be amplified by use of primer pairs using primer sequences selected from SEQ ID NO:1 to SEQ ID NO:10 and SEQ ID NO:11 to SEQ ID NO:20, or the complements thereof.

In general, there is no limitation on the length (or size) of an RNA sequence used as a nucleic acid construct in a control template and/or assay. In some embodiments, the size of the RNA sequence can be anywhere between about 1 kb to several kbs. In some embodiments, the size of the RNA sequence can be anywhere between about 1 kb to 100 kb. In some embodiments, the size of the RNA sequence can be anywhere between about 1 kb to 50 kb. In some embodiments, the size of the RNA sequence can be anywhere between about 1 kb to 25 kb. In some embodiments, the size of the RNA sequence can be anywhere between about 1 kb to 10 kb. In some embodiments, the size of the RNA sequence can be anywhere between about 1 kb to 5 kb. In some embodiments, the size of the RNA sequence can be anywhere between about 1 kb to 4 kb. In some embodiments, the size of the RNA sequence can be anywhere between about 1 kb to 3 kb. In some embodiments, the size of the RNA sequence can be anywhere between about 1 kb to 2 kb. In some embodiments, the size of the RNA sequence can be anywhere between about 100 bp to 2 kb. In some embodiments, the size of the RNA sequence can be anywhere between about 100 bp to 1.5 kb. In some embodiments, the size of the RNA sequence can be anywhere between about 100 bp to 1 kb. In some embodiments, the size of the RNA sequence can be anywhere between about 100 bp kb 500 bp. In some embodiments, the size of the RNA sequence can be anywhere between about 500 bp to 1.5 kb. In some embodiments, the size of the RNA sequence can be anywhere between about 500 bp to 2 kb.

In some embodiments, one RNA sequence can have one or more target control sequences and/or one or more additional control sequences. In some embodiments, one RNA sequence has one target control sequence and in some other embodiments, one RNA sequence has more than one target control sequences. In some embodiments, one RNA sequence has a bacteriophage MS2 control sequence. In some embodiments, one RNA sequence has a human RNase P control sequence. In some embodiments, one RNA sequence has bacteriophage MS2 and human RNase P control sequences. In some embodiments, one RNA sequence has one or more target control sequence and one or more additional control sequence. In some embodiments, the nucleic acid construct can contain one or more RNA sequences. Thus, in some embodiments, a control template and/or assay can have one RNA sequence that has both target control sequence(s) and additional control sequence(s). In some other embodiments, a control template and/or assay can have one RNA sequence that has target control sequence(s) and a separate RNA sequence that has additional control sequence(s). In some embodiments, a control template and/or assay can have two or more RNA sequences that have target control sequences and a separate RNA sequence that has one or more additional control sequences.

Copy number of a single sequence (i.e., a target control sequence or an additional control sequence) that is included in each RNA sequence can be one to any natural number. The number of different sequences that are included in an RNA sequence can be one to any natural number. In some embodiments, the number of different sequences in one RNA can be 1 to 300, 1 to 200, 1 to 100, 1 to 50, 1 to 25, 1 to 10, 1 to 9, 1 to 8, 1 to 7, 1 to 6, 1 to 5, 1 to 4, 1 to 3, 1 to 2, 1, or any intervening number. Thus, in some embodiments, one RNA sequence can have one target control sequence or one additional control sequence. In some other embodiments, one RNA sequence can have two or more different target control sequences. In some other embodiments, one RNA sequence can have two or more different additional control sequences. In some other embodiments, one RNA sequence can have one or more different target control sequence and one or more different additional control sequence. In some embodiments, there are a first RNA sequence that has a first set of target control sequences, a second RNA sequence that has a second set of target control sequences that is different from the first set of target control sequences, and a third RNA sequence that has one or more additional control sequences.

In some embodiments, the nucleic acid construct used in a control template and/or assay has any combination of plasmid, cDNA and RNA sequences. Thus, in some embodiments, the nucleic acid construct has one RNA sequence that has one or more target control sequence and one cDNA sequence that has one or more additional control sequence. In some other embodiments, the nucleic acid construct has one RNA sequence that has one or more target control sequence and one plasmid sequence that has one or more additional control sequence. In some alternative embodiments, the target control sequences are provided in a cDNA sequence or plasmid and the additional control sequence(s) can be provided in an RNA sequence.

By way of an exemplary embodiment, without limiting the scope in any manner, provided herein is the sequences of SEQ ID NO:41, SEQ ID NO:42, SEQ ID NO:45, and SEQ ID NO:46. These sequences present DNA sequences that have one or more sequences that can be amplified with primer sequences selected from SEQ ID NO:1 to SEQ ID NO:10 and SEQ ID NO:11 to SEQ ID NO:20 or complements thereof. In some embodiments, two or more sequences among SEQ ID NO:41, SEQ ID NO:42, SEQ ID NO:45, and SEQ ID NO:46 can be provided in a single plasmid or cDNA sequence that can be used as a nucleic acid construct in a control template and/or assay. In some other embodiments, two or more sequences among SEQ ID NO:41, SEQ ID NO:42, SEQ ID NO:45, and SEQ ID NO:46 can be provided in two or more separate cDNA sequences or plasmids. In some other embodiments, one of the sequences (e.g., SEQ ID NO:41) can be provided in a plasmid and another sequence (e.g., SEQ ID NO:42) can be provided in cDNA sequence. In some other embodiments, at least one of the sequences can be provided in DNA (e.g., in a plasmid or cDNA) and at least another sequence can be provided in RNA (SEQ ID NO:43, SEQ ID NO:44, SEQ ID NO:47, and SEQ ID NO:48 are RNA sequences corresponding to SEQ ID NO:41, SEQ ID NO:42, SEQ ID NO:45, and SEQ ID NO:46, respectively).

In some embodiments, a nucleic acid construct used in a control template and/or assay contains one or more sequences selected from the group consisting of SEQ ID NO:41 to SEQ ID NO:48. In some embodiments, the nucleic construct contains one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to the sequences selected from the group consisting of SEQ ID NO:41 to SEQ ID NO:48.

In some embodiments, a target control sequence included in the nucleic acid construct from a control template and/or assay has one or more extra sequence in addition to a sequence that can be amplified by use of primer pairs using primer sequences selected from SEQ ID NO:1 to SEQ ID NO:10 and SEQ ID NO:11 to SEQ ID NO:20 or the complements thereof. In some embodiments, the extra sequence can be added to one or both of 5′ end and 3′ ends of the amplifying sequence in the nucleic acid construct. Therefore, in one example where a target control sequence has a sequence that can be amplified by the first primer sequence of SEQ ID NO:1 and the second primer sequence of SEQ ID NO:11, the nucleic acid construct can have an extra sequence at the 5′ end or 3′ end of the amplifying sequence, or alternatively two extra sequences at both ends of the amplifying sequences. In some embodiments, the extra sequence is a SARS-CoV-2 sequence that is adjacent to the amplifying sequence from the viral genome. In some embodiments, the extra sequence is a random or synthetic sequence. The extra sequence can be in any length, for example, from one nucleotide to several hundreds to thousands of nucleotides. In some embodiments, the extra sequence can be anywhere between one nucleotide to one thousand nucleotides. In some embodiments, the extra sequence can be anywhere between one nucleotide to one hundred nucleotides. In some embodiments, the extra sequence can be anywhere between one nucleotide to fifty nucleotides. In some embodiments, the extra sequence can be anywhere between one nucleotide to twenty-five nucleotides.

In some embodiments where the nucleic acid construct from a control template and/or assay has a plasmid, the plasmid can be derived from a first plasmid that can be replicated in cells such as E. coli. In some other embodiments, the first plasmid can be replaced in other cell systems such as yeast, animal cells or mammalian cells. In some embodiments, one or more target control sequences and/or one or more additional control sequences are prepared via oligonucleotide synthesis or amplification (e.g., PCR) and assembled into the first plasmid. The assembled plasmid can be transformed into bacteria for amplification. The amplified plasmids can be purified, and the concentration of the purified plasmids can be determined, e.g., by UV spectroscopy. The final construct can be verified by sequencing.

In some embodiments where the nucleic acid construct from a control template and/or assay has a cDNA sequence, the cDNA sequence can be prepared via reverse transcription using its respective RNA template. In some embodiments, viral RNA sequences (e.g., SARS-CoV-2 genome sequences or fragment thereof) can be used as RNA template sequences to reverse transcribe corresponding cDNA sequences. In some embodiments where the nucleic acid construct has cDNA sequence having RNase P control sequence, a human RNA sequence of RNase P or fragment thereof can be used as an RNA template to prepare the cDNA. The resulting cDNA, after purification and quantification, can be used as a nucleic acid construct.

In some embodiments where the nucleic acid construct from a control template and/or assay has an RNA sequence, the RNA sequence can be prepared via oligonucleotide synthesis or in vitro transcription (IVT). Various kinds of sequences can be used as a template for the RNA sequence. In some embodiments, synthetic DNA sequence that has a target control sequence can be used as a template for the RNA sequence. In some embodiments, DNA sequences amplified from a viral genomic sequence can be used as a template for the RNA sequence. In some embodiments, a plasmid described herein, which can be used by itself as a nucleic acid construct in a control template and/or assay, can be used as a template for the RNA sequence. In some of such embodiments, a plasmid can be prepared by assembling one or more desired target control sequences and also has necessary sequences for in vitro transcription, e.g., T7 promoter. Thus, the resulting RNA sequences that is generated via IVT using the plasmid as the template will have the target control sequences present in the plasmid.

In general, there is no limitation on a copy number of a nucleic acid construct to be used in an assay. Preferred copy number can differ depending on a sequence. In some embodiments, the copy number of target control sequence used in an assay can be about 1 to 100,000 copies, about 1 to 50,000 copies, about 1 to 10,000 copies, about 1 to 5,000 copies, about 1 to 2,500 copies, about 1 to 1,000 copies, about 1 to 500 copies, about 1 to 250 copies, about 1 to 100 copies, about 1 to 50 copies, about 1 to 45 copies, about 1 to 40 copies, about 1 to 35 copies, about 1 to 30 copies, about 1 to 25 copies, about 1 to 20 copies, about 1 to 15 copies, about 1 to 10 copies, or about 1 to 5 copies. In some embodiments, the copy number of additional control sequence (e.g., MS2 or RNase P control sequence) used in an assay can be about 1 to 100,000 copies, about 1 to 50,000 copies, about 1 to 10,000 copies, about 1 to 5,000 copies, about 1 to 2,500 copies, about 1 to 1,000 copies, about 1 to 500 copies, about 1 to 250 copies, about 1 to 100 copies, about 1 to 50 copies, about 1 to 45 copies, about 1 to 40 copies, about 1 to 35 copies, about 1 to 30 copies, about 1 to 25 copies, about 1 to 20 copies, about 1 to 15 copies, about 1 to 10 copies, or about 1 to 5 copies.

In some embodiments, the copy number of target control sequence and the copy number of additional control sequence (e.g., MS2 or RNase P) used in an assay can be identical, similar or different. In some embodiments, the ratio between the copy numbers of target control sequence and additional control sequence used in an assay can be about 1:1, about 2:1, about 3:1, about 4:1, about 5:1, about 6:1, about 7:1, about 8:1, about 9:1, about 10:1, about 20:1, about 30:1, about 40:1, about 50:1, about 100:1, about 1:1, about 1:2, about 1:3, about 1:4, about 1:5, about 1:6, about 1:7, about 1:8, about 1:9, about 1:10, about 1:20, about 1:30, about 1:40, about 1:50, or about 1:100.

In some embodiments, provided herein are cells or other compositions comprising the one or more nucleic acid construct from a control template and/or assay as described herein. In some embodiments, provided herein are cells or other compositions comprising one or more nucleic acid constructs that is in a form of plasmid.

In one aspect, one or more nucleic acid construct from a control template and/or assay as described herein can be used in compositions and methods for use in monitoring, evaluating, and/or troubleshooting nucleic acid amplification and/or extraction workflows. In some embodiments, the nucleic acid construct as described herein, which can be in a form of plasmid, cDNA, or RNA or any combination thereof, can be used as a positive nucleic acid control molecule or reagent. In some embodiments, the nucleic acid construct, when used as a control nucleic acid molecule or reagent, includes the same or overlapping target control sequences to which an amplification and/or detection assay is directed. In some embodiments, the control nucleic acid molecule includes a subset of the target control sequences to which an amplification and/or detection assays is directed. In some embodiments, the control nucleic acid molecule includes additional target control sequences to which additional reference or control assays are directed.

In another aspect, provided herein are methods, compositions, and kits for amplifying a plurality of nucleic acid target control sequences in a sample. In some embodiments, the target control sequences are derived from SARS-CoV-2. In some embodiments, the method provides distributing the sample into a plurality of reaction volumes wherein the reaction volumes include at least two different pair of amplification primers configured to amplify a corresponding target control sequence. In some embodiments, the method also includes providing a nucleic acid construct serving as a control template/assay. In some embodiments, the nucleic acid construct contains the target control sequences and one or more additional control sequences (e.g., MS2 and/or RNase P control sequences). The nucleic acid construct may undergo the same amplification reaction with the amplification primers. In some embodiments, the amplification reaction with the nucleic acid construct provides amplicons of target control sequences and amplicons of additional control sequences and this presence of amplicons may be indicative of positive amplification reaction.

In another aspect, provided herein are compositions and kits for detecting one or more target nucleic acids. In some embodiments, the compositions and kits detect one or more target control sequences that are derived from SARS-CoV-2 as described herein. In some embodiments, the compositions and kits have a control template and/or assay that has one or more nucleic construct. In some embodiments, the one or more nucleic acid construct has the target control sequences derived from SARS-CoV-2. In some embodiments, the nucleic acid construct has one or more additional control sequence such as bacteriophage MS2 and/or human RNase P sequence. In some embodiments, the nucleic acid construct is in form of DNA or RNA. In some embodiments, the nucleic acid construct is in form of a plasmid, cDNA or RNA. In some embodiments, the compositions and kits contain cells (e.g., E. coli cells) that contain the nucleic acid construct which is in form of plasmid. In some embodiments, the nucleic acid construct contains one or more sequences selected from the group consisting of SEQ ID NO:41 to SEQ ID NO:48. In some embodiments, the nucleic construct contains one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to the sequences selected from the group consisting of SEQ ID NO:41 to SEQ ID NO:48.

In another aspect, provided herein are methods for producing compositions and kits that can detect one or more target nucleic acids. In some embodiments, the target control sequences are derived from SARS-CoV-2 as described herein. The method contains a step of producing a control template and/or assay that has one or more nucleic construct. In some embodiments, the one or more nucleic acid construct has the target control sequences derived from SARS-CoV-2. In some embodiments, the nucleic acid construct has one or more additional control sequence such as bacteriophage MS2 and/or human RNase P sequence. In some embodiments, the nucleic acid construct is in form of DNA or RNA. In some embodiments, the nucleic acid construct is in form of a plasmid, cDNA or RNA. In some embodiments where the nucleic acid construct is RNA, the construct can be prepared via in vitro transcription of a DNA template that encodes the target control sequences and/or the additional control sequences. In some embodiments, the DNA template for in vitro transcription is a plasmid that has one or more sequences derived from SARS-CoV-2. In some embodiments, the DNA template for in vitro transcription is a plasmid that has one or more sequences derived from bacteriophage MS2 and human RNase P genes. In some embodiments, a plasmid serving as a DNA template has one or more sequences that can be generated by a primer pair selected from SEQ ID NO:1 to SEQ ID NO:10 and SEQ ID NO:11 to SEQ ID NO:20 or complement thereof.

In some embodiments, a plasmid serving as a DNA template has one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:10 and a second primer selected from selected from SEQ ID NO:11 to SEQ ID NO:20, or the complements thereof. In some embodiments, an RNA sequence that is generated via in vitro transcription using any one of plasmid templates (e.g., plasmids illustrated in FIG. 5 and FIG. 6) described here contains the sequences from the plasmid template or complement thereof. In some embodiments, the RNA sequence can have one or more sequences that can be amplified by a primer pair selected from SEQ ID NO:1 to SEQ ID NO:10 and SEQ ID NO:11 to SEQ ID NO:20 or complement thereof. In some embodiments, the RNA sequence has one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:10 and a second primer selected from selected from SEQ ID NO:11 to SEQ ID NO:20, or the complements thereof.

In one aspect, provided herein is a composition useful for biological assays. The composition may contain a first target control sequence containing a nucleic acid sequence derived from a coronavirus and a control sequence containing a nucleic acid sequence that is not derived from a coronavirus. In some embodiments, the first target control sequence and the control sequence are located on the same nucleic acid molecule. In some other embodiments, the first target control sequence and the control sequence are located on different nucleic acid molecules. In some embodiments, one or both the first target control sequence and the control sequence are located within a plasmid. In some other embodiments, one or both the first target control sequence and the control sequence are located within a cDNA sequence. In still some other embodiments one or both the first target control sequence and the control sequence are located within an RNA sequence. In some embodiments, the control sequence is derived from the human genome. In some embodiments, the control sequence is derived from the genes for human GADPH or RNase P (RPPH1) or from regions of both. In some embodiments, the first target control sequence is derived from the coronavirus SARS-CoV-2. In some embodiments, the first target control sequence is derived from genes of the coronavirus SARS-CoV-2 selected from the group consisting of: the S gene encoding the Spike protein; the N gene encoding the nucleocapsid protein, the M gene encoding the Membrane protein, the ORF1a gene encoding open reading frame 1a, and the ORF1b gene encoding the open reading frame 1b. In some embodiments, the first target control sequence is derived from genes of the coronavirus SARS-CoV-2 selected from the group consisting of: the N gene encoding the nucleocapsid protein, the M gene encoding the Membrane protein, the ORF1a gene encoding open reading frame 1a, and the ORF1b gene encoding the open reading frame 1b. In some embodiments, the composition further includes a second target control sequence derived from a coronavirus. In some embodiments, the first target control sequence is derived from the N gene, the second target control sequence is derived from the ORF1a gene, and the control sequence is derived from the human RPPH1 gene encoding RNase P. In some embodiments, the first target control sequence is derived from the N gene, the second target control sequence is derived from the ORF1b gene, and the control sequence is derived from the human RPPH1 gene encoding RNase P. In some embodiments, the composition further includes a third target control sequence derived from a coronavirus. In some embodiments, the first target control sequence is derived from the N gene, the second target control sequence is derived from the ORF1a gene, the first target control sequence is derived from the ORF1b gene, and the control sequence is derived from the human RPPH1 gene encoding RNase P. In some embodiments, the composition further includes a fourth target control sequence derived from a coronavirus. In some embodiments, the composition contains one or more sequences that can be amplified by a primer pair selected from the group consisting of SEQ ID NO:1 to SEQ ID NO:10 and SEQ ID NO:11 to SEQ ID NO:20 or complement thereof. In some embodiments, the composition contains one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:10 and a second primer selected from selected from SEQ ID NO:11 to SEQ ID NO:20, or the complements thereof. In some embodiments, the composition contains one or more sequences selected from the group consisting of the sequences of SEQ ID NO:41 to SEQ ID NO:48. In some embodiments, the composition contains one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to the sequences selected from the group consisting of SEQ ID NO:41 to SEQ ID NO:48.

In another aspect, provided herein is a method for detecting coronaviral nucleic acid sequences in a biological sample. The method may have providing an amplification reaction mixture, which may contain a portion of a biological sample including or derived from a living organism, at least one forward primer and at least one reverse primer, nucleotides and a polymerase enzyme, and a composition described herein. The method may have subjecting the amplification reaction mixture to nucleic acid amplification conditions.

In another aspect, provided herein is a method of preparing an RNA sequence. The method may contain providing a plasmid that comprises one or more sequences derived from SARS-CoV-2 and/or complement thereof and subjecting the plasmid to an in vitro transcription reaction wherein an RNA sequence is transcribed using the plasmid as a template. In some embodiments, the one or more sequences derived from SARS-CoV-2 and/or complement thereof is selected from the group consisting of sequences that can be amplified with a primer pair selected from SEQ ID NO:1 to SEQ ID NO:8 and SEQ ID NO:11 to SEQ ID NO:18 or complement thereof. In some embodiments, the one or more sequences derived from SARS-CoV-2 and/or complement thereof comprises one or more sequences that can be amplified by a primer pair selected from the group consisting of SEQ ID NO:1 to SEQ ID NO:8 and SEQ ID NO: 11 to SEQ ID NO:18 or complement thereof. In some embodiments, the one or more sequences derived from SARS-CoV-2 and/or complement thereof comprises one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:8 and a second primer selected from selected from SEQ ID NO:11 to SEQ ID NO:18, or the complement thereof. In some embodiments, the RNA sequence comprises two or more, three or more, four or more, five or more, six or more, seven or more, eight or more, nine or more, or ten or more sequences that are derived from SARS-CoV-2 and/or complements thereof. In some embodiments, the method further contains providing another plasmid that comprises one or more sequences encoding bacteriophage MS2 gene and/or human RPPH1 gene encoding RNase P or complement thereof. In some embodiments, the plasmid further contains one or more additional sequences encoding bacteriophage MS2 gene and/or human RPPH1 gene encoding RNase P or complement thereof.

### EXAMPLES

### Example 1: Multiplex Assay for Detecting SARS-CoV-2

An exemplary protocol for detecting SARS-CoV-2 from a biological sample via a multiplex assay was performed using the TaqPath™ COVID-19 Combo Kit (Thermo Fisher Scientific, Catalog No. A47813) or the TaqPath™ COVID-19 Combo Kit Advanced (Thermo Fisher Scientific, Catalog No. A47814). The kits are similar but with some different reagent volumes for workflows of different sample volumes. The assay kit included a “COVID-19 Real Time PCR Assay Multiplex” component that included primers and FAM-labeled probes for detecting Orf1ab, primers and ABY-labeled probes for detecting S protein, and primers and VIC-labeled probes for detecting N protein coding sequences for SARS-CoV-2, as well as a JUN-labeled internal positive control directed to either endogenous RNase P or an exogenous MS2 RNA template. The assay kit also included a synthetic DNA construct COVID-19 Control (1×104 copies/μL) encoding the target sequences for Orf1ab, S protein, and N protein.

The total nucleic acid content was isolated from samples collected via nasopharyngeal swab, nasopharyngeal aspirate, or bronchoalveolar lavage using the MagMAX Viral/Pathogen Nucleic Acid Isolation Kit (Thermo Fisher Scientific, Catalog No. A42356) in accordance with the instructions provided therewith.

For each assay, the components in Table 7 were combined for the number of reactions, plus 10% overage:

TABLE 7
RT-qPCR Reaction Mix

| Component | Volume / reaction | |
|---|---|---|
| Master Mix (4X) | 6.25 | Ml |
| COVID-19 Real Time PCR | 1.25 | μL |
| Assay Multiplex | | |
| Nuclease-free water | 12.50 | μL |
| Total Reaction Mix Volume | 20.00 | μL |

The “Master Mix” referenced in Table 12 was a TaqPath™ 1-Step Multiplex Master Mix (No ROX™) (Thermo Fisher Scientific, Catalog Nos. A28521, A28522, A28523).

The COVID-19 Control was diluted to a working stock of 25 copies/μL. The reaction mixes were vortexed for about 10-30 seconds) and centrifuged briefly. For each reaction, the components in Table 8, below, were combined in a MicroAmp™ Optical 96-Well Reaction Plate (0.2 mL/well) (Thermo Fisher Scientific, Catalog No. N8010560):

TABLE 8
RT-qPCR Reactions

| Component | Volume / reaction |
|---|---|
| Reaction Mix (see Table 12) | 20.0 μL |
| Nucleic acid sample (from RNA extraction) or | 5.00 μL |
| 2 μL COVID-19 Control + 3 μL PCR-grade | |
| water or | |
| Purified Negative Control (from RNA | |
| extraction) | |
| Total Reaction Volume | 25.00 μL |

The plate was sealed with a MicroAmp Optical Adhesive Film (Thermo Fisher Scientific, Catalog No. 4306311) and vortexed briefly to mix the contents. The plate was centrifuged briefly to collect the contents at the bottom of the wells. The plate was loaded into a QuantStudio 5 Real-Time PCR System (Thermo Fisher Scientific, Catalog No. A28139) and the protocol in Table 9 was run.

TABLE 9
RT-qPCR Protocol for Multiplex Assay

| Step | Stage | # of cycles | Temp. | | Time | |
|---|---|---|---|---|---|---|
| UNG incubation | 1 | 1 | 25° | C. | 2 | min |
| Reverse transcription^{†} | 2 | 1 | 53° | C.^{†} | 10 | min |
| Polymerase activation^{‡} | 3 | 1 | 95° | C.^{‡} | 2 | min |
| Amplification | 4 | 40 | 95° | C. | 3 | sec |
| | | | 60° | C. | 30 | sec |

^{†}Preferably any temperature between 48° C.-55° C.
^{‡}RT inactivation, initial denaturation, and activation of DNAP.

The resulting data were analyzed using the QuantStudio Design and Analysis Software v1.5.1 included with the QuantStudio 5 Real-Time PCR System. For each plate, the control reactions were confirmed to perform as expected (i.e., the no template control had an undetermined Cₜ value and the positive control had a Cₜ value less than or equal to 30).

The Cₜ value for each individual assay was also analyzed in accordance with Table 10.

TABLE 10
Multiplex Assay Results Guide

| | Ct < 37 | 37 ≤ Ct < 40 | Ct = 40 or undetermined |
|---|---|---|---|
| Orf1ab (FAM) | Positive | Repeat test | Negative |
| N protein (VIC) | Positive | Repeat test | Negative |
| S protein (ABY) | Positive | Repeat test | Negative |
| Pos. Control (JUN) | Positive | Positive | Negative |

The results for each tested sample was interpreted to have SARS-CoV-2 RNA present if either (i) any two of Orf1ab, S protein, or N protein were positive or (ii) any one of Orf1ab, S protein, or N protein were positive in two different samples collected from the same subject. SARS-CoV-2 RNA was not present in the sample if all three of Orf1ab, S protein, and N protein were negative.

### Example 2: Preparation of Sample Matrix

Nasopharyngeal (“NP”) samples and nasal swab (“NS”) samples were collected, pooled, and utilized as a sample matrix into which known concentrations of virion copies were spiked to generate samples. NP and NS samples were purchased from multiple different vendors. Sample volumes ranged from 1-3 ml. When obtained from a vendor as a pre-pooled collection of samples, the total volume of any pool did not exceed 20 ml.

Each sample was tested (3 replicates) to confirm that samples were negative for the targets SARS-CoV-2, Flu A, Flu B, RSV A, and RSV B. Samples that tested negative for all targets were pooled to make a series of 200 ml pools. Following pooling, 20 replicates for each of the 200 ml pools were retested to confirm that samples were negative for all targets. TaqPath™ COVID-19, FluA/B RSV Combo Kit Instructions for Use (Thermo Fisher Scientific, MAN0019583) automated 400-μL sample input volume workflow was utilized to extract RNA. MS2 was added as positive control to each well and 5 negative controls were included per each RT-PCR plate. Using an Applied Biosystems™ 7500 Fast Real-Time PCR Instrument, the amplification conditions of Table 11 were applied (with 1.4° C./sec ramp rate).

TABLE 11
Thermal Protocol

| Temperature | Time |
|---|---|
| 25° C. | 2 min - Hold |
| 53° C. | 10 min - Hold |
| 85° C. | 2 min - Hold |

| | | |
|---|---|---|
| 95° C. | 3 sec | 46 cycles |
| 60° C. | 30 sec | |

Results were analyzed to confirm negative results for all targets. Sample wells with clear amplification of MS2 positive control and no signal for any of the targets were designated negative. Sample wells that showed clear amplification of MS2 and clear or questionable amplification of one or more of the targets was considered a positive result. Only samples that were confirmed negative for all targets were used as sample matrix. Samples were also required to produce a MS2 Ct value of less than 28. Verified samples were then labeled and stored at −80° C.

### Example 3: qPCR Amplification of SARS-CoV-2 Using an Example Assay (MS2 as Control)

The materials utilized in this Example are shown in Table 12:

TABLE 12
Materials

| | |
|---|---|
| Part/Component | Part Number* |

Sample Plate Setup:

| | |
|---|---|
| Gamma irradiated SARS-CoV-2 virus | BEI Resources, NR-52287 |
| TaqMan ™ Control Dilution Buffer | A49889 |
| TaqPath COVID-19 RT-PCR kit (includes MS2 | A47817 |
| phage tube) | |
| NP Pool Sample (from Example 1) | |

Nucleic Acid Extraction Equipment:

| | |
|---|---|
| KingFisher ™ Flex Magnetic Particle Processor with | 5400630 |
| 96 Deep-Well Head | |
| KingFisher ™ Flex 96 Deep-Well Heating Block | 24075430 |
| KingFisher ™ 96 Deep-Well Plate | 95040450 |
| KingFisher ™ 96 tip comb for DW magnets | 97002534 |
| MicroAmp ™ Clear Adhesive Film | 4306311 |

Nucleic Acid Extraction Reagents:

| | |
|---|---|
| MagMAX ™ Viral/Pathogen II Nucleic Acid | A48383 |
| Isolation Kit | |
| Fisher BioReagents ™ Ethanol, Absolute, Molecular | BP2818100 |
| Biology Grade | |
| Ambion Nuclease-free Water | AM9937 |

qPCR Equipment:

| | |
|---|---|
| Applied Biosystems ™ QuantStudio ™ 5 Real-Time | A28569 |
| PCR Instrument, 96-well, 0.2-mL block (used with | |
| instrument firmware v1.3.3 and QuantStudio ™ | |
| Design and Analysis Software v2.5) | |
| MicroAmp ™ Optical 96-Well Reaction Plate with | 4306737 |
| Barcode, 0.2 mL | |
| MicroAmp ™ Optical Adhesive Film | 4311971 |
| MicroAmp ™ Adhesive Film Applicator | 4333183 |

qPCR Reagents:

| | |
|---|---|
| TaqPath ™ 1-Step Multiplex Master Mix (No | A28523 |
| ROX ™) | |
| Ambion Nuclease-free Water | AM9937 |
| TaqMan ™ SARS-CoV-2 with MS2 Assay 2.0 | A51327 |

*All parts are from Thermo Fisher Scientific except where noted otherwise.

Sample Plate Setup: Verified NP sample matrix from Example 2 was spiked with Gamma irradiated SARS-CoV-2 virus at different copy number concentrations ranging from 1 copy per well to 10⁶ copies per reaction/well. Dilution of the inactivated SARS-CoV-2 (stock concentration of 1.7×10⁶ copies/uL) virus was accomplished with TaqMan™ Control Dilution Buffer in a 10-fold serial dilution, starting at 10⁶ copies per well (final amount in qPCR well) down to 1 copy per well (final amount in qPCR well). An additional set of samples included a concentration of 5 copies/well.

Sample Extraction: Sample extraction was carried out by running script (MVP_2Wash_200_Flex) according to the workflow of Table 13:

TABLE 13
Sample Extraction Workflow
MVP_2Wash_200_Flex

| | Plate | | | |
|---|---|---|---|---|
| Plate ID | position | Plate type | Reagent | Volume per well |
| Wash 1 Plate | 2 | KingFisher | Wash Buffer | 500 μL |
| Wash 2 Plate | 3 | Deepwell 96 | 80% Ethanol | 1000 μL |
| Elution Plate | 4 | Plate | Elution | 50 μL |
| | | | Solution | |

| | | |
|---|---|---|
| Tip Comb | 5 | Place tip comb in Deepwell or standard plate |

Sample Plate is plate position 1. The sample plate will contain the NP pool clinical matrix, spiked-in virus sample at the appropriate dilutions, bead bind mix (as shown in Table 10), Proteinase K, and MS2.

The binding bead mix solution was prepared according to Table 14:

TABLE 14
Binding Bead Mix Solution

| | 1X (275 ul) | Volume for 55 wells |
|---|---|---|
| Binding Solution | 265 | 14,575 uL |
| Magnetic beads (MVPII) | 10 | 550 uL |

Invert Binding Solution 10x gently (avoid creation of excessive foaming); Includes 10% overage

The order of addition of the reagents for sample extraction was as follows: 5 μL proteinase K; 275 μL Binding Bead Mix; 190 μL of each NP sample; 10 μL of inactivated SARS-CoV-2; and 5 μL MS2 phage.

qPCR Amplification: Amplification and analysis were performed using a QuantStudio 5 RealTime PCR System. The Reaction Mix was formulated according to Table 15, with volumes including 20% overage for pipette error.

TABLE 15
qPCR Plate Setup

| | Volume Per Sample or |
|---|---|
| Component | Control |
| TaqPath ™ 1-Step Multiplex Master | 6.25 μL |
| Mix (No ROX) | |
| TaqMan ™ SARS-CoV-2 with MS2 | 1.25 μL |
| Assay 2.0 (20X) | |
| Total Volume | 25 μL |

The “TaqMan™ SARS-CoV-2 with MS2 Assay 2.0” of the Reaction Mix corresponds to the set of assays for Target Nos. 1-8 as shown in FIG. 3, with bacteriophage MS2 (Target No. 10) as the positive control. 7.50 μl of the reaction mix (TaqPath™ 1-Step Multiplex Master Mix (No ROX) and TaqMan™ SARS-CoV-2 with MS2 Assay 2.0) was pipetted into each well of the reaction plate and then combined with 17.5 μL sample (or control) according to Table 16.

TABLE 16
Reaction Plate

| | Volume Per Reaction | | |
|---|---|---|---|
| | | Positive | Negative |
| | Sample | Control | Control |
| Component | Reaction | Reaction | Reaction |
| Reaction Mix | 7.50 μl | 7.50 μl | 7.50 μl |
| NP Sample | 17.5 μl | — | — |
| COVID-19 Plus Control Working | — | 17.5 μl | — |
| Dilution | | | |
| Purified Negative Control | — | — | 17.5 μl |
| Nuclease-free Water | — | — | — |
| Total Volume | 25.0 μl | 25.0 μl | 25.0 μl |

The reaction plate was then sealed with an optical adhesive film, ensuring the film was well sealed around the edges. The plate was vortexed at the highest setting speed for 10-30 seconds with medium pressure while moving the plate around to ensure equal contact on the vortex mixer platform. The reaction plate was then centrifuged for 1-2 minutes at 650 g or greater to remove bubbles and collect the liquid at the bottom of the reaction plate. Samples were run in triplicate, except for the 10⁶ copy samples, which were run in duplicate. A further 8 wells each were included at 10 copies, 5 copies, and 1 copy per well. Two wells were reserved for a negative extraction control (water).

The reporter dye and corresponding targets were as shown in Table 17:

TABLE 17
Reporter Dyes & Targets

| Reporter Dye | Target |
|---|---|
| FAM | Orf1a gene |
| VIC | N gene |
| ABY | Orf1b gene |
| JUN | MS2 control |

The reaction was run using the thermal protocol of Table 18:

TABLE 18
Thermal Protocol

| Step | Temperature | Time | | Number of Cycles |
|---|---|---|---|---|
| UNG incubation | 25° C. | 2 | minutes | 1 |
| Reverse transcription | 53° C. | 10 | minutes | 1 |
| Preincubation | 85° C. | 10 | minutes | 1 |
| Activation | 95° C. | 2 | minutes | 1 |
| Denaturation | 95° C. | 3 | seconds | 40 |
| Anneal/Extension | 60° C. | 30 | seconds | |

FIG. 4A illustrates a series of amplification plots resulting from the above process, showing amplification at viral particle concentrations of 1 copy/well, 5 copies/well, and 10 copies/well. As shown, the protocol generated effective amplification even at concentrations as low as 5 copies/well. Orf1a gene targets were amplified/detected using assays for Target Nos. 1, 2, and 7 (Oa1, Oa2, and Oa7); Orf1b gene targets were amplified/detected using assays for Target Nos. 5 and 6 (Ob5 and Ob6); N gene targets were amplified/detected using assays for Target Nos. 3, 4, and 8 (N3, N4, and N8) (see FIG. 3 for list of assays according to Target No.).

### Example 4: qPCR Amplification of SARS-CoV-2 Using an Example Assay (RNase P as Control)

A protocol similar to that of Example 3 was carried out to determine ability to successfully amplify SARS-CoV-2 nucleic acid within a sample. The protocol was similar to that of Example 3 except that the assay used a human RNase P as positive control rather than bacteriophage MS2. In particular, the “TaqMan™ SARS-CoV-2 with MS2 Assay 2.0” (Thermo Fisher Scientific, part no. A51327) of Example 3 (see Table 14) was replaced by a “TaqMan™ SARS-CoV-2 with RNase P Assay 2.0” (Thermo Fisher Scientific, part no. A51121). The “TaqMan™ SARS-CoV-2 with RNase P Assay 2.0” corresponds to the set of assays for Target Nos. 1-8 as shown in FIG. 3, with human RNase P (Target No. 9) as the positive control.

The order of addition of the reagents for sample extraction was as follows: 5 μL proteinase K; 275 μL Binding Bead Mix; 190 μL of each NP sample; and 10 μL of inactivated SARS-CoV-2. The remainder of the protocol was carried out in the same manner as Example 3.

FIG. 4B illustrates a series of amplification plots resulting from the above process, showing amplification at viral particle concentrations of 1 copy/well, 5 copies/well, and 10 copies/well. As shown, the protocol generated effective amplification even at concentrations as low as 5 copies/well. Orf1a gene targets were amplified/detected using assays for Target Nos. 1, 2, and 7 (Oa1, Oa2, and Oa7); Orf1b gene targets were amplified/detected using assays for Target Nos. 5 and 6 (Ob5 and Ob6); N gene targets were amplified/detected using assays for Target Nos. 3, 4, and 8 (N3, N4, and N8) (see FIG. 3 for list of assays according to Target No.).

### Example Embodiments

Provided below is a non-exhaustive list of numbered items reciting certain preferred embodiments:
1. A method for detecting one or more nucleic acid target regions in a sample, comprising:
   (a) forming a reaction mixture comprising:
      (i) at least a portion of the sample;
      (ii) a first forward primer and a first reverse primer, wherein the first forward primer and the first reverse primer are configured to generate a first amplification product of a first target region if said first target region is present in the sample;
      (iii) a second forward primer and a second reverse primer, wherein the second forward primer and the second reverse primer are configured to generate a second amplification product of a second target region if said second target region is present in the sample
      (iv) a third forward primer and a third reverse primer, wherein the third forward primer and the third reverse primer are configured to generate a third amplification product of a third target region if said third target region is present in the sample, and
      (v) a fourth forward primer and a fourth reverse primer, wherein the fourth forward primer and the fourth reverse primer are configured to generate a fourth amplification product of a fourth target region if said fourth target region is present in the sample;
   (b) subjecting the reaction mixture to nucleic acid amplification conditions; and
   (c) forming at least one of the first, second, third, or fourth amplification products.
2. The method of item 1, wherein the reaction mixture further comprises, optionally, one or more of:
   a first probe configured to associate with a first probe binding sequence within the first target region;
   a second probe configured to associate with a second probe binding sequence within the second target region;
   a third probe configured to associate with a third probe binding sequence within the third target region; or
   a fourth probe configured to associate with a fourth probe binding sequence within the fourth target region.
3. The method of item 1 or item 2, wherein the first and the second target regions are present within the same gene.
4. The method of item 3, wherein the first and the second target regions are present within the Orf1a gene, the Orf1b gene, or the N gene.
5. The method of item 3 or item 12, wherein the first and the second target regions are present within a first target gene, and the third and the fourth target regions are present within a second target gene.
6. The method of item 13, wherein the first target gene is one of the Orf1a gene, the Orf1b gene, or the N gene, and wherein the second target gene is a different one of the Orf1a gene, the Orf1b gene, or the N gene.
7. The method of any one of items 2-14, further comprising detecting formation of the first amplification product by detecting a first signal from a first label in a first detection channel, wherein the first signal indicates formation of the first amplification product.
8. The method of item 15, further comprising detecting an amount of the first amplification product.
9. The method of item 15 or item 16, wherein the first label is attached to, or associated with, the first forward primer or the first reverse primer.
10. The method of item 15 or item 16, wherein the first label is attached to, or associated with, the first probe.
11. The method of any one of items 2-18, further including detecting the formation of the second amplification product by detecting a second signal emitted by a second label in the first detection channel, wherein the second signal indicates formation of the second amplification product.
12. The method of item 19, further comprising detecting an amount of the second amplification product.
13. The method of item 19 or item 20, wherein the second label is attached to, or associated with, the second forward primer or the second reverse primer.
14. The method of item 19 or item 20, wherein the second label is attached to, or associated with, the second probe.
15. The method of any one of items 19-22, wherein the first label and the second label are identical.
16. The method of any one of items 19-22, wherein the first label and the second label are different labels that are detectable in the first detection channel.
17. The method of any one of items 2-24, further including detecting the formation of the third amplification product by detecting a third signal emitted by a third label in a second detection channel, wherein the third signal indicates formation of the third amplification product.
18. The method of item 25, further comprising detecting an amount of the third amplification product.
19. The method of item 25 or item 26, wherein the third label is attached to, or associated with, the third forward primer or the third reverse primer.
20. The method of item 25 or item 26, wherein the third label is attached to, or associated with, the third probe.
21. The method of any one of items 2-28, further including detecting the formation of the fourth amplification product by detecting a fourth signal emitted by a fourth label in the second detection channel, wherein the fourth signal indicates formation of the fourth amplification product.
22. The method of item 29, further comprising detecting an amount of the fourth amplification product.
23. The method of item 29 or item 30, wherein the fourth label is attached to, or associated with, the fourth forward primer or the fourth reverse primer.
24. The method of item 29 or item 30, wherein the fourth label is attached to, or associated with, the fourth probe.
25. The method of any one of items 29-32, wherein the third label and the fourth label are identical.
26. The method of any one of items 29-32, wherein the third label and the fourth label are different labels that are detectable in the second detection channel.
27. The method of any one of the preceding items, wherein one or more of the first, second, third, or fourth forward primers are selected from SEQ ID NO:1 to SEQ ID NO:10.
28. The method of any one of the preceding items, wherein one or more of the first, second, third, or fourth reverse primers are selected from SEQ ID NO:11 to SEQ ID NO:20.
29. The method of any one of the preceding items, wherein one or more of the first, second, third, or fourth probes are selected from SEQ ID NO:21 to SEQ ID NO:30.
30. The method of any one of items 1-6, wherein the reaction mixture further includes a fifth forward primer and a fifth reverse primer configured to generate a fifth amplification product of a fifth target region if said fifth target region is present in the sample.
31. The method of item 35, wherein the reaction mixture further includes, optionally, a fifth probe configured to associate with a fifth probe binding sequence within the fifth target region.
32. The method of item 36, further including detecting the formation of the fifth amplification product by detecting a fifth signal emitted by a fifth label in a third detection channel, wherein the fifth signal indicates formation of the fifth amplification product.
33. The method of item 37, further comprising detecting an amount of the fifth amplification product.
34. The method of item 37 or item 38, wherein the fifth label is attached to or associated with the fifth forward primer or the fifth reverse primer.
35. The method of item 37 or item 38, wherein the fifth label is attached to or associated with the fifth probe.
36. The method of any one of items 1-40, wherein the reaction mixture further includes a sixth forward primer and a sixth reverse primer configured to generate a sixth amplification product of a sixth target region if said sixth target region is present in the sample.
37. The method of item 41, wherein the reaction mixture further incudes, optionally, a sixth probe configured to associate with a sixth probe binding sequence within the sixth target region.
38. The method of item 42, further including detecting the formation of the sixth amplification product by detecting a sixth signal emitted by a sixth label in the third detection channel, wherein the sixth signal indicates formation of the sixth amplification product.
39. The method of item 43, further comprising detecting an amount of the sixth amplification product.
40. The method of item 43 or item 44, wherein the sixth label is attached to, or associated with, the sixth forward primer or the sixth reverse primer.
41. The method of item 43 or item 44, wherein the sixth label is attached to, or associated with, the sixth probe.
42. The method of any one of items 43-46, wherein the fifth label and the sixth label are identical.
43. The method of any one of items 43-46, wherein the fifth label and the sixth label are different labels that are detectable in the third detection channel.
44. The method of any one of items 41-48, wherein the fifth and the sixth target regions are present within a third target gene.
45. The method of item 49, wherein the third target gene is one of the Orf1a gene, the Orf1b gene, or the N gene.
46. The method of item 50, wherein the first target gene is a first one of the Orf1a gene, the Orf1b gene, or the N gene, the second target gene is a different, second one of the Orf1a gene, the Orf1b gene, or the N gene, and the third target gene is a different, third one of the Orf1a gene, the Orf1b gene, or the N gene.
47. The method of any one of items 42-51, wherein one or both of the fifth or sixth forward primers are selected from SEQ ID NO:1 to SEQ ID NO:10.
48. The method of any one of items 42-52, wherein one or both of the fifth or sixth reverse primers are selected from SEQ ID NO:11 to SEQ ID NO:20.
49. The method of any one of items 42-53, wherein one or both of the fifth or sixth probes are selected from SEQ ID NO:21 to SEQ ID NO:30.
50. The method of any one of items 1-54, wherein the reaction mixture further includes a seventh forward primer and a seventh reverse primer configured to generate a seventh amplification product of a seventh target region if said seventh target region is present in the sample.
51. The method of item 55, wherein the reaction mixture further incudes, optionally, a seventh probe configured to associate with a seventh probe binding sequence within the seventh target region.
52. The method of item 56, further including detecting the formation of the seventh amplification product by detecting a seventh signal emitted by a seventh label in a fourth detection channel, wherein the seventh signal indicates formation of the seventh amplification product.
53. The method of item 57, further comprising detecting an amount of the seventh amplification product.
54. The method of item 57 or item 58, wherein the seventh label is attached to, or associated with, the seventh forward primer or the seventh reverse primer.
55. The method of item 57 or item 58, wherein the seventh label is attached to, or associated with, the seventh probe.
56. The method of any one of items 55-60, wherein the seventh target region is within the first target gene.
57. The method of any one of items 1-61, wherein the reaction mixture further includes an eighth forward primer and an eighth reverse primer configured to generate an eighth amplification product of an eighth target region if said eighth target region is present in the sample.
58. The method of item 62, wherein the reaction mixture further incudes, optionally, an eighth probe configured to associate with an eighth probe binding sequence within the eighth target region.
59. The method of item 63, further including detecting the formation of the eighth amplification product by detecting an eighth signal emitted by an eighth label in the fourth detection channel, wherein the eighth signal indicates formation of the eighth amplification product.
60. The method of item 64, further comprising detecting an amount of the eighth amplification product.
61. The method of item 64 or item 65, wherein the eighth label is attached to, or associated with, the eighth forward primer or the eighth reverse primer.
62. The method of item 64 or item 65, wherein the eighth label is attached to, or associated with, the eighth probe.
63. The method of any one of items 64-67, wherein the seventh label and/or the eighth label are identical.
64. The method of any one of items 64-67, wherein the seventh label and/or the eighth label are different labels that are detectable in the fourth detection channel.
65. The method of any one of items 64-67, wherein the seventh label and/or the eighth label are different labels that are detectable in one of the first, the second, the third or the fourth detection channels.
66. The method of any one of items 63-70, wherein the eighth target region is within the second target gene.
67. The method of any one of items 63-70, wherein the seventh and/or the eighth target regions are present within the first target gene, the second target gene, the third target gene, and/or a fourth target gene.
68. The method of item 72, wherein the seventh target region is within the first target gene and/or the eighth target region is within the second target gene.
69. The method of any one of items 63-73, wherein one or both of the seventh or eighth forward primers are selected from SEQ ID NO:1 to SEQ ID NO:10.
70. The method of any one of items 63-74, wherein one or both of the seventh or eighth reverse primers are selected from SEQ ID NO:11 to SEQ ID NO:20.
71. The method of any one of items 63-75, wherein one or both of the seventh or eighth probes are selected from SEQ ID NO:21 to SEQ ID NO:30.
72. The method of any one of items 1-0, wherein the reaction mixture further includes a ninth forward primer and a ninth reverse primer configured to generate a ninth amplification product of a ninth target region if said ninth target region is present in the sample.
73. The method of item 79, wherein the reaction mixture further incudes, optionally, a ninth probe configured to associate with a ninth probe binding sequence within the ninth target region.
74. The method of item 81, further including detecting the formation of the ninth amplification product by detecting a ninth signal emitted by ninth label in a fifth detection channel, wherein the ninth signal indicates formation of the ninth amplification product.
75. The method of item 82, further comprising detecting an amount of the ninth amplification product.
76. The method of item 82 or 83, wherein the ninth label is attached to, or associated with, the ninth forward primer or the ninth reverse primer.
77. The method of item 82 or 83, wherein the ninth label is attached to, or associated with, the ninth probe.
78. The method of any one of items 81-85, wherein the ninth target region is associated with a positive control sequence.
79. The method of item 86, wherein the positive control sequence is an RNase P sequence or an MS2 sequence.
80. The method of any one of items 1-87, wherein the reaction mixture further includes a tenth forward primer and a tenth reverse primer configured to generate a tenth amplification product of a tenth target region if said tenth target region is present in the sample.
81. The method of item 88, wherein the reaction mixture further incudes, optionally, a tenth probe configured to associate with a tenth probe binding sequence within the tenth target region.
82. The method of item 89, further including detecting the formation of the tenth amplification product by detecting a tenth signal emitted by a tenth label in the fifth detection channel, wherein the tenth signal indicates formation of the tenth amplification product.
83. The method of item 90, further comprising detecting an amount of the tenth amplification product.
84. The method of item 90 or item 91, wherein the tenth label is attached to or associated with the tenth forward primer or the tenth reverse primer.
85. The method of item 90 or item 91, wherein the tenth label is attached to or associated with the tenth probe.
86. The method of any one of items 90-93, wherein the ninth label and/or the tenth label are identical.
87. The method of any one of items 90-93, wherein the ninth label and/or the tenth label are different labels that are detectable in the fifth detection channel.
88. The method of any one of items 88-95, wherein the tenth target region is associated with a positive control sequence.
89. The method of item 96, wherein the positive control sequence is an RNase P sequence or an MS2 sequence.
90. The method of any one of items 88-95, wherein the ninth and/or the tenth target regions are present within a fifth target gene.
91. The method of any one of items 88-98, wherein at least one of the first, second, third, fourth, fifth, sixth, seventh, and/or eighth target regions are within a first target organism, and the ninth and/or tenth target regions are present within a second target organism.
92. The method of any one of items 89-99, wherein one or both of the ninth or tenth forward primers are selected from SEQ ID NO:1 to SEQ ID NO:10.
93. The method of any one of items 89-100, wherein one or both of the ninth or tenth reverse primers are selected from SEQ ID NO:11 to SEQ ID NO:20.
94. The method of any one of items 89-102, wherein one or both of the ninth or tenth probes are selected from SEQ ID NO:21 to SEQ ID NO:30.
95. The method of any one of the preceding items, wherein at least two of the target regions are present on the same gene.
96. The method of any one of the preceding items, wherein at least two of the target regions do not overlap with each other.
97. The method of item 107, wherein at least three, four, or all of the target regions do not overlap with each other.
98. The method of any one of the preceding items, wherein at least one of the target regions is within a first target organism, and at least one of the target regions is within a second target organism.
99. The method of item 109, wherein the wherein the first and the second target regions are within a first target organism, the third and the fourth target regions are present within a second target organism, the fifth and the sixth target regions are present within a third target organism, the seventh and the eighth target regions are present within a fourth target organism, and the ninth and the tenth target regions are present within a fifth target organism.
100. The method of any one of the preceding items, wherein the method enables detection of a target organism despite the presence of one or more mutations in the target organism.
101. The method of any one of the preceding items, wherein at least one of the target regions is associated with SARS-CoV-2.
102. The method of item 112, wherein at least one of the target regions is not in the S gene of SARS-CoV-2.
103. The method of item 112 or item 113, wherein at least one of the target regions is in the N gene of SARS-CoV-2.
104. The method of any one of items 112-114, wherein at least one of the target regions is in the Orf1a gene of SARS-CoV-2.
105. The method of any one of items 112-115, wherein at least one of the target regions is in the Orf1b gene of SARS-CoV-2.
106. The method of any of the preceding items, wherein at least one of the target regions is within a control gene or control nucleic acid sequence.
107. The method of any one of the preceding items, wherein the sample is a crude biological sample.
108. The method of item 118, wherein forming the reaction mixture comprises contacting the crude biological sample with a composition formulated to enable amplification of the one or more target regions, if present in the sample, without isolation of nucleic acid from other components of the crude biological sample.
109. The method of any of the preceding items, wherein at least one of the target regions contains a SNP locus or a mutation site that is within a probe binding site.
110. The method of item 120, further including determining the presence or absence of the SNP or mutation and wherein the reaction mixture includes a probe or primer that can discriminate the SNP or mutation from a reference sequence.
111. The method of any one of the preceding items, wherein the first target region is SEQ ID NO:31.
112. The method of any one of the preceding items, wherein the second target region is SEQ ID NO:32.
113. The method of any one of the preceding items, wherein the third target region is SEQ ID NO:33.
114. The method of any one of the preceding items, wherein the fourth target region is SEQ ID NO:34.
115. The method of any one of items 35-125, wherein the fifth target region is SEQ ID NO:35.
116. The method of any one of items 41-126, wherein the sixth target region is SEQ ID NO:36.
117. The method of any one of items 55-127, wherein the seventh target region is SEQ ID NO:37.
118. The method of any one of items 62-128, wherein the eighth target region is SEQ ID NO:38.
119. The method of any one of the preceding items, wherein the sample comprises one or more of a saliva sample, buccal sample, nasal sample, nasal pharyngeal sample, blood sample, urine sample, or semen sample.
120. The method of any one of the preceding items, wherein the sample is sourced from a human.
121. The method of any one of the preceding items, wherein the sample is sourced from a non-human.
122. The method of any one of the preceding items, further comprising pooling multiple samples prior to performing the method.
123. A composition for amplifying one or more nucleic acid target regions in a sample, comprising:
   (i) a first forward primer and a first reverse primer, wherein the first forward primer and the first reverse primer are configured to generate a first amplification product of a first target region if said first target region is present in the sample;
   (ii) a second forward primer and a second reverse primer, wherein the second forward primer and the second reverse primer are configured to generate a second amplification product of a second target region if said second target region is present in the sample
   (iii) a third forward primer and a third reverse primer, wherein the third forward primer and the third reverse primer are configured to generate a third amplification product of a third target region if said third target region is present in the sample, and
   (iv) a fourth forward primer and a fourth reverse primer, wherein the fourth forward primer and the fourth reverse primer are configured to generate a fourth amplification product of a fourth target region if said fourth target region is present in the sample.
124. The composition of item 134, optionally further comprising one or more of:
   a first probe configured to associate with a first probe binding sequence within the first target region;
   a second probe configured to associate with a second probe binding sequence within the second target region;
   a third probe configured to associate with a third probe binding sequence within the third target region; or
   a fourth probe configured to associate with a fourth probe binding sequence within the fourth target region.
125. The composition of item 134 or item 135, wherein the first and the second target regions are present within the same gene.
126. The composition of item 136, wherein the first and the second target regions are present within the Orf1a gene, the Orf1b gene, or the N gene.
127. The composition of item 136 or item 145, wherein the first and the second target regions are present within a first target gene, and the third and the fourth target regions are present within a second target gene.
128. The composition of item 146, wherein the first target gene is one of the Orf1a gene, the Orf1b gene, or the N gene, and wherein the second target gene is a different one of the Orf1a gene, the Orf1b gene, or the N gene.
129. The composition of any one of items 135-147, wherein one or more of the first, second, third, or fourth forward primers are selected from SEQ ID NO:1 to SEQ ID NO:10.
130. The composition of any one of items 135-136, wherein one or more of the first, second, third, or fourth reverse primers are selected from SEQ ID NO:11 to SEQ ID NO:20.
131. The composition of any one of items 135-138, wherein one or more of the first, second, third, or fourth probes are selected from SEQ ID NO:21 to SEQ ID NO:30.
132. The composition of any one of items 134-140, further comprising:
   a fifth forward primer and a fifth reverse primer, wherein the fifth forward primer and the fifth reverse primer are configured to generate a fifth amplification product of a fifth target region if said fifth target region is present in the sample; and
   a sixth forward primer and a sixth reverse primer, wherein the sixth forward primer and the sixth reverse primer are configured to generate a sixth amplification product of a sixth target region if said sixth target region is present in the sample.
133. The composition of item 148, optionally further comprising one or more of:
   a fifth probe configured to associate with a fifth binding sequence within the fifth target region; or
   a sixth probe configured to associate with a sixth binding sequence within the sixth target region.
134. The composition of item 148 or item 149, wherein the fifth and the sixth target regions are present within a third target gene.
135. The composition of item 150, wherein the third target gene is one of the Orf1a gene, the Orf1b gene, or the N gene.
136. The method of item 151, wherein the first target gene is a first one of the Orf1a gene, the Orf1b gene, or the N gene, the second target gene is a different, second one of the Orf1a gene, the Orf1b gene, or the N gene, and the third target gene is a different, third one of the Orf1a gene, the Orf1b gene, or the N gene.
137. The composition of any one of items 149-152, wherein one or both of the fifth or sixth forward primers are selected from SEQ ID NO:1 to SEQ ID NO:10.
138. The composition of any one of items 149-153, wherein one or both of the fifth or sixth reverse primers are selected from SEQ ID NO:11 to SEQ ID NO:20.
139. The composition of any one of items 149-154, wherein one or both of the fifth or sixth probes are selected from SEQ ID NO:21 to SEQ ID NO:30.
140. The composition of any one of items 134-155, further comprising a seventh forward primer and a seventh reverse primer, wherein the seventh forward primer and the seventh reverse primer are configured to generate a seventh amplification product of a seventh target region if said seventh target region is present in the sample.
141. The composition of item 156, optionally further comprising a seventh probe configured to associate with a seventh binding sequence within the seventh target region.
142. The composition of item 157, wherein the seventh target region is within the first target gene or a fourth target gene.
143. The composition of any one of items 157-158, wherein the seventh forward primer is selected from SEQ ID NO:1 to SEQ ID NO:10.
144. The composition of any one of items 157-159, wherein the seventh reverse primer is selected from selected from SEQ ID NO:11 to SEQ ID NO:20.
145. The composition of any one of items 157-160, wherein the seventh probe is selected from SEQ ID NO:21 to SEQ ID NO:30.
146. The composition of any one of items 134-161, further comprising an eighth forward primer and an eighth reverse primer, wherein the eighth forward primer and the eighth reverse primer are configured to generate an eighth amplification product of an eighth target region if said eighth target region is present in the sample.
147. The composition of item 162, optionally further comprising an eighth probe configured to associate with an eighth binding sequence within the eighth target region.
148. The composition of item 162 or item 163, wherein the eighth target region is within the second target gene.
149. The composition of item any one of items 156-164, wherein the seventh target region is within the first target gene and/or the eighth target region is within the second target gene.
150. The composition of any one of items 163-165, wherein the eighth forward primer is selected from SEQ ID NO:1 to SEQ ID NO:10.
151. The composition of any one of items 163-166, wherein the eighth reverse primer is selected from SEQ ID NO:11 to SEQ ID NO:20.
152. The composition of any one of items 163-167, wherein the eighth probe is selected from SEQ ID NO:21 to SEQ ID NO:30.
153. The composition of any one of items 134-168, further comprising a ninth forward primer and a ninth reverse primer, wherein the ninth forward primer and the ninth reverse primer are configured to generate a ninth amplification product of a ninth target region if said ninth target region is present in the sample.
154. The composition of item 169, optionally further comprising a ninth probe configured to associate with a ninth binding sequence within the ninth target region.
155. The composition of item 169 or item 170, wherein the ninth target region is associated with a positive control sequence.
156. The composition of item 171, wherein the positive control sequence is an RNase P sequence or an MS2 sequence.
157. The composition of any one of items 170-172, wherein the ninth forward primer is selected from SEQ ID NO:1 to SEQ ID NO:10.
158. The composition of any one of items 170-173, wherein the ninth reverse primer is selected from SEQ ID NO:11 to SEQ ID NO:20.
159. The composition of any one of items 170-174, wherein the ninth probe is selected from SEQ ID NO:21 to SEQ ID NO:30.
160. The composition of any one of items 134-175, further comprising a tenth forward primer and a tenth reverse primer, wherein the tenth forward primer and the tenth reverse primer are configured to generate a tenth amplification product of a tenth target region if said tenth target region is present in the sample.
161. The composition of item 176, optionally further comprising a tenth probe configured to associate with a tenth binding sequence within the tenth target region.
162. The composition of item 176 or item 177, wherein the ninth target region is associated with a positive control sequence.
163. The composition of item 178, wherein the positive control sequence is an RNase P sequence or an MS2 sequence.
164. The composition of any one of items 177-179, wherein the tenth forward primer is selected from SEQ ID NO:1 to SEQ ID NO:10.
165. The composition of any one of items 177-180, wherein the tenth reverse primer is selected from SEQ ID NO:11 to SEQ ID NO:20.
166. The composition of any one of items 177-181, wherein the tenth probe is selected from SEQ ID NO:21 to SEQ ID NO:30.
167. The composition of any one of items 134-182, wherein at least two of the target regions are present on the same gene.
168. The composition of any one of items 134-183, wherein at least two of the target regions do not overlap with each other.
169. The composition of item 184, wherein at least three, four, or all of the target regions do not overlap with each other.
170. The composition of any one of items 134-185, wherein at least one of the target regions is within a first target organism, and at least one of the target regions is within a second target organism.
171. The composition of any one of items 134-186, wherein at least one of the target regions is not in the S gene of SARS-CoV-2.
172. The composition of any one of items 134-186, wherein none of the target regions are in the S gene of SARS-CoV-2.
173. The composition of any one of any one of items 134-188, wherein at least one of the target regions is in the N gene of SARS-CoV-2.
174. The composition of any one of items 134-189, wherein at least one of the target regions is in the Orf1a gene of SARS-CoV-2.
175. The composition of any one of items 134-190, wherein at least one of the target regions is in the Orf1b gene of SARS-CoV-2.
176. The composition of any one of items 134-191, wherein at least one of the target regions is within a control gene or control nucleic acid sequence.
177. The composition of any one of the preceding items, further comprising one or more of a test sample, a polymerase, a buffer, and nucleotides.
178. The composition of item 193, wherein the test sample is a crude biological sample.
179. The composition of item 194, wherein the crude biological sample comprises one or more of a saliva sample, buccal sample, nasal sample, nasal pharyngeal sample, blood sample, urine sample, or semen sample.
180. The composition of any one of items 135-195, wherein one or more of the probes contains a detectable label and, optionally, a quencher moiety that quenches the detectable label.
181. The composition of item 196, wherein the detectable label is a fluorescent dye.
182. The composition of item 197, wherein the fluorescent dye is selected from a JUN dye, an ABY dye, a FAM dye, and a VIC dye.
183. The composition of item 197 or item 198, wherein the fluorescent dye of each probe is different from the fluorescent dye of any other probe associated with a different target region.
184. The composition of any one of items 197-199, wherein the fluorescent dye of each probe associated with a particular target region is the same fluorescent dye as any other probe associated with the same target region.
185. The composition of any one of items 146-200, wherein the fluorescent dye of each probe associated with the first target gene is FAM.
186. The composition of any one of items 146-201, wherein the fluorescent dye of each probe associated with the second target gene is VIC.
187. The composition of any one of items 150-202, wherein the fluorescent dye of each probe associated with the third target gene is ABY.
188. The composition of any one of items any one of items 134-203, wherein the first target region is SEQ ID NO:31.
189. The composition of any one of items 134-204, wherein the second target region is SEQ ID NO:32.
190. The composition of any one of items 134-205, wherein the third target region is SEQ ID NO:33.
191. The composition of any one of items 134-206, wherein the fourth target region is SEQ ID NO:34.
192. The composition of any one of items 148-207, wherein the fifth target region is SEQ ID NO:35.
193. The composition of any one of items 148-208, wherein the sixth target region is SEQ ID NO:36.
194. The composition of any one of items 156-209, wherein the seventh target region is SEQ ID NO:37.
195. The composition of any one of items 162-210, wherein the eighth target region is SEQ ID NO:38.
196. A kit for detecting one or more nucleic acid target regions in a sample, comprising:
   a composition as in any one of items 134-211; and
   a treatment buffer formulated for mixing with a crude biological sample to enable analysis of the sample without a nucleic acid extraction or purification step.
197. The kit of item 212, wherein the treatment buffer comprises a surfactant, a protease component, a chelating agent, and a buffering salt.
198. The kit of item 213, wherein the treatment buffer further comprises a disaccharide selected from sucrose, trehalose, or both.
199. The kit of item 213 or item 214, wherein the surfactant comprises: a nonionic detergent selected from one or more of nonyl phenoxypolyethoxylethanol (NP-40), TERGITOL 15-S-9, TRITON X-100, or TWEEN 20; a cationic detergent selected from one or more of benzalkonium chloride (BZK) or didodecyldimethylammonium bromide (DDAB); a zwitterionic detergent selected from one or more of lauryldimethylamine oxide (LDAO), EMPIGEN BB, or ZWITTERGENT 3-14; or combinations thereof.
200. The kit of any one of items 213-215, wherein the protease component comprises proteinase K.
201. The kit of any one of items 213-216, wherein the protease component comprises a mixture of proteases.
202. The kit of item 217, wherein the mixture of proteases comprises a mixture of proteases isolated from a Streptomyces culture.
203. The kit of item 218, wherein the protease component comprises pronase.
204. The kit of any one of items 213-219, wherein the treatment buffer further comprises an antifoam agent comprising silicon and nonionic emulsifiers.
205. A method as in any one of items 1-133, performed using a composition as in any one of items 134-211 or a kit as in any one of items 212-220.
206. The method of item 205, wherein the method provides a limit of detection (LOD) of 10 copies/reaction or less.
207. The method of item 205, wherein the method provides a limit of detection (LOD) of 5 copies/reaction or less.
208. The method of item 205, wherein the method provides a limit of detection (LOD) of 1 copy/reaction.
209. A composition useful for biological assays, comprising:
   a first target control sequence containing a nucleic acid sequence derived from a coronavirus; and
   a control sequence containing a nucleic acid sequence that is not derived from a coronavirus.
210. The composition of item 209, wherein the first target control sequence and the control sequence are located on the same nucleic acid molecule.
211. The composition of item 209, wherein the first target control sequence and the control sequence are located on different nucleic acid molecules.
212. The composition of any one of items 209-211, wherein one or both the first target control sequence and the control sequence are located within a plasmid.
213. The composition of any one of items 209-211, wherein one or both the first target control sequence and the control sequence are located within a cDNA sequence.
214. The composition of any one of items 209-211, wherein one or both the first target control sequence and the control sequence are located within an RNA sequence.
215. The composition of any one of items 209-214, wherein the control sequence is derived from the human genome.
216. The composition of any one of items 209-215, wherein the control sequence is derived from the genes for human GADPH or RNase P (RPPH1) or from regions of both.
217. The composition of any one of items 209-216, wherein the first target control sequence is derived from the coronavirus SARS-CoV-2.
218. The composition of any one of items 209-217, wherein the first target control sequence is derived from genes of the coronavirus SARS-CoV-2 selected from the group consisting of: the S gene encoding the Spike protein; the N gene encoding the nucleocapsid protein, the M gene encoding the Membrane protein, the ORF1a gene encoding open reading frame 1a, and the ORF1b gene encoding the open reading frame 1b.
219. The composition of any one of items 209-218, wherein the first target control sequence is derived from genes of the coronavirus SARS-CoV-2 selected from the group consisting of: the N gene encoding the nucleocapsid protein, the M gene encoding the Membrane protein, the ORF1a gene encoding open reading frame 1a, and the ORF1b gene encoding the open reading frame 1b.
220. The composition of items any one of items 209-219, wherein the composition further includes a second target control sequence derived from a coronavirus.
221. The composition of item 220, wherein the first target control sequence is derived from the N gene, the second target control sequence is derived from the ORF1a gene, and the control sequence is derived from the human RPPH1 gene encoding RNase P.
222. The composition of item 220, wherein the first target control sequence is derived from the N gene, the second target control sequence is derived from the ORF1b gene, and the control sequence is derived from the human RPPH1 gene encoding RNase P.
223. The composition of any one of items 220-222, wherein the composition further includes a third target control sequence derived from a coronavirus.
224. The composition of item 223, wherein the first target control sequence is derived from the N gene, the second target control sequence is derived from the ORF1a gene, the first target control sequence is derived from the ORF1b gene, and the control sequence is derived from the human RPPH1 gene encoding RNase P.
225. The composition of item 224, wherein the composition further includes a fourth target control sequence derived from a coronavirus.
226. The composition of any one of items 209-225, wherein the composition comprises one or more sequences that can be amplified by a primer pair selected from the group consisting of SEQ ID NO:1 to SEQ ID NO:10 and SEQ ID NO:11 to SEQ ID NO:20.
227. The composition of any one of items 209-227, wherein the composition comprises one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:10 and a second primer selected from selected from SEQ ID NO:11 to SEQ ID NO:20, or the complements thereof.
228. The composition of any one of items 209-227, the composition comprises one or more sequences selected from the group consisting of the sequences of SEQ ID NO:41 to SEQ ID NO:48.
229. The composition of any one of items 209-228, the composition comprises one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to the sequences selected from the group consisting of SEQ ID NO:41 to SEQ ID NO:48.
230. A method for detecting coronaviral nucleic acid sequences in a biological sample, comprising:
   providing an amplification reaction mixture containing:
   (a) a portion of a biological sample including or derived from a living organism;
   (b) at least one forward primer and at least one reverse primer;
   (c) nucleotides and a polymerase enzyme;
   (d) a composition of any of items 209-229; and
   (e) subjecting the amplification reaction mixture to nucleic acid amplification conditions.
231. A method of preparing an RNA sequence, the method comprising:
   (a) providing a plasmid that comprises one or more sequences derived from SARS-CoV-2 and/or complement thereof; and
   (b) subjecting the plasmid to an in vitro transcription reaction wherein an RNA sequence is transcribed using the plasmid as a template.
232. The method of item 231, wherein the one or more sequences derived from SARS-CoV-2 and/or complement thereof is selected from the group consisting of sequences that can be amplified with a primer pair selected from SEQ ID NO:1 to SEQ ID NO:8 and SEQ ID NO:11 to SEQ ID NO:18.
233. The composition of any one of items 231-232, wherein the one or more sequences derived from SARS-CoV-2 and/or complement thereof comprises one or more sequences that can be amplified by a primer pair selected from the group consisting of SEQ ID NO:1 to SEQ ID NO:8 and SEQ ID NO:11 to SEQ ID NO:18 or complement thereof.
234. The composition of any one of items 231-233, wherein the one or more sequences derived from SARS-CoV-2 and/or complement thereof comprises one or more sequences that have the sequence similarity of at least or about 99%, at least or about 98%, at least or about 97%, at least or about 96%, at least or about 95%, at least or about 90%, at least or about 85%, at least or about 80%, at least or about 75%, or at least or about 70% to one or more sequences that can be amplified by use of a first primer selected from selected from SEQ ID NO:1 to SEQ ID NO:8 and a second primer selected from selected from SEQ ID NO:11 to SEQ ID NO:18, or complement thereof.
235. The method of any one of items 231-234, wherein the RNA sequence comprises two or more, three or more, four or more, five or more, six or more, seven or more, eight or more, nine or more, or ten or more sequences that are derived from SARS-CoV-2 and/or complements thereof.
236. The method of any one of items 231-235, wherein the method further comprises (c) providing another plasmid that comprises one or more sequences encoding bacteriophage MS2 gene and/or human RPPH1 gene encoding RNase P or complement thereof.
237. The method of any one of items 231-235, wherein the plasmid further comprises one or more additional sequences encoding bacteriophage MS2 gene and/or human RPPH1 gene encoding RNase P or complement thereof.
238. The method, composition or kit of any of items 1-208, further including the methods or compositions of any of items 209-237.

1. A method for detecting one or more nucleic acid target regions in a sample, comprising:
(a) forming a reaction mixture comprising:
(i) at least a portion of the sample;
(ii) a first forward primer and a first reverse primer, wherein the first forward primer and the first reverse primer are configured to generate a first amplification product of a first target region if said first target region is present in the sample;
(iii) a second forward primer and a second reverse primer, wherein the second forward primer and the second reverse primer are configured to generate a second amplification product of a second target region if said second target region is present in the sample
(iv) a third forward primer and a third reverse primer, wherein the third forward primer and the third reverse primer are configured to generate a third amplification product of a third target region if said third target region is present in the sample, and
(v) a fourth forward primer and a fourth reverse primer, wherein the fourth forward primer and the fourth reverse primer are configured to generate a fourth amplification product of a fourth target region if said fourth target region is present in the sample;
(b) subjecting the reaction mixture to nucleic acid amplification conditions; and
(c) forming at least one of the first, second, third, or fourth amplification products.

2. The method of claim 1, wherein the reaction mixture further comprises, optionally, one or more of:
a first probe configured to associate with a first probe binding sequence within the first target region;
a second probe configured to associate with a second probe binding sequence within the second target region;
a third probe configured to associate with a third probe binding sequence within the third target region; or
a fourth probe configured to associate with a fourth probe binding sequence within the fourth target region.

3. The method of claim 2, wherein the first, second, third, or fourth forward primers are selected from SEQ ID NO:1 to SEQ ID NO:10.

4. The method of claim 3, wherein the first, second, third, and fourth forward primers are independently selected from SEQ ID NO:1 to SEQ ID NO:10.

5. The method of claim 2, wherein the first, second, third, or fourth reverse primers are selected from SEQ ID NO:11 to SEQ ID NO:20.

6. The method of claim 5, wherein the first, second, third, and fourth reverse primers are independently selected from SEQ ID NO:11 to SEQ ID NO:20.

7. The method of claim 2, wherein the first, second, third, or fourth probes are selected from SEQ ID NO:21 to SEQ ID NO:30.

8. The method of claim 7, wherein the first, second, third, and fourth probes are independently selected from SEQ ID NO:21 to SEQ ID NO:30.

9. The method of claim 2, wherein:
the first, second, third, or fourth forward primers are selected from SEQ ID NO:1 to SEQ ID NO:10;
the first, second, third, or fourth reverse primers are selected from SEQ ID NO:11 to SEQ ID NO:20; and
the first, second, third, or fourth probes are selected from SEQ ID NO:21 to SEQ ID NO:30.

10. The method of claim 9, wherein:
the first, second, third, and fourth forward primers are independently selected from SEQ ID NO:1 to SEQ ID NO:10;
the first, second, third, and fourth reverse primers are independently selected from SEQ ID NO:11 to SEQ ID NO:20; and
the first, second, third, and fourth probes are independently selected from SEQ ID NO:21 to SEQ ID NO:30.

11. The method of claim 1, wherein the first and the second target regions are present within the same gene.

12. The method of claim 11, wherein the first and the second target regions are present within the Orf1a gene, the Orf1b gene, or the N gene.

13. The method of claim 11, wherein the first and the second target regions are present within a first target gene, and the third and the fourth target regions are present within a second target gene.

14. The method of claim 13, wherein the first target gene is one of the Orf1a gene, the Orf1b gene, or the N gene, and wherein the second target gene is a different one of the Orf1a gene, the Orf1b gene, or the N gene.

15. The method of claim 1, further comprising detecting formation of the first amplification product by detecting a first signal from a first label in a first detection channel, wherein the first signal indicates formation of the first amplification product.

16. The method of claim 15, further comprising detecting an amount of the first amplification product.

17. The method of claim 15, wherein the first label is attached to, or associated with, the first forward primer or the first reverse primer.

18. The method of claim 15, wherein the first label is attached to, or associated with, the first probe.

19. The method of claim 1, further including detecting the formation of the second amplification product by detecting a second signal emitted by a second label in the first detection channel, wherein the second signal indicates formation of the second amplification product.

20. The method of claim 19, further comprising detecting an amount of the second amplification product.

21-254. (canceled)
